(19) United States
(12) Patent Application Publication
(10) Pub. No.: US 2017/0029451 A1
(43) Pub. Date: Feb. 2, 2017

(54) BRANCHED CHAIN ACYCLIC NUCLEOSIDE PHOSPHONATE ESTERS AND METHODS OF SYNTHESIS AND USES THEREOF

(71) Applicant: CHIMERIX, INC., Durham, NC (US)

(72) Inventors: Roy W. WARE, Raleigh, NC (US); Aaron L. DOWNEY, Durham, NC (US); Ernest Randall LANIER, Chapel Hill, NC (US); Phiroze SETHNA, Durham, NC (US); Dean SELLESETH, Durham, NC (US)

(21) Appl. No.: 15/101,647

(22) PCT Filed: Dec. 5, 2014
(86) PCT No.: PCT/US14/68932

§ 371 Date: Jun. 3, 2016

Related U.S. Application Data
(60) Provisional application No. 61/912,407, filed on Dec. 5, 2013.

(51) Int. Cl. C07F 9/6512; A61K 31/675; A61K 45/06

(52) U.S. Cl. CPC C07F 9/65121; A61K 45/06; A61K 31/675

(57) ABSTRACT

The present invention is directed to branched chain nucleoside phosphonate ester compounds and methods of synthesis thereof. The present invention is also directed to pharmaceutical compositions comprising branched chain nucleoside phosphonate ester compounds and methods of treating and/or preventing double stranded DNA viral infection and/or viral infection associated disease or disorder.

### RELATED APPLICATIONS

This application claims priority to, and benefit of, U.S. Provisional Patent Application No. 61/912,407, filed Dec. 5, 2013, the entire content of which is incorporated herein by reference in its entirety.

### TECHNICAL FIELD

This application relates to branched chain acyclic nucleoside phosphonate compounds, analogs, pharmaceutical compositions thereof, and methods of synthesis thereof. The present disclosure also relates to methods for treating viral infections with said branched chain acyclic nucleoside phosphonate compounds, analogs, and pharmaceutical compositions thereof.

### BACKGROUND OF THE INVENTION

Viral infections can have serious adverse effects on individuals and society as a whole. In addition to fatal viral infections such as ebola, even non-fatal infections can have serious economic consequences. For example, in 1999, influenza infection alone in the United States accounted for $1-3 billion in direct medical costs, not to mention $10-15 billion in indirect costs. See Szucs, J. Antimicrob. Chemother. (1999), Topic B, 11-15. Additional viruses such as human cytomegalovirus (HCMV), BK virus (BKV), Epstein-Barr virus (EBV), adenovirus, JC virus (JCV), SV40, MC virus (MCV), KI virus (KIV), WU virus (WUV), vaccinia, herpes simplex virus 1 (HSV-1), herpes simplex virus 2 (HSV-2), human herpes virus 6 (HHV-6), human herpes virus 8 (HHV-8), hepatitis B virus, hepatitis C virus, varicella zoster virus (VZV), variola major, variola minor, smallpox, cowpox, camelpox, monkeypox, poliovirus, ebola virus, Marburg virus, enterovirus, papilloma virus, and human immunodeficiency virus (HIV) can each have significant societal and economic impacts.

Accordingly, development of an effective antiviral treatment effective against viruses such as these is important to improve the health of infected individuals such as transplant patients, and as a public health measure to prevent outbreaks of other pathogenic viruses.

Nucleoside phosphonates (e.g., ribonucleoside derivatives) represent an excellent target class of antivirals to inhibit viruses which rely on viral encoded enzymes using ribonucleotides as substrates, such as certain viral polymerases for many RNA viruses and/or viral helicases for RNA or DNA viruses. However, one block to efficacy for this class of antivirals is the requirement for biochemical modification of the administered agent inside target cells to form the active antiviral nucleoside triphosphate. If a nucleoside is delivered, three phosphorylation steps are required to form the triphosphate. Delivery of nucleoside phosphonates effectively bypasses the first phosphorylation, but exacerbates problems of delivering clinically useful amounts of the charged drug across the lipid bilayers surrounding cells.

Lipid conjugation can be used to disguise oral drugs, including nucleoside phosphonates, as natural compounds that are readily absorbed by the body. Specifically, nucleoside phosphonates can be modified to resemble partially metabolized (monoacyl) phospholipids. In contrast to normal diacylphospholipids, monoacyl lipid-modified nucleosides can readily penetrate the enterocytes lining the lumen of the gut, enter the circulating blood and/or lymph and, unlike standard drugs, remain intact. Consequently, the lipid moiety does more than deliver the nucleoside to the plasma; it facilitates efficient uptake into the target cells. The lipid is cleaved in the cytoplasmic compartment of the target cells and in the case of nucleoside analog conjugates, yields the corresponding monophosphate. Overall, this strategy can lead to greatly increased levels of the active antiviral at the site of viral replication.

This invention addresses the need for new therapies that can be used to treat and/or prevent viral induced disease using novel antivirals and delivery vehicles.

The present disclosure, in part, provides branched chain nucleoside phosphonate esters and methods of synthesis thereof for use as antiviral agents. The present disclosure also provides methods of treating and/or preventing viral infection and/or viral infection associated disease or disorder with one or more compounds of the embodiments.

### SUMMARY OF THE INVENTION

The present invention relates to compounds of Formula (I):

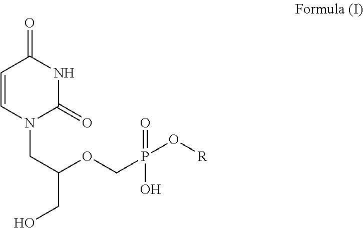

and pharmaceutically acceptable enantiomers, diastereomers, racemates, mixtures or salts thereof, wherein R is:

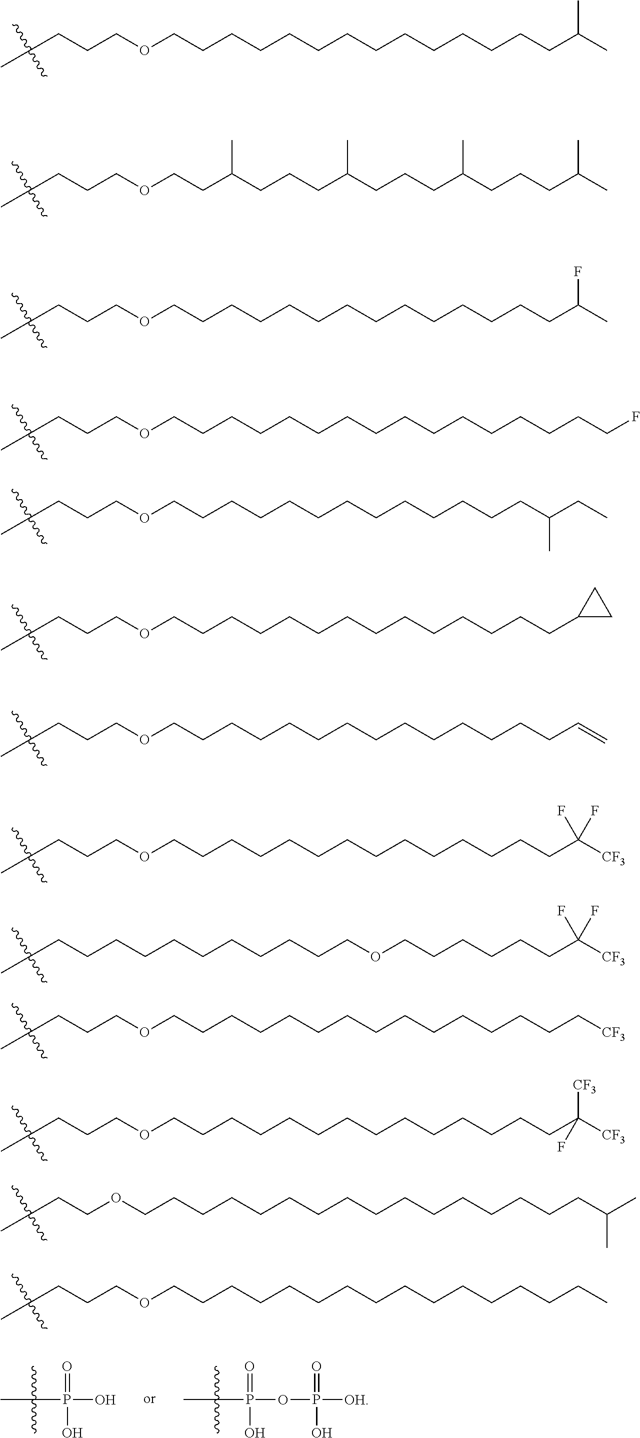

The present invention also relates to the following compounds:

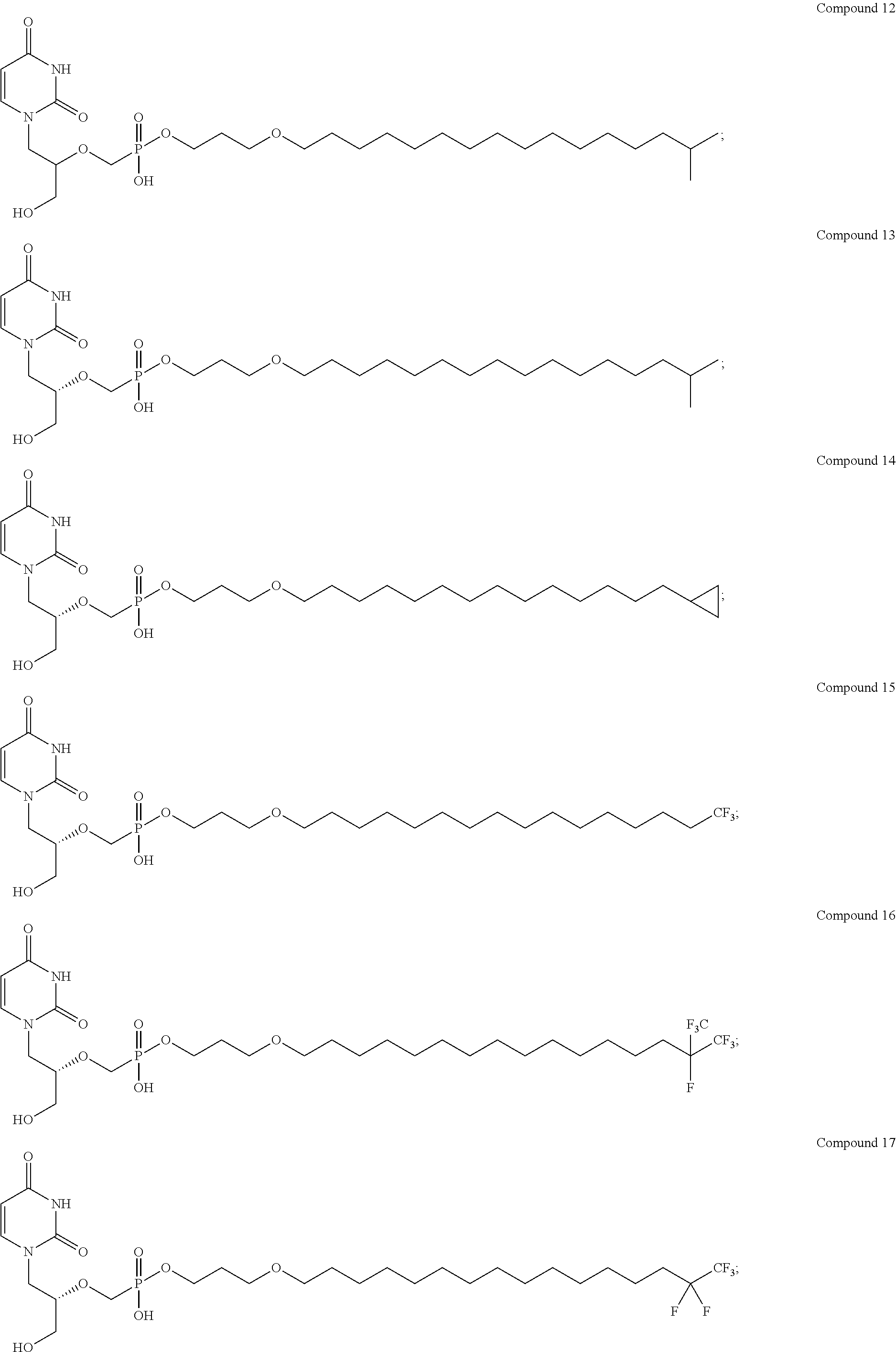

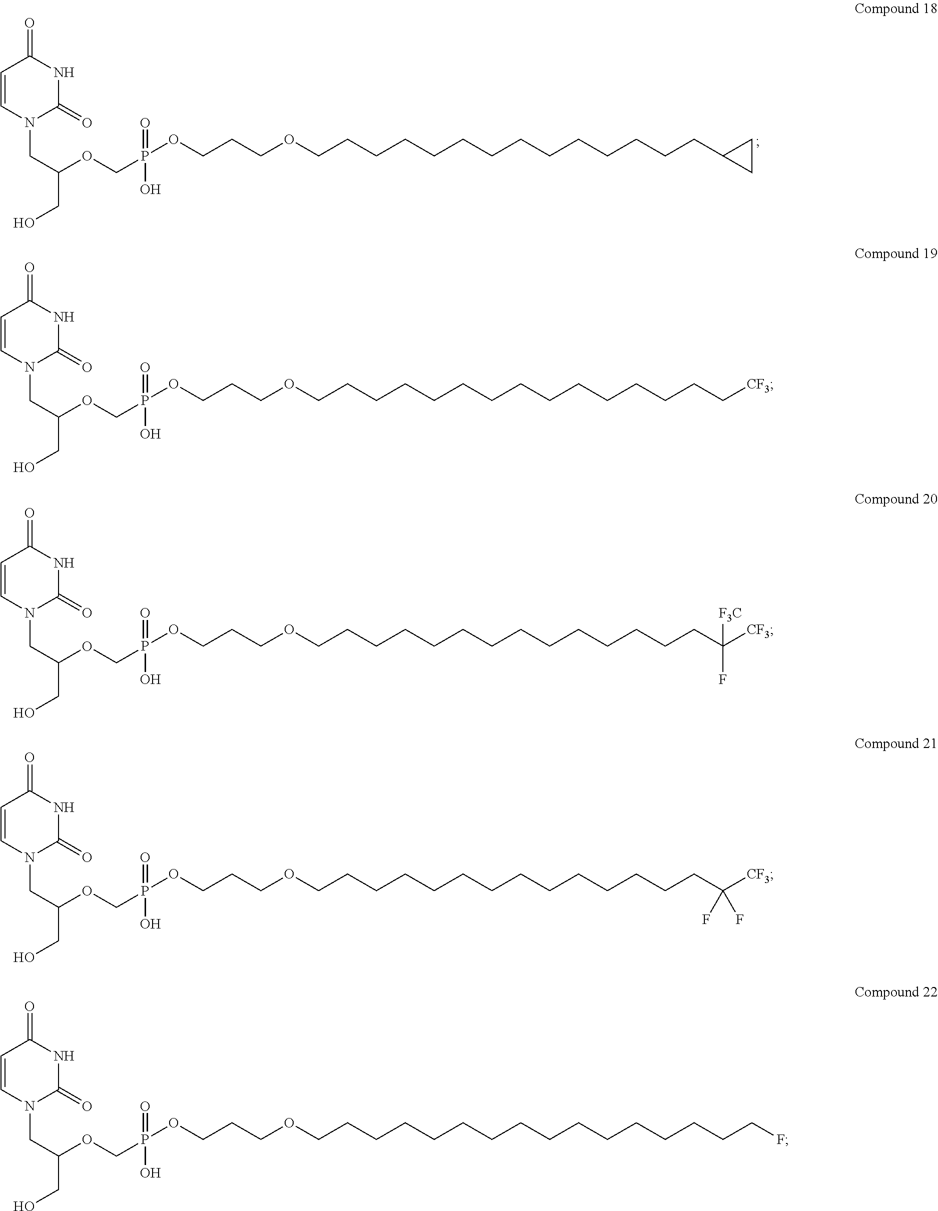

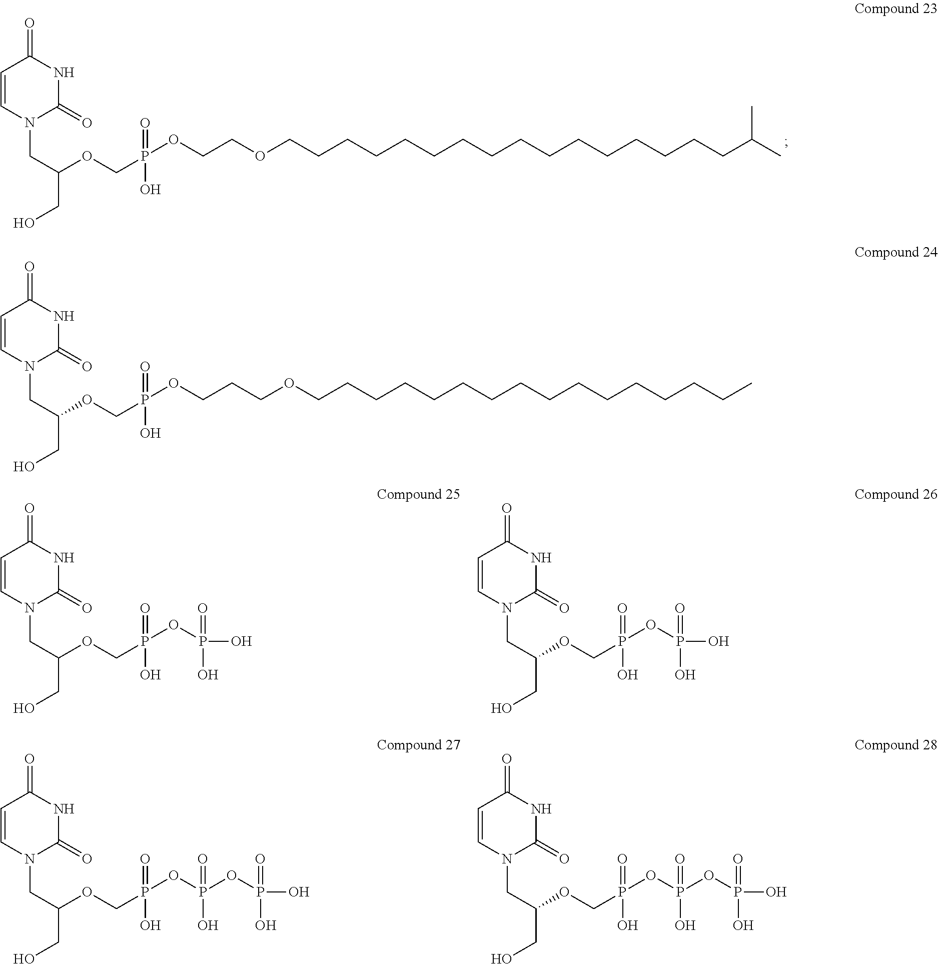

and pharmaceutically acceptable salts thereof.

One of the embodiments of the present invention relates to a method of synthesizing a representative compound of Formula (I):

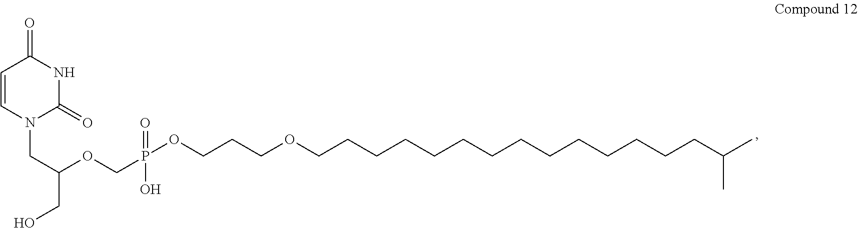 [SMILES: CC(C)CCCCCCCCCCCCCCOCCCOP(=O)(O)COC(CO)Cn1ccc(=O)[nH]c1=O]

as described in Scheme A below.

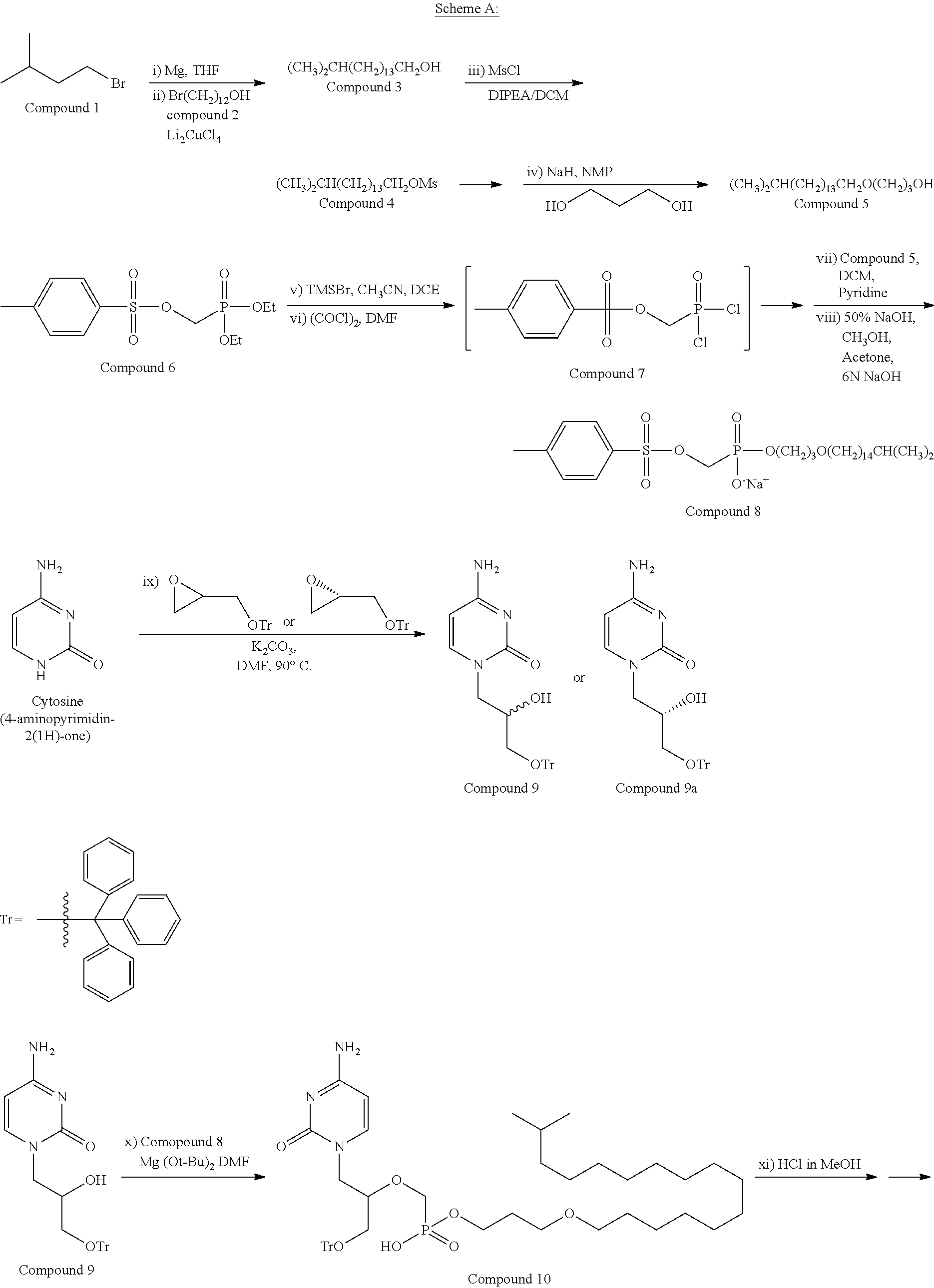

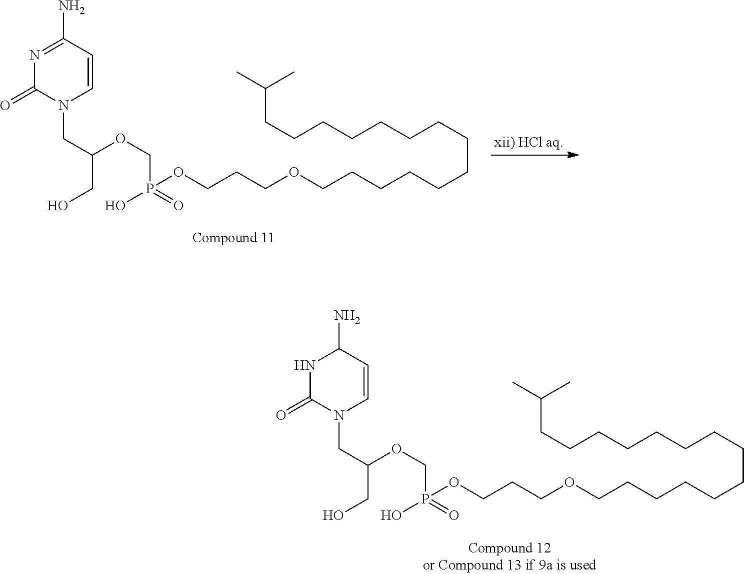 [SMILES: CC(C)CCCCCCCCCCCCCCOCCCOP(=O)(O)COC(CO)Cn1ccc(N)nc1=O ; CC(C)CCCCCCCCCCCCCCOCCCOP(=O)(O)COC(CO)CN1C=CC(N)NC1=O]

For example, the method includes the following:
(i) adding magnesium turnings to a solution of 1-bromo-3-methyl butane in 2-methyltetrahydrofuran (Me-THF);
(ii) adding 12-bromo-1-dodecanol in Me-THF to the reaction mixture of step followed by dilithium tetrachlorocuprate solution in tetrahydrofuran (THF) to produce Compound 3;
(iii) adding mesyl chloride to a cold solution of Compound 3 and N,N-Diisopropylethylamine (DIPEA) in dichloromethane while maintaining the temperature below about 5° C. to provide Compound 4;
(iv) adding sodium hydride (NaH) to a cold solution of 1,3-propane diol in N-Methyl-2-pyrrolidone (NMP) followed by addition of Compound 4 to produce Compound 5;
(v) adding trimethylsilyl bromide (TMS-Br) to a solution of Compound 6 (commercially available from Lacamas Laboratories) in acetonitrile;
(vi) adding oxalyl chloride and Dimethylformamide (DMF) after the removal of acetonitrile and TMS-Br to form Compound 7;
(vii) adding pyridine to the solution of Compound 7 and Compound 5 in dichloromethane;
(viii) adjusting the pH of an isolated intermediate to about 9.0 to produce Compound 8;
(ix) adding potassium carbonate to a mixture of cytosine and (S)-trityl glycidyl ether in anhydrous N,N-dimethylformamide and heating to about 90° C. provided Compound 9 (in racemic or enantiomerically pure form);
(x) adding magnesium di-tert butoxide to a mixture of Compound 8 and Compound 9 in DMF to produce Compound 10;
(xi) treating Compound 10 with HCl (methanol or organic solvent substitute) to generate compound 11;
(xii) adding water to Compound 11 and heating to about 90-100° C. to provide Compound 12.

The present invention also relates to a pharmaceutical formulation of the compounds of the present invention for use in a method for treating or preventing a viral infection or viral infection associated disease or disorder, e.g., a double stranded DNA (dsDNA) viral infection.

The present invention also relates to use of the pharmaceutical formulation of the invention in the manufacture of a medicament for treating or preventing a viral infection and/or viral infection associated disease or disorder, e.g., a dsDNA viral infection.

The present invention also relates to methods for treating or preventing a viral infection and/or viral infection associated disease or disorder, e.g., a dsDNA viral infection.

Unless otherwise defined, all technical and scientific terms used herein have the same meaning as commonly understood by one of ordinary skill in the art to which this invention belongs. In the case of conflict, the present specification, including definitions, will control. In the specification, the singular forms also include the plural unless the context clearly dictates otherwise. Although methods and materials similar or equivalent to those described herein can be used in the practice or testing of the present invention, suitable methods and materials are described below. All publications, patent applications, patents, and other references mentioned herein are incorporated by reference. The references cited herein are not admitted to be prior art to the claimed invention. In addition, the materials, methods, and examples are illustrative only and are not intended to be limiting.

Other features and advantages of the present invention will be apparent from the following detailed description and claims.

### DETAILED DESCRIPTION OF THE INVENTION

The present disclosure provides compounds, pharmaceutical compositions, and methods of synthesizing and using the compounds for treating or preventing a viral infection or viral infection associated disease or disorder, e.g., a dsDNA viral infection.

The compounds of the present disclosure have improved efficacy/toxicity ratio compared to compounds in the art used similarly.

### DEFINITIONS

For purposes of the present invention, the following definitions will be used (unless expressly stated otherwise):

The term “a compound of the invention” or “compounds of the invention” refers to a compound(s) disclosed herein e.g., a compound(s) of the invention includes a compound(s) of any of the Compounds represented by Formula (I) disclosed herein. Whenever the term is used in the context of the present invention it is to be understood that the reference is being made to the free base and the corresponding pharmaceutically acceptable salts thereof, provided that such is possible and/or appropriate under the circumstances. It is understood that Compounds 12-24 described herein are subsets of the compounds of Formula (I).

The term “alkyl,” as used herein, refers to saturated, straight- or branched-chain hydrocarbon radicals containing, in certain embodiments, between one and six, or one and eight carbon atoms, respectively. Branched means that one or more lower C₁-C₆ alkyl groups such as methyl, ethyl or propyl are attached to a linear alkyl chain. Exemplary alkyl groups include methyl, ethyl, n-propyl, i-propyl, nbutyl, t-butyl, n-pentyl, and 3-pentyl. Examples of C₁-C₆ alkyl radicals include, but are not limited to, methyl, ethyl, propyl, isopropyl, butyl, tert-butyl, neopentyl, n-hexyl radicals; and examples of C₁-C₈ alkyl radicals include, but are not limited to, methyl, ethyl, propyl, isopropyl, n-butyl, tert-butyl, neopentyl, n-hexyl, heptyl, octyl radicals.

The term “alkenyl,” as used herein, denotes a monovalent group derived from a hydrocarbon moiety containing, in certain embodiments, from two to six, or two to eight carbon atoms having at least one carbon-carbon double bond. The double bond may or may not be the point of attachment to another group. Examples of C₂-C₈ alkenyl groups include, but are not limited to, for example, ethenyl, propenyl, butenyl, 1-methyl-2-buten-1-yl, heptenyl, octenyl and the like.

The term “alkoxy” refers to an —O-alkyl radical.

The term “aryl,” as used herein, refers to a mono- or poly-cyclic carbocyclic ring system having one or more aromatic rings, fused or non-fused, including, but not limited to, phenyl, naphthyl, tetrahydronaphthyl, indanyl, idenyl and the like. The term aryl includes indoline.

The term “cycloalkyl,” as used herein, denotes a monovalent group derived from a monocyclic or polycyclic saturated or partially unsatured carbocyclic ring compound. Examples of C₃-C₈-cycloalkyl (3- to 8-membered cycloalkyl) include, but not limited to, cyclopropyl, cyclobutyl, cyclopentyl, cyclohexyl, cyclopentyl and cyclooctyl; and examples of C₃-C₁₂-cycloalkyl include, but not limited to, cyclopropyl, cyclobutyl, cyclopentyl, cyclohexyl, bicyclo [2.2.1] heptyl, and bicyclo [2.2.2] octyl. Also contemplated is a monovalent group derived from a monocyclic or polycyclic carbocyclic ring compound having at least one carbon-carbon double bond by the removal of a single hydrogen atom. Examples of such groups include, but are not limited to, cyclopropenyl, cyclobutenyl, cyclopentenyl, cyclohexenyl, cycloheptenyl, cyclooctenyl, and the like.

The term “heteroaryl,” as used herein, refers to a mono- or poly-cyclic (e.g., bi-, or tri-cyclic or more) fused or non-fused, radical or ring system having at least one aromatic ring, having from five to ten ring atoms of which one ring atoms is selected from S, O and N; zero, one or two ring atoms are additional heteroatoms independently selected from S, O and N; and the remaining ring atoms are carbon.

The term “5- or 6-membered heteroaryl” is taken to mean a ring having five or six ring atoms of which one ring atom is selected from S, O, and N. Heteroaryl includes, but is not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, and the like.

The term “3- to 8-membered heterocyclic” as used herein, refers to a non-aromatic 3-, 4-, 5-, 6-7- or 8-membered ring or a bi- or tri-cyclic group fused of non-fused system, where (i) each ring contains between one and three heteroatoms independently selected from oxygen, sulfur and nitrogen, (ii) each 5-membered ring has 0 to 1 double bonds and each 6-membered ring has 0 to 2 double bonds, (iii) the nitrogen and sulfur heteroatoms may optionally be oxidized, (iv) the nitrogen heteroatom may optionally be quaternized, and (iv) any of the above rings may be fused to a benzene ring. Representative heterocycloalkyl groups include, but are not limited to, [1,3]dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

In accordance with the invention, any of the aryls, substituted aryls, heteroaryls and substituted heteroaryls described herein, can be any aromatic group. Aromatic groups can be substituted or unsubstituted.

The terms “hal,” “halo,” and “halogen,” as used herein, refer to an atom selected from fluorine, chlorine, bromine and iodine.

Substituted or Unsubstituted: As described herein, compounds of the invention may optionally be substituted with one or more substituents, such as are illustrated generally above, or as exemplified by particular classes, subclasses, and species of the invention. It will be appreciated that the phrase “optionally substituted” is used interchangeably with the phrase “substituted or unsubstituted.” In general, the term “substituted,” whether preceded by the term “optionally” or not, refers to the replacement of hydrogen radicals in a given structure with the radical of a specified substituent. Unless otherwise indicated, an optionally substituted group may have a substituent at each substitutable position of the group, and when more than one position in any given structure may be substituted with more than one substituents selected from a specified group, the substituent may be either the same or different at every position.

The term “pharmaceutical” or “pharmaceutically acceptable” when used herein as an adjective, means substantially non-toxic and substantially non-deleterious to the recipient. As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

By “pharmaceutical formulation” it is further meant that the carrier, solvent, excipient(s) and salt must be compatible with the active ingredient of the formulation (e.g. a compound of the invention). It is understood by those of ordinary skill in this art that the terms “pharmaceutical formulation” and “pharmaceutical composition” are generally interchangeable, and they are so used for the purposes of this application and include preparations suitable for administration to mammals, e.g., humans.

A “pharmaceutical composition” as used herein relates to a formulation containing a compound of the present invention in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this invention include powders, sprays, ointments, pastes, creams, lotions, gels, solutions, patches and inhalants. In one embodiment, the active compound is mixed under sterile conditions with a pharmaceutically acceptable carrier, and with any preservatives, buffers or propellants that are required.

As used herein, “pharmaceutically acceptable carrier” may include any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the compounds such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogen free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. “Pharmaceutically acceptable excipient or carrier” also relates to an excipient or carrier that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

Some of the compounds of the present invention may exist in unsolvated as well as solvated forms such as, for example, hydrates.

“Solvate” means a solvent addition form that contains either a stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate. In the hydrates, the water molecules are attached through secondary valencies by intermolecular forces, in particular hydrogen bridges. Solid hydrates contain water as so-called crystal water in stoichiometric ratios, where the water molecules do not have to be equivalent with respect to their binding state. Examples of hydrates are sesquihydrates, monohydrates, dihydrates or trihydrates. Equally suitable are the hydrates of salts of the compounds of the invention.

The invention also includes metabolites of the compounds described herein. Metabolites from chemical compounds, whether inherent or pharmaceutical, are formed as part of the natural biochemical process of degrading and eliminating the compounds. The rate of degradation of a compound is an important determinant of the duration and intensity of its action. Profiling metabolites of pharmaceutical compounds, drug metabolism, is an important part of drug discovery, leading to an understanding of any undesirable side effects.

Physiologically/Pharmaceutically Acceptable/Compatible Salts: Physiologically acceptable, i.e. pharmaceutically compatible, salts can be salts of the compounds of the invention with inorganic or organic acids. Preference is given to salts with inorganic acids, such as, for example, hydrochloric acid, hydrobromic acid, phosphoric acid or sulphuric acid, or to salts with organic carboxylic or sulphonic acids, such as, for example, acetic acid, trifluoroacetic acid, propionic acid, maleic acid, fumaric acid, malic acid, citric acid, tartaric acid, lactic acid, benzoic acid, or methanesulphonic acid, ethanesulphonic acid, benzenesulphonic acid, toluenesulphonic acid or naphthalenedisulphonic acid.

Other pharmaceutically compatible salts which may be mentioned are salts with customary bases, such as, for example, alkali metal salts (for example sodium or potassium salts), alkaline earth metal salts (for example calcium or magnesium salts) or ammonium salts, derived from ammonia or organic amines, such as, for example, diethylamine, triethylamine, ethyldiisopropylamine, procaine, dibenzylamine, N-methylmorpholine, dihydroabietylamine or methylpiperidine.

As used herein, “pharmaceutically acceptable salts” refer to derivatives of the compounds of the present invention wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present invention also encompasses salts formed when an acidic proton present in the parent compound either is replaced by a metal ion, e.g., an alkali metal ion, or an alkaline earth metal ion, e.g., an aluminum ion; or coordinates with an organic base such as ethanolamine, diethanolamine, triethanolamine, tromethamine, N-methylglucamine, diethylamine, diethylaminoethanol, ethylenediamine, imidazole, lysine, arginine, morpholine, 2-hydroxyethylmorpholine, dibenzylethylenediamine, trimethylamine, piperidine, pyrrolidine, benzylamine, tetramethylammonium hydroxide and the like.

As used herein, the term “treat,” “treating,” or “treatment” herein, is meant decreasing the symptoms, markers, and/or any negative effects of a condition in any appreciable degree in a patient who currently has the condition. In some embodiments, treatment may be administered to a subject who exhibits only early signs of the condition for the purpose of decreasing the risk of developing the disease, disorder, and/or condition.

As used herein, the term “prevent,” “prevention,” or “preventing” refers to any method to partially or completely prevent or delay the onset of one or more symptoms or features of a disease, disorder, and/or condition. Prevention treatment may be administered to a subject who does not exhibit signs of a disease, disorder, and/or condition.

The term “therapeutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

As used herein, “subject” means a human or animal (in the case of an animal, more typically a mammal). In one aspect, the subject is a human. In one aspect, the subject is a male. In one aspect, the subject is a female.

It should be understood that all references to pharmaceutically acceptable salts include solvent addition forms (solvates) or crystal forms (polymorphs) as defined herein, of the same salt.

The compounds of the present invention can also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., acetate, propionate, or other esters.

U.S. Pat. No. 7,749,983, the entire content of which is incorporated herein by reference in its entirety, relates to esters of phosphonates, nucleoside phosphonate or nucleoside phosphate compounds, including terminal or penultimate branched chain, unsaturated and halogen substituted alkoxyalkyl esters of phosphonate compounds. In some other embodiments Compounds of Formula (I) can incorporate sidechains which are disclosed as R-groups in U.S. Pat. No. 7,749,983.

The present invention includes new compounds generally represented by Formula (I), or pharmaceutically acceptable salts thereof, and methods for preparation and uses thereof.

The compounds of the present invention can also be prepared prodrugs. In certain embodiments, one or more compounds of the present invention are formulated as a prodrug. In certain embodiments, upon in vivo administration, a prodrug is chemically converted to the biologically, pharmaceutically or therapeutically more active form. In certain embodiments, prodrugs are useful because they are easier to administer than the corresponding active form. For example, in certain instances, a prodrug may be more bioavailable (e.g., through oral administration) than is the corresponding active form. In certain instances, a prodrug may have improved solubility compared to the corresponding active form. In certain embodiments, prodrugs are less water soluble than the corresponding active form. In certain instances, such prodrugs possess superior transmittal across cell membranes, where water solubility is detrimental to mobility. In certain embodiments, a prodrug is an ester. In certain such embodiments, the ester is metabolically hydrolyzed to carboxylic acid upon administration. In certain instances the carboxylic acid containing compound is the corresponding active form. In certain embodiments, a prodrug comprises a short peptide (polyaminoacid) bound to an acid group. In certain of such embodiments, the peptide is cleaved upon administration to form the corresponding active form.

In certain embodiments, a prodrug is produced by modifying a pharmaceutically active compound such that the active compound will be regenerated upon in vivo administration. The prodrug can be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

The compounds, or pharmaceutically acceptable salts, esters or derivatives thereof, are administered orally, nasally, transdermally, pulmonary, inhalationally, buccally, sublingually, intraperintoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter or arrest the progress of the condition.

### Compounds of the Invention

The nucleoside phosphonates of the instant invention can also be generally represented by Formula (I) as follows:

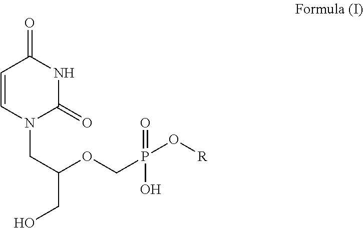

wherein R is:

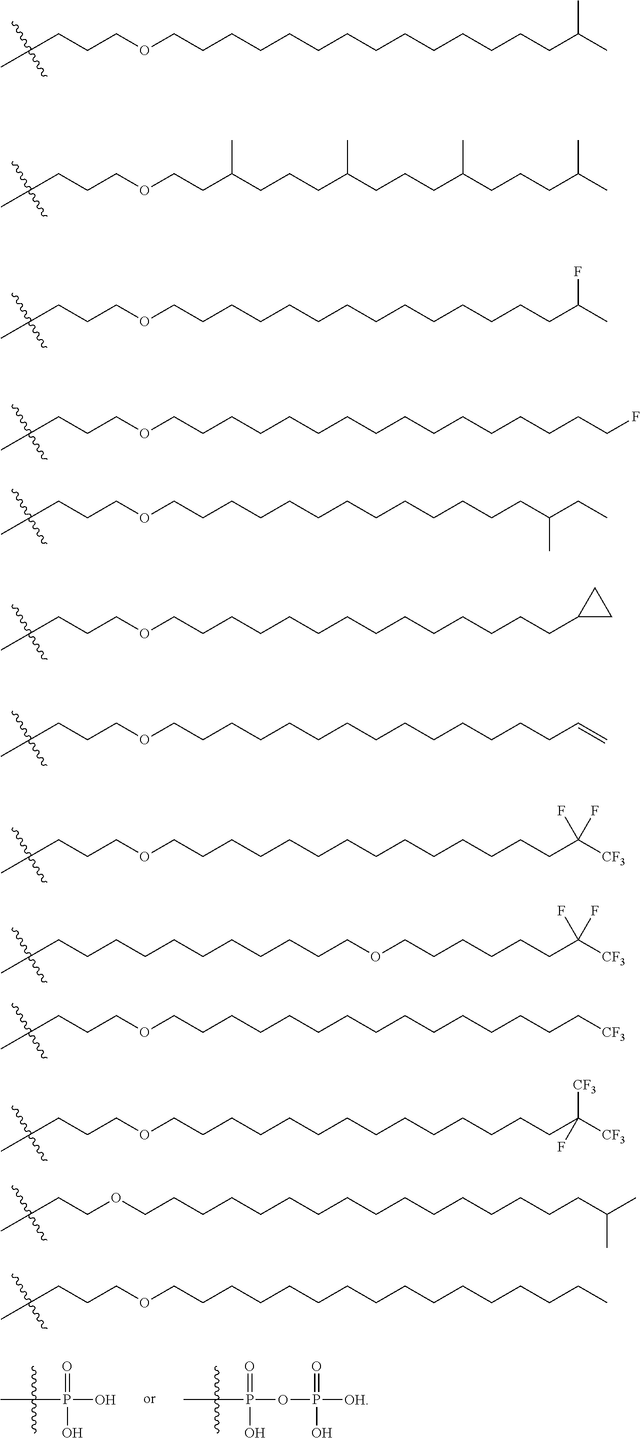

Representative nucleoside phosphonates of the instant invention are listed in Table 1.

TABLE 1

| Compound | Representative Non-limiting Examples of Compounds of |
|---|---|
| No. | the Present Disclosure |
| 12 | 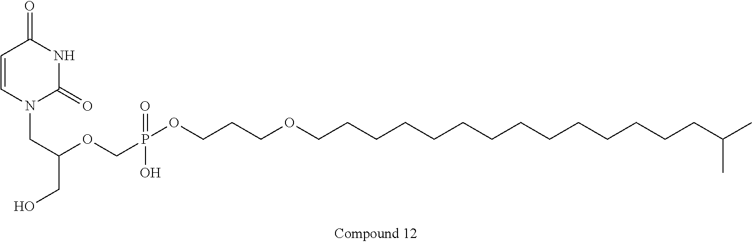 [SMILES: CC(C)CCCCCCCCCCCCCCOCCCOP(=O)(O)COC(CO)Cn1ccc(=O)[nH]c1=O] |
| 13 | 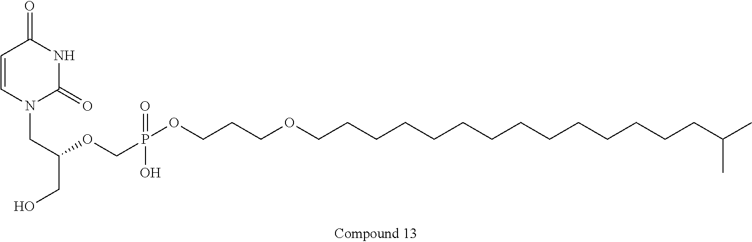 [SMILES: CC(C)CCCCCCCCCCCCCCOCCCOP(=O)(O)CO[C@H](CO)Cn1ccc(=O)[nH]c1=O] |
| 14 | 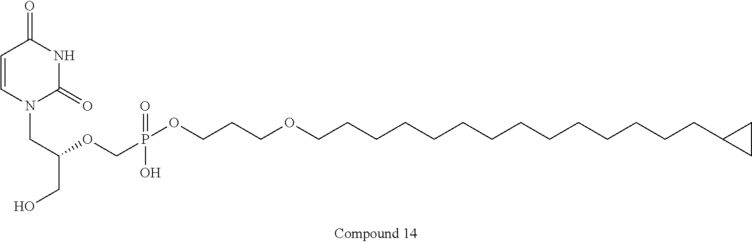 [SMILES: O=c1ccn(C[C@@H](CO)OCP(=O)(O)OCCCOCCCCCCCCCCCCCCC2CC2)c(=O)[nH]1] |
| 15 | 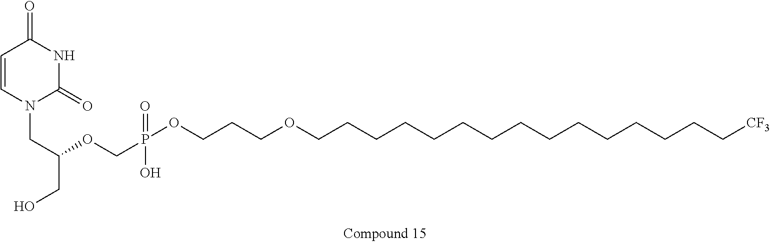 [SMILES: O=c1ccn(C[C@@H](CO)OCP(=O)(O)OCCCOCCCCCCCCCCCCCCCC(F)(F)F)c(=O)[nH]1] |
| 16 | 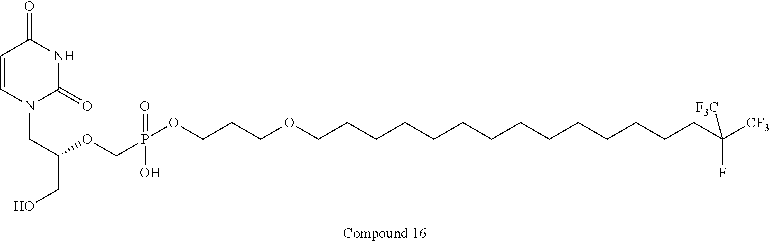 [SMILES: O=c1ccn(C[C@@H](CO)OCP(=O)(O)OCCCOCCCCCCCCCCCCCCC(F)(C(F)(F)F)C(F)(F)F)c(=O)[nH]1] |
| 17 | 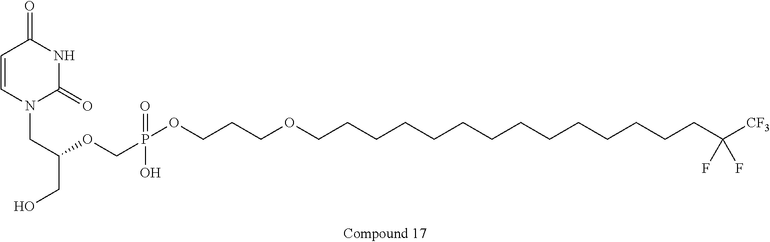 [SMILES: O=c1ccn(C[C@@H](CO)OCP(=O)(O)OCCCOCCCCCCCCCCCCCCC(F)(F)C(F)(F)F)c(=O)[nH]1] |
| 18 | 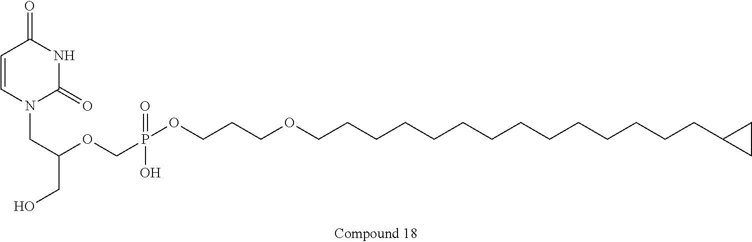 [SMILES: O=c1ccn(CC(CO)OCP(=O)(O)OCCCOCCCCCCCCCCCCCCC2CC2)c(=O)[nH]1] |
| 19 | 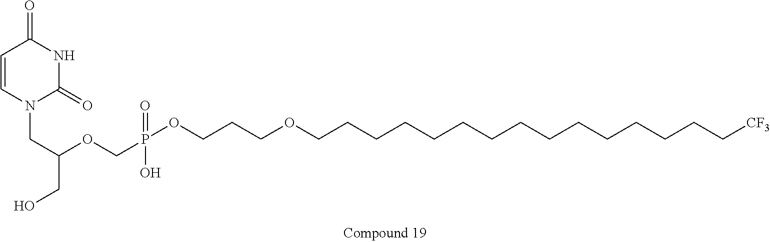 [SMILES: O=c1ccn(CC(CO)OCP(=O)(O)OCCCOCCCCCCCCCCCCCCCC(F)(F)F)c(=O)[nH]1] |
| 20 | 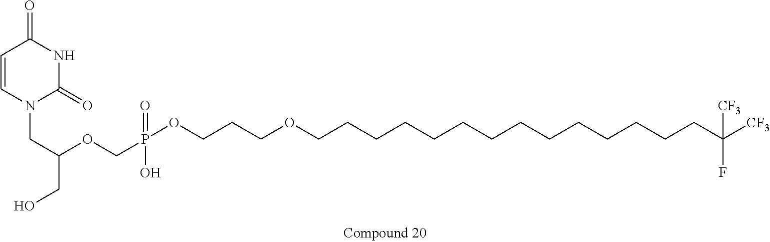 [SMILES: O=c1ccn(CC(CO)OCP(=O)(O)OCCCOCCCCCCCCCCCCCCC(F)(C(F)(F)F)C(F)(F)F)c(=O)[nH]1] |
| 21 | 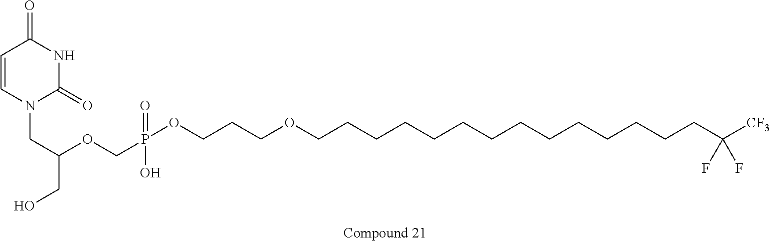 [SMILES: O=c1ccn(CC(CO)OCP(=O)(O)OCCCOCCCCCCCCCCCCCCC(F)(F)C(F)(F)F)c(=O)[nH]1] |
| 22 | 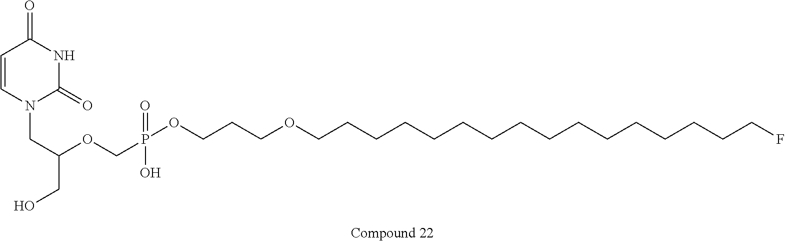 [SMILES: O=c1ccn(CC(CO)OCP(=O)(O)OCCCOCCCCCCCCCCCCCCCCF)c(=O)[nH]1] |
| 23 | 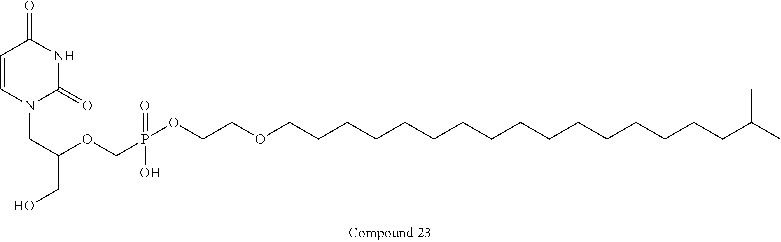 [SMILES: CC(C)CCCCCCCCCCCCCCCCOCCOP(=O)(O)COC(CO)Cn1ccc(=O)[nH]c1=O] |
| 24 | 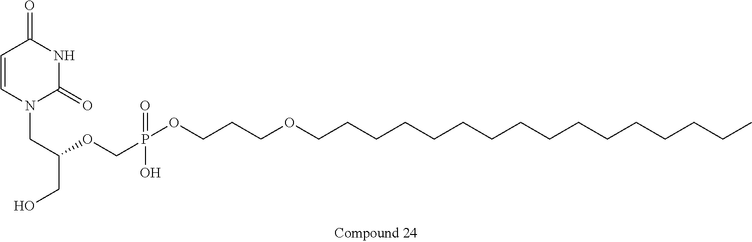 [SMILES: CCCCCCCCCCCCCCCCOCCCOP(=O)(O)CO[C@H](CO)Cn1ccc(=O)[nH]c1=O] |
| 25 | 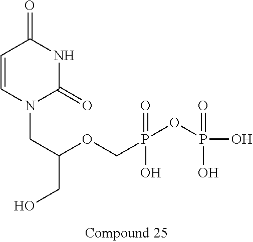 [SMILES: O=c1ccn(CC(CO)OCP(=O)(O)OP(=O)(O)O)c(=O)[nH]1] |
| 26 | 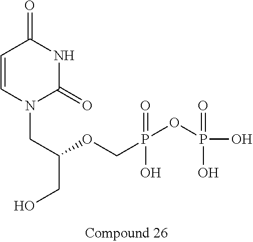 [SMILES: O=c1ccn(C[C@@H](CO)OCP(=O)(O)OP(=O)(O)O)c(=O)[nH]1] |
| 27 | 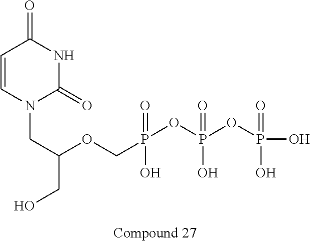 [SMILES: O=c1ccn(CC(CO)OCP(=O)(O)OP(=O)(O)OP(=O)(O)O)c(=O)[nH]1] |
| 28 | 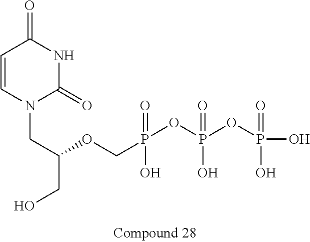 [SMILES: O=c1ccn(C[C@@H](CO)OCP(=O)(O)OP(=O)(O)OP(=O)(O)O)c(=O)[nH]1] |

In additional embodiments, the present disclosure provides Compound 8 and a method of synthesizing Compound 8 having formula:

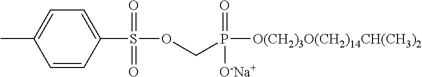

wherein the method includes the following steps:
(i) adding magnesium turnings to a solution of 1-bromo-3-methyl butane in 2-methyltetrahydrofuran (Me-THF), followed by heating and then cooling the mixture;
(ii) adding 12-bromo-1-dodecanol in Me-THF to the reaction mixture of step (i), and adding immediately thereafter dilithium tetrachlorocuprate solution in tetrahydrofuran (THF);
(iii) back extracting the aqueous phase of the reaction mixture with ethyl acetate;
(iv) washing the organic solution with brine and drying over MgSO₄, filtering, and then concentrating in vacuo to produce Compound 3;
(v) adding mesyl chloride to a cold solution of Compound 3 and N,N-Diisopropylethylamine (DIPEA) in dichloromethane while maintaining the temperature below about 5° C.;
(vi) warming the reaction to room temperature and stirring for several hours;
(vii) adding mesyl chloride and DIPEA to the reaction mixture and stirring for several more hours;
(viii) adding water while cooling the reaction mixture, then separating the dichloromethane (DCM) layer from the aqueous layer, drying over a drying agent, and filtering the DCM layer to remove the drying agent;
(ix) concentrating the DCM solution in vacuo to give a yellow oil; and adding methanol to the concentrate of the yellow oil;
(x) filtering the concentrate in methanol precipitating a white solid, filtering the solid and drying to yield Compound 4;
(xi) adding sodium hydride (NaH) to a cold solution of 1,3-propane diol in N-Methyl-2-pyrrolidone (NMP), and warming the mixture;
(xii) adding Compound 4 to the solution at step (xi) and stirring for several hours;
(xiii) adding water and ethyl acetate to the solution and separting the organic layer;
(xiv) concentrtaing the organic layer in vacuo, adding methanol and then drying;
(xv) adding acetonitrile, repeating step (xiv), forming Compound 5 after filtering and drying;
(xvi) adding trimethylsilyl bromide (TMS-Br) to a solution of Compound 6 (obtained commercially from Lacamas Laboratories) in acetonitrile;
(xvii) adding dichloromethane after removing acetonitrile and TMS-Br and then concentrating;
(xviii) adding oxalyl chloride and Dimethylformamide (DMF) and concentrating in vacuo to form Compound 7;
(xix) mixing Compound 5 and Compound 7 in dichloromethane, and adding pyridine to the solution of Compound 7 and Compound 5;
(xx) separating the organic layer after adding water to the mixture in step (xix);
(xxi) separating the organic layer again after adding water and methanol to the organic layer in step (xx);
(xxii) drying the organic layer from step (xxi) in vacuo, and adding acetone;
(xxiii) drying and adding acetone before further drying and adjusting the pH to about 9.0;
(xxiv) adding acetone after filtering solid formed in step (xxiii); and
(xxv) filtering and drying the solid product from step (xxiv) to produce Compound 8.

In another embodiment the method includes the following steps:
(i) adding magnesium turnings to a solution of 1-bromo-3-methyl butane (or appropriately substituted hydrocarbon) in 2-methyltetrahydrofuran (Me-THF);
(ii) adding 12-bromo-1-dodecanol (or appropriately substituted hydrocarbon) in Me-THF to the reaction mixture of step followed by dilithium tetrachlorocuprate solution in tetrahydrofuran (THF) to produce Compound 3;
(iii) adding mesyl chloride to a cold solution of Compound 3 and N,N-Diisopropylethylamine (DIPEA) in dichloromethane while maintaining the temperature below about 5° C. to provide Compound 4;
(iv) adding sodium hydride (NaH) to a cold solution of 1,3-propane diol in N-Methyl-2-pyrrolidone (NMP) followed by addition of Compound 4 to produce Compound 5;
(v) adding trimethylsilyl bromide (TMS-Br) to a solution of Compound 6 (commercially available from Lacamas Laboratories) in acetonitrile;
(vi) adding oxalyl chloride and Dimethylformamide (DMF) after the removal of acetonitrile and TMS-Br to form Compound 7;
(vii) adding pyridine to the solution of Compound 7 and Compound 5 in dichloromethane;
(viii) adjusting the pH of an isolated intermediate to about 9.0 to produce Compound 8.

Compounds 12-24 synthesized following the method of the present disclosure are substantially free of impurities. Compounds 12-24 synthesized following the method of the present embodiment are more than or equal to about 99% w/w pure. It will be appreciated that the methods disclosed herein may be suitable for both large-scale and small-scale preparations of the desired compounds. In preferred embodiments of the methods described herein, the phosphonate esters may be prepared on a large scale, for example on an industrial production scale rather than on an experimental/laboratory scale. For example, a batch-type process according to the methods of the disclosure allows the preparation of batches of at least 1 g, or at least 5 g, or at least 10 g, or at least 100 g, or at least 1 kg, or at least 100 kg of phosphonate ester product. The compounds of the present invention may be prepared as enantiomers, diastereomers, and racemates. Furthermore, the methods allow the preparation of a phosphonate ester product having a purity of at least 98%, or at least 98.5% as measured by HPLC. In preferred embodiments according to the disclosure, these products are obtained in a reaction sequence that does not involve purification by any form of chromatography (e.g., gas chromatography, HPLC, preparative LC, size exclusion chromatography, and the like).

In one embodiment the present disclosure relates to Compound 12:

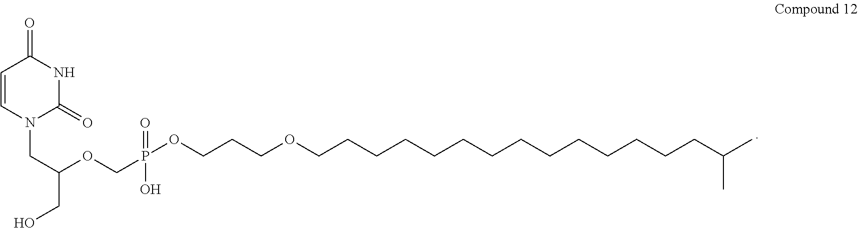 [SMILES: CC(C)CCCCCCCCCCCCCCOCCCOP(=O)(O)COC(CO)Cn1ccc(=O)[nH]c1=O]

In one embodiment the present disclosure relates to Compound 13:

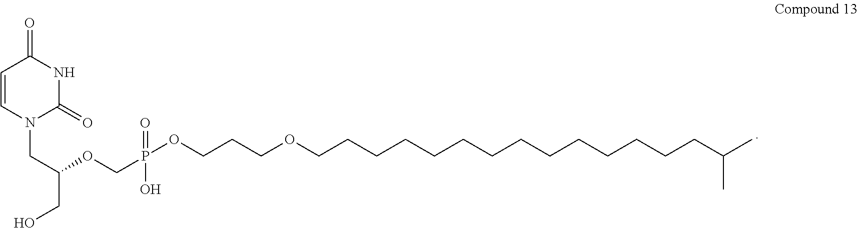 [SMILES: CC(C)CCCCCCCCCCCCCCOCCCOP(=O)(O)CO[C@H](CO)Cn1ccc(=O)[nH]c1=O]

In another embodiment, one or both of the hydrogens in one or more of the —CH₂ groups of the side chain of Compound 12 and/or Compound 13 are optionally substituted with alkyl, halogen, or any other group as disclosed in U.S. Pat. No. 7,749,983.

In some embodiments, the terminal —CH₃ is substituted with halogen or alkyl or any other group as disclosed in U.S. Pat. No. 7,749,983. For example, one embodiment of the present invention relates to Compound 22, where a terminal CH₃ group is replaced with a fluorine group:

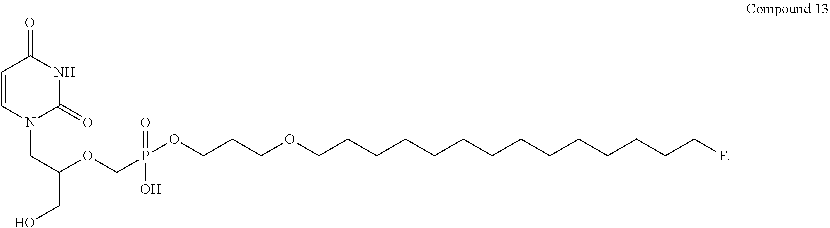 [SMILES: O=c1ccn(CC(CO)OCP(=O)(O)OCCCOCCCCCCCCCCCCCCF)c(=O)[nH]1]

### Methods of Synthesis

The present disclosure provides methods of synthesis for branched nucleoside phosphonic acid esters. In certain aspects, the invention provides methods for the preparation of compounds generally represented by Formula (I):

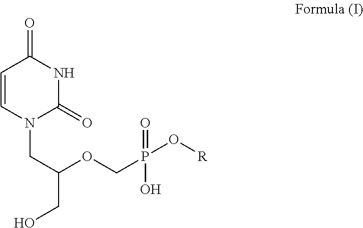

wherein R is:

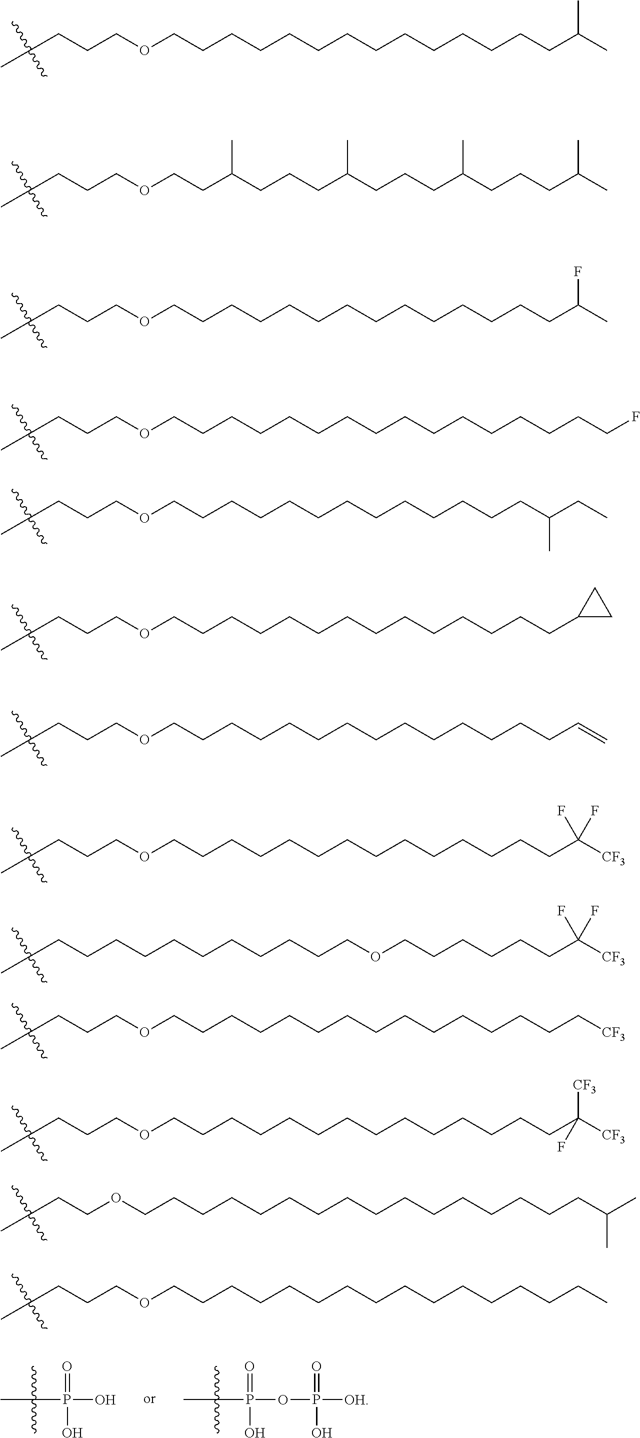

In one embodiment, the present disclosure provides methods for the preparation of representative Compounds listed in Table 1.

The embodiments of the present disclosure provide methods of synthesis of compounds of Formula (I) or pharmaceutically acceptable salts or solvates thereof. In one embodiment the present disclosure provides a method of synthesis of Compound 12 and/or Compound 13. The present disclosure also provides a method of synthesis of Compound 8.

The present disclosure provides methods of synthesis for substituted phosphonic acid esters. In certain aspects, the invention provides methods for the preparation of Compound 12 having the structure:

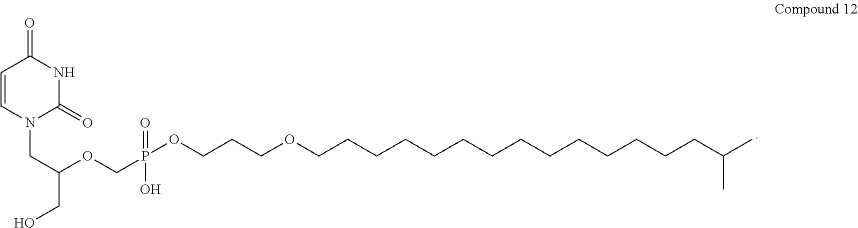 [SMILES: CC(C)CCCCCCCCCCCCCCOCCCOP(=O)(O)COC(CO)Cn1ccc(=O)[nH]c1=O]

In one embodiment, the present disclosure provides methods for the preparation of Compound 13 having the structure:

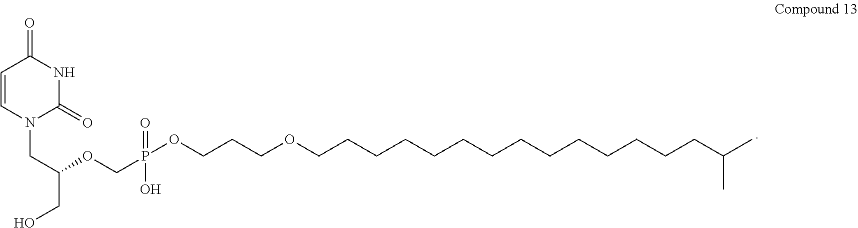 [SMILES: CC(C)CCCCCCCCCCCCCCOCCCOP(=O)(O)CO[C@H](CO)Cn1ccc(=O)[nH]c1=O]

or enantiomer, diastereomer, racemate or a mixture thereof, or a pharmaceutically acceptable salts thereof.

The present disclosure provides intermediate Compound 8 and method of synthesis thereof

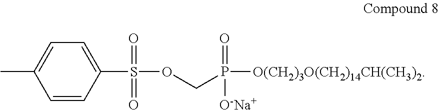

The scheme and description below depicts some methods for the preparation of a compound of the invention.

The synthetic processes of the invention can tolerate a wide variety of functional groups; therefore various substituted starting materials can be used. The processes generally provide the desired final compound at or near the end of the overall process, although it may be desirable in certain instances to further convert the compound to a pharmaceutically acceptable salt, ester or derivative thereof

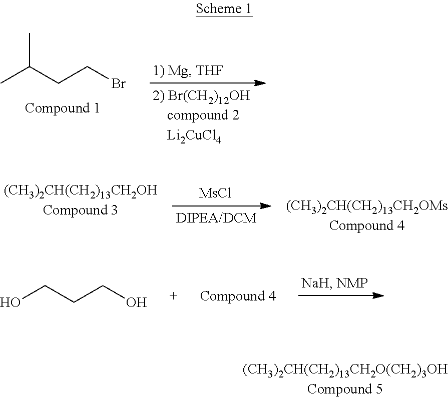

Synthesis of Compound 3: Compound 3 of the present disclosure is synthesized by adding alkaline earth metal, e.g. magnesium in form of turnings, to a solution 1-bromo-3-methyl butane in a solvent, e.g., 2-Methyltetrahydrofuran (Me-THF). To prevent increase in temperature a dry ice and ketone (e.g., acetone (propanone)) bath is used. At about 30-60° C., e.g., about 40° C., halogen, e.g., a small chip of iodine is added. The solution is then heated to about 60-80° C., e.g., about 61° C., and stirred for about 1-3 hours (e.g., about 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0 hours). The mixture is cooled to about 30-60° C., e.g., equal to or less than about 40° C. The reaction mixture is further cooled to about 0 to −70° C., e.g., about −59° C. An alcohol, e.g., a fatty alcohol such as 12-bromo-1-dodecanol, in an organic solvent, e.g., Me-THF, was added to the mixture. Immediately following the addition of the an alcohol, e.g., a fatty alcohol such as 12-bromo-1-dodecanol, a catalyst solution, e.g., dilithium tetrachlorocuprate(II) solution, is added. The reaction is warmed to about room temperature and stirred for several hours, e.g., about 16 hours. Upon completion of the reaction, the reaction is cooled to about 0° C. and an inorganic solvent, e.g., NH₄Cl, is slowly added until saturation and until the temperature increased to about 15-30° C. e.g., above about 22° C. The aqueous phase is back extracted with an organic additive, e.g., ethyl acetate. The combined organics is washed with a salt solution, e.g., brine, dried over an inorganic salt, e.g., MgSO₄, and then filtered. The solution is concentrated in vacuo at about 40° C.

Synthesis of Compound 4: To a cold solution of Compound 3 and an organic reagent that is a strong base but a poor nucleophile, e.g., N,N-Diisopropylethylamine (DIPEA), in a solvent, e.g., dichloromethane, mesyl chloride is added slowly to ensure the temperature does not increase above about 5° C. To the reaction mixture containing Compound 3, a mesylating agent, e.g., mesyl chloride (methanesulfonyl chloride) and DIPEA is added. Water is added after cooling the mixture and stirred. The dichloromethane (DCM) layer is separated, dried over Na₂SO₄, and filtered. The solution is concentrated in vacuo at about 40-50° C. To the yellow residue methanol is added. The solution was left at about 4° C. for about 30 minutes to precipitate a white solid. The solution with precipitated solid is filtered, and the solid is air dried on filter for several hours. The filtrate is concentrated to one-half volume and filtered. The solids are combined and triturated in methanol. The white solid is filtered and dried.

Synthesis of Compound 5: To a cold solution, less than 0° C., e.g., about −5° C., to 1,3-propane diol, a suitable organic reagent, e.g., N-Methyl-2-pyrrolidone (NMP), and a strong base, e.g., NaH, is added. This mixture is warmed to room temperature and stirred for about 30 minutes. To this solution, compound 4 dissolved in a suitable organic reagent, e.g., NMP, is added. To the reaction mixture water and an organic solvent, e.g., ethyl acetate, are added. The organic and aqueous layers are separated. The organic layer is washed with water. The solution is concentrated in vacuo at about 40° C. The mixture is dried further by adding methanol and concentrated in vacuo at about 40° C. The step of drying and concentrating in methanol is repeated with a polar aprotic solvent, e.g., acetonitrile. To the yellow oil a polar aprotic solvent, e.g., acetonitrile is added and this mixture is stirred. A waxy white solid is formed, which is filtered. It is dried on the rotary evaporator to provide Compound 5.

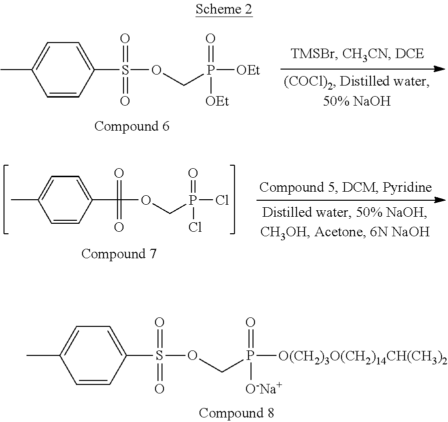

Synthesis of Compound 8: To a solution of compound 6 in a polar aprotic solvent, e.g., acetonitrile, trimethylsilyl bromide (TMS-Br) is added. After the addition is complete, the internal temperature is adjusted to about 55° C. After the mixture is stirred for about 2 hours, the polar aprotic solvent, e.g., acetonitrile, and TMS-Br are removed via vacuum distillation at about 40° C. to form a concentrate. To the concentrate, an organic solvent, e.g., dichloromethane, is added to form a solution followed by. a suitable organic reagent, e.g., oxalyl chloride, After the addition of the organic reagent, e.g., oxalyl chloride, is complete, an organic solvent, e.g., polar (hydrophilic) aprotic solvent (for example, Dimethylformamide (DMF)) is added. The reaction mixture is stirred and then concentrated in vacuo at an external temperature of about 35° C. to provide Compound 7. Intermediate Compound 7 may be used for the next step without purification.

The solution of Compound 5 and Compound 7 (0.1462 mol, 44.33 g) in dichloromethane (423 ml) is cooled below 0° C., e.g., about −8° C. Pyridine (0.381 mol, 30.18 g) is added to the cooled solution. This reaction mixture is stirred for 3 hours. Thin Layer Chromatography (TLC) in 2:1 hexanes:ethyl acetate is performed, which indicates that Compound 5 has been consumed. To the reaction mixture (cooled to 10° C.) 200 ml water is added. This mixture is stirred for 0.5 hr. The organic layer is then separated. To the organic layer 100 ml water and 75 ml methanol are added. The organic layer is again separated and concentrated in vacuo at 35° C. To the residue 200 ml acetone is added and the pH is adjusted to about 9.04 using 6N NaOH (˜15 ml used). This mixture is left at about 4° C. for about 16 hours. 10 g of a white solid precipitates after the incubation period. The mixture with white solid precipitate is filtered. To the mixture an additional 300 ml acetone is added. The mixture is again left at 4° C. for 16 hours. Solid of tan color precipitates after the additional incubation period. The mixture is filtered and dried to produce Compound 8.

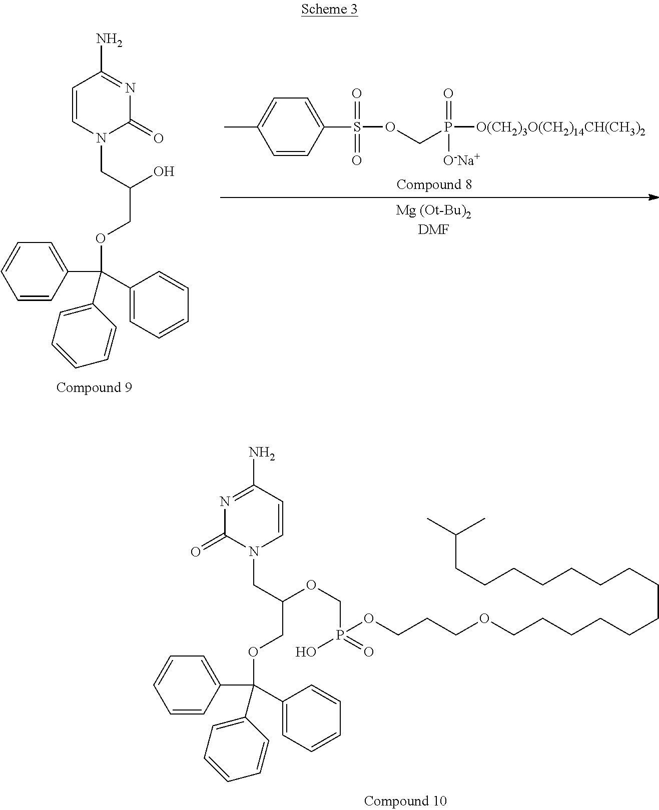

Synthesis of Compound 10: Compound 9, Compound 8, a mild alkaline earth metal alkoxide (base), e.g., magnesium di-tert butoxide, and DMF (25 ml) are heated together to about 60° C. or more, e.g., about 80° C. for more than 1 hour, e.g., about 3 hours. The reaction is cooled to room temperature in an organic solvent, e.g., isopropyl acetate, and acid, e.g., HCl was added. The organic layer is separated, washed with a salt solution, e.g., brine, dried over a salt, e.g., Na₂SO₄, and concentrated in vacuo at about 40° C. To the mixture methanol is added, which is concentrated to provide Compound 10.

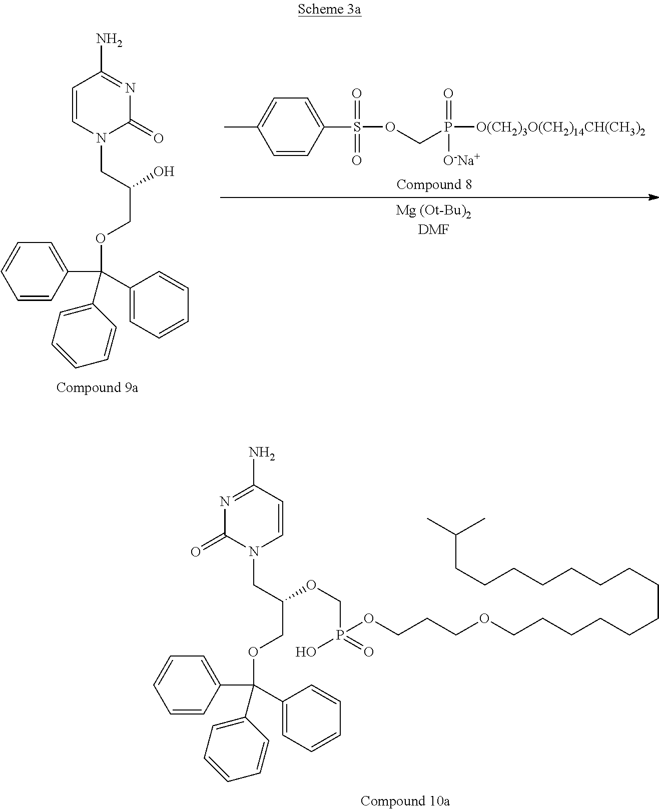

Synthesis of Compound 10a: Compound 9a, Compound 8, a mild alkaline earth metal alkoxide (base), e.g., magnesium di-tert butoxide, and DMF (25 ml) are heated together to about 60° C. or more, e.g., about 80° C. for more than 1 hour, e.g., about 3 hours. The reaction is cooled to room temperature in an organic solvent, e.g., isopropyl acetate, and acid, e.g., HCl was added. The organic layer is separated, washed with a salt solution, e.g., brine, dried over a salt, e.g., Na₂SO₄, and concentrated in vacuo at about 40° C. To the mixture methanol is added, which is concentrated to provide Compound 10a.

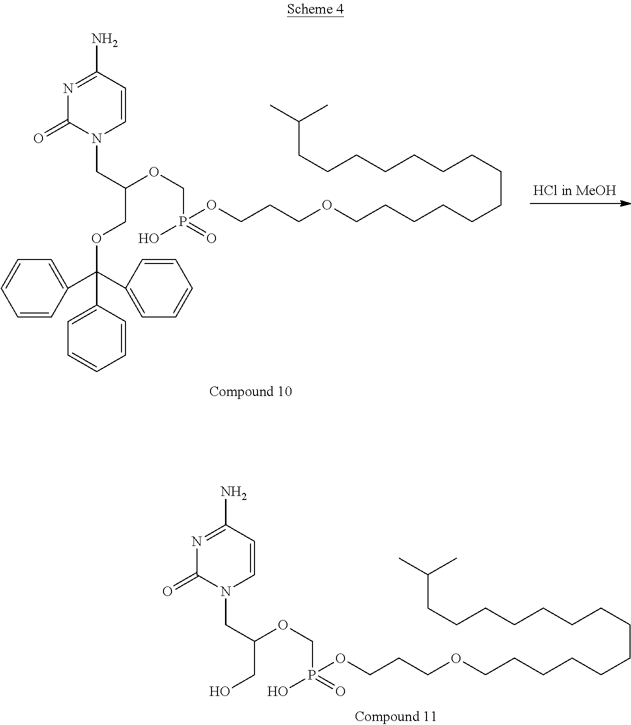 [SMILES: CC(C)CCCCCCCCCCCCCCOCCCOP(=O)(O)COC(COC(c1ccccc1)(c1ccccc1)c1ccccc1)Cn1ccc(N)nc1=O ; CC(C)CCCCCCCCCCCCCCOCCCOP(=O)(O)COC(CO)Cn1ccc(N)nc1=O]

Synthesis of Compound 11: Alcohol, e.g., methanol, and acid, e.g., HCl, are added to Compound 10 at room temperature. The white solid, a trityl byproduct, is formed and filtered off. The filtrate is diluted with water and the pH is adjusted to about 2.5 using an alkali metal hydroxide, e.g., NaOH, forming Compound 11. The product is slurried in an organic solvent, e.g., acetone (propanone), and filtered. The product is dried in the vacuum oven at room temperature.

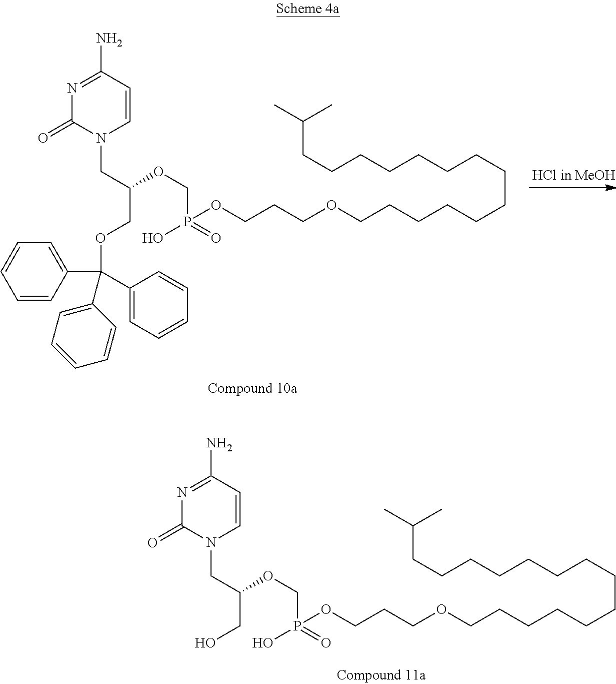 [SMILES: CC(C)CCCCCCCCCCCCCCOCCCOP(=O)(O)CO[C@H](COC(c1ccccc1)(c1ccccc1)c1ccccc1)Cn1ccc(N)nc1=O ; CC(C)CCCCCCCCCCCCCCOCCCOP(=O)(O)CO[C@H](CO)Cn1ccc(N)nc1=O]

Synthesis of Compound 11a: Alcohol, e.g., methanol, and acid, e.g., HCl, are added to Compound 10a at room temperature. The white solid, a trityl byproduct, is formed and filtered off. The filtrate is diluted with water and the pH is adjusted to about 2.5 using an alkali metal hydroxide, e.g., NaOH, forming Compound 11a. The product is slurried in an organic solvent, e.g., acetone (propanone), and filtered. The product is dried in the vacuum oven at room temperature.

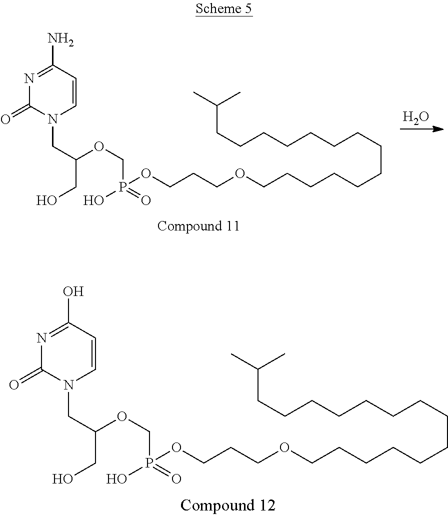

Synthesis of Compound 12: Water is added to Compound 11 before the mixture is heated to about 90° C. and stirred. After the reaction is equal to or more than 95% complete, the mixture is cooled to room temperature. The pH is adjusted to about 1-2 using an acid, e.g., HCl. The aqueous solution is extracted with ethyl acetate. The combined ethyl acetate extracts are dried over Na₂SO₄, filtered, and concentrated in vacuo at about 40° C. to produce thick oil. This thick oil is triturated with acetone and cooled to produce a white solid. The white solid is filtered and dried to produce Compound 12.

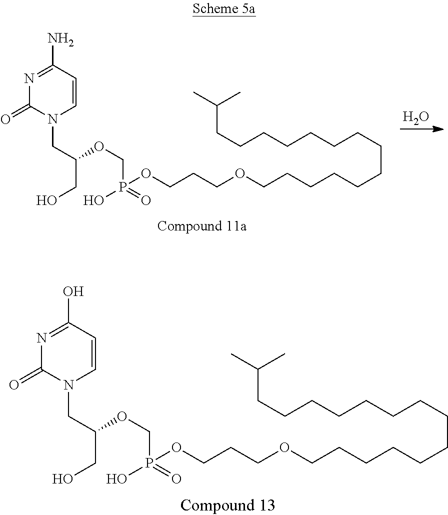

Synthesis of Compound 13: Water is added to Compound 11a before the mixture is heated to about 90° C. and stirred. After the reaction is equal to or more than 95% complete, the mixture is cooled to room temperature. The pH is adjusted to about 1-2 using an acid, e.g., HCl. The aqueous solution is extracted with ethyl acetate. The combined ethyl acetate extracts are dried over Na₂SO₄, filtered, and concentrated in vacuo at about 40° C. to produce thick oil. This thick oil is triturated with acetone and cooled to produce a white solid. The white solid is filtered and dried to produce Compound 13.

A similar method is used to prepare other Compounds of Formula (I). Starting materials, such as Compound 1 and/or Compound 3 may vary as necessary for the synthesis of compounds of the present invention.

Throughout the description, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

### Purity of Compounds

The present disclosure provides compounds of Formula (I), e.g., any one of Compounds 12-24 (or pharmaceutically acceptable salts thereof) having more than or equal to about 91% w/w, more than or equal to about 95% w/w, or more than or equal to about 99% w/w purity.

In some embodiments, compounds represented by Formula (I), e.g., Compounds 12-24, of the present disclosure are substantially free of impurities. In some embodiments, the purity of the Compounds 12-24 or pharmaceutically acceptable salts thereof is equal to or greater than 92% (e.g., ≧92%, ≧93%, ≧94%, ≧95%, ≧96%, ≧97%, ≧98%, ≧99%, or ≧99.5%). In yet other embodiments, Compounds 12-24 or pharmaceutically acceptable salts thereof have a purity of equal to or greater than 91% (e.g., ≧91%, ≧92%, ≧93%, ≧94%, ≧95%, ≧96%, ≧97%, ≧98%, >99%, or >99.5%). In yet another embodiment, Compounds 12-24 or pharmaceutically acceptable salts thereof are solvates, e.g., a methanol solvate, an ethanol solvate, or an isopropanol solvate.

In some embodiments, the purity of the compounds represented by Formula (I), e.g., Compounds 12-24, or pharmaceutically acceptable salts thereof is equal to or greater than 92% (e.g., ≧92%, ≧93%, ≧94%, ≧95%, ≧96%, ≧97%, ≧98%, ≧99%, or ≧99.5%). In yet other embodiments, any one of the Compounds 12-24 (or a pharmaceutically acceptable salt thereof) has a purity of equal to or greater than 91% (e.g., ≧91%, ≧92%, ≧93%, ≧94%, ≧95%, ≧96%, ≧97%, ≧98%, >99%, or >99.5%). In one embodiment, the purity of any one of the Compounds 12-24 (or a pharmaceutically acceptable salt thereof) is about 99%.

The present disclosure also provides for the use of the compounds of Formula (I) (or pharmaceutically acceptable salts thereof), e.g., any one of the Compounds 12-24 or pharmaceutically acceptable salt thereof, having a purity of equal to or greater than 91% w/w, e.g., having less than or equal to 9% w/w of impurities, in the manufacture of a medicament for the therapeutic and/or prophylactic treatment of viral infection in a subject, e.g., an immunodeficient subject.

The present disclosure provides more than about 99% w/w pure compounds represented by Formula (I), e.g., Compounds 12-24, or a pharmaceutically acceptable salt thereof. In some embodiments, the any one of the compounds represented by Formula (I), e.g., Compounds 12-24, or a pharmaceutically acceptable salt thereof is equal to or more than about 99% w/w, 98% w/w, 97% w/w, 96% w/w, 95% w/w, 94% w/w, 93% w/w, 92% w/w or 91% w/w, pure.

### Pharmaceutical Compositions

Throughout the description, where compositions are described as having, including, or comprising specific components, it is contemplated that compositions also consist essentially of, or consist of, the recited components.

In another aspect, provided herein are pharmaceutical compositions comprising compounds of Formula (I) or pharmaceutically acceptable salts thereof. In some embodiments, the present disclosure provides pharmaceutical compositions comprising compounds of Formula (I) or pharmaceutically acceptable salts thereof and a pharmaceutically acceptable carrier and/or diluent. The pharmaceutical compositions of the present disclosure comprises any one of the Compounds 12-24 or pharmaceutically acceptable salt thereof. The present disclosure also provides pharmaceuticllay compositions comprising any one of the Compounds 12-24 or pharmaceutically acceptable salt thereof and a pharmaceutically acceptable carrier and/or diluent.

Techniques for formulation and administration of the disclosed compounds of the invention can be found in Remington: the Science and Practice of Pharmacy, 19^{th} edition, Mack Publishing Co., Easton, Pa. (1995).

In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

In another embodiment, the disclosure provides a method for the therapeutic and/or prophylactic treatment of viral infection in a subject, e.g., an immunodeficient subject, the method comprising administering any one of the Compounds 12-24 or pharmaceutically acceptable salt thereof having a purity of equal to or greater than 91% w/w, e.g., having less than or equal to 9% w/w of impurities, to the subject.

The present disclosure provides, compounds of Formula (I) (or pharmaceutically acceptable salts thereof), e.g., any one of the Compounds 12-24 or a pharmaceutically acceptable salt thereof, having a purity of equal to or greater than 91% for use in treating a viral infection (e.g., a dsDNA viral infection) in a subject. The viral infections include ut are not limited to human cytomegalovirus (HCMV), BK virus (BKV), Epstein-Barr virus (EBV), adenovirus, JC virus (JCV), SV40, MC virus (MCV), KI virus (KIV), WU virus (WUV), vaccinia, herpes simplex virus 1 (HSV-1), herpes simplex virus 2 (HSV-2), human herpes virus 6 (HHV-6), human herpes virus 8 (HHV-8), hepatitis B virus, hepatitis C virus, varicella zoster virus (VZV), variola major, variola minor, smallpox, cowpox, camelpox, monkeypox, poliovirus, ebola virus, Marburg virus, enterovirus, papilloma virus, and human immunodeficiency virus (HIV) infections. For example, the infection is resistant to valganciclovir hydrochloride (or ganciclovir) or where the subject exhibits side effects to valganciclovir hydrochloride (or ganciclovir). Alternatively or additionally, compounds of Formula (I) (or pharmaceutically acceptable salts thereof), e.g., any one of the Compounds 12-24 or a pharmaceutically acceptable salt thereof, having a purity of equal to or greater than 91% w/w is used to treat CMV. For example, human cytomegalovirus (HCMV). For example, infection is subsequent to treatment with ganciclovir, for example, where the CMV infection is emergent. The patient may be a stem cell transplant patient, e.g., a bone marrow stem cell transplant patient, especially where there is a risk (real or perceived) for bone marrow toxicity from ganciclovir in the patient.

In another embodiment, a compound of Formula (I) or pharmaceutically acceptable salt thereof having a purity of equal to or greater than about 91% is administered orally to a subject, for example, at a dosage of about 0.01 mg/kg to about 10 mg/kg or more, e.g., up to 100 mg/kg. In another embodiment, a compound of Formula (I) or pharmaceutically acceptable salt thereof having a purity of equal to or greater than about 91% w/w is administered to a subject at a dosage of about 0.01 mg/kg, 0.05 mg/kg, 0.1 mg/kg, 0.5 mg/kg, 1 mg/kg, 1.5 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, or 10 mg/kg or more or any range therein.

In some embodiments, compounds of Formula (I) (or pharmaceutically acceptable salts thereof), e.g., any one of the Compounds 12-24 of the present disclosure, is administered to a subject at a dose of about 1-20 mg/kg (e.g., about 1-1.1 mg/kg, about 1.1-1.2 mg/kg, about 1.2-1.3 mg/kg, about 1.3-1.4 mg/kg, about 1.4-1.5 mg/kg, about 1.5-1.6 mg/kg, about 1.6-1.7 mg/kg, about 1.7-1.8 mg/kg, about 1.8-1.9 mg/kg, about 1.9-2.0 mg/kg, about 2.0-2.1 mg/kg, about 2.1-2.2 mg/kg, about 2.2-2.3 mg/kg, about 2.3-2.4 mg/kg, about 2.4-2.5 mg/kg, about 2.5-2.6 mg/kg, about 2.6-2.7 mg/kg, about 2.7-2.8 mg/kg, about 2.8-2.9 mg/kg, about 2.9-3.0 mg/kg, about 3.0-3.1 mg/kg, about 3.1-3.2 mg/kg, about 3.2-3.3 mg/kg, about 3.3-3.4 mg/kg, about 3.4-3.5 mg/kg, about 3.5-3.6 mg/kg, about 3.6-3.7 mg/kg, about 3.7-3.8 mg/kg, about 3.8-3.9 mg/kg, 3.9-4.0 mg/kg, about 4.0-5.0 mg/kg, about 5.0-6.0 mg/kg, about 6.0-7.0 mg/kg, about 7.0-8.0 mg/kg, about 8.0-9.0 mg/kg, about 9.0-10.0 mg/kg, or about 10-20 mg/kg).

In some embodiments, compounds of Formula (I) (or pharmaceutically acceptable salts thereof), e.g., any one of the Compounds 12-24 of the present disclosure is used in the manufacture of a medicament for administration to a subject at a dose of about 1-20 mg/kg (e.g., about 1-1.1 mg/kg, about 1.1-1.2 mg/kg, about 1.2-1.3 mg/kg, about 1.3-1.4 mg/kg, about 1.4-1.5 mg/kg, about 1.5-1.6 mg/kg, about 1.6-1.7 mg/kg, about 1.7-1.8 mg/kg, about 1.8-1.9 mg/kg, about 1.9-2.0 mg/kg, about 2.0-2.1 mg/kg, about 2.1-2.2 mg/kg, about 2.2-2.3 mg/kg, about 2.3-2.4 mg/kg, about 2.4-2.5 mg/kg, about 2.5-2.6 mg/kg, about 2.6-2.7 mg/kg, about 2.7-2.8 mg/kg, about 2.8-2.9 mg/kg, about 2.9-3.0 mg/kg, about 3.0-3.1 mg/kg, about 3.1-3.2 mg/kg, about 3.2-3.3 mg/kg, about 3.3-3.4 mg/kg, about 3.4-3.5 mg/kg, about 3.5-3.6 mg/kg, about 3.6-3.7 mg/kg, about 3.7-3.8 mg/kg, about 3.8-3.9 mg/kg, about 3.9-4.0 mg/kg, 4.1-4.2 mg/kg, 4.2-4.3 mg/kg, 4.3-4.4 mg/kg, 4.4-4.5 mg/kg, 4.5-4.6 mg/kg, 4.6-4.7 mg/kg, 4.7-4.8 mg/kg, 4.8-4.9 mg/kg, about 4.9-5.0 mg/kg, about 5.0-6.0 mg/kg, about 6.0-7.0 mg/kg, about 7.0-8.0 mg/kg, about 8.0-9.0 mg/kg, about 9.0-10.0 mg/kg, or about 10-20 mg/kg).

In another embodiment, the disclosure also provides an oral dosage form comprising compounds of Formula (I) (or pharmaceutically acceptable salts thereof), e.g., any one of the Compounds 12-24 or a pharmaceutically acceptable salt thereof, having a purity of equal to or greater than 91% w/w, e.g., having less than or equal to 9% w/w impurities, for the therapeutic and/or prophylactic treatment of viral infection in a subject, wherein said oral dosage form, upon administration to a human at a dosage of about 2 mg/kg of said compound, provides an AUC of said compound of about 2000 to about 4000 h·ng/mL, e.g., about 2500 to about 3000 h·ng/mL.

In another embodiment, the disclosure also provides an oral dosage form comprising compounds of Formula (I) (or pharmaceutically acceptable salts thereof), e.g., any one of the Compounds 12-24 or a pharmaceutically acceptable salt thereof, having a purity of equal to or greater than about 91% w/w, e.g., having less than or equal to about 9% w/w impurities, for the therapeutic and/or prophylactic treatment of viral infection in a subject, wherein said oral dosage form, upon administration to a human at a dosage of about 1-2 mg/kg, about 2-3 mg/kg, about 3-4 mg/kg of said compound, provides a Cₘₐₓ of said compound of about 100 to about 500 ng/mL, e.g., about 200 to about 400 ng/mL.

In another embodiment, the disclosure also provides an oral dosage form comprising compounds of Formula (I) (or pharmaceutically acceptable salts thereof), e.g., any one of the Compounds 12-24 or a pharmaceutically acceptable salt thereof, having a purity of equal to or greater than about 91% w/w, e.g., having less than or equal to about 9% w/w impurities, for the therapeutic and/or prophylactic treatment of viral infection in a subject, wherein said oral dosage form, upon administration to a human at a dosage of about 1-2 mg/kg, about 2-3 mg/kg, about 3-4 mg/kg of said compound and metabolism of said compound to cidofovir, provides a Cₘₐₓ of said cidofovir that is less than about 30% of the Cₘₐₓ of said compound, e.g., less that about 20% of the Cₘₐₓ of said compound.

In some embodiments, the administration continues for ten total doses. For instance, the compounds of Formula (I) can be administered at dosages of about 100 mg twice a week for five weeks (i.e., ten total doses). Alternatively, the compounds of Formula (I) may be administered with a loading dose of about 200 mg followed by about 100 mg doses continuing twice a week. In some embodiments, the administration continues for ten total doses. For instance, the compounds of Formula (I) may be administered at a loading dose of about 200 mg followed by nine additional about 100 mg doses twice a week for a total of ten doses. In one of the embodiments of the present invention, Compounds of Formula (I) can be dosed daily in the range of about 20-200 mg/day or weekly in the range of about 200 mg-2000 mg.

When the compounds of the present invention are administered as pharmaceuticals to mammals, e.g., humans, they can be given per se or as a pharmaceutical composition containing, for example, about 0.1% to 99.9%, about 0.2 to 98%, about 0.3% to 97%, about 0.4% to 96%, or about 0.5 to 95% of active ingredient in combination with a pharmaceutically acceptable carrier. In one embodiment pharmaceutical composition containing about 0.5% to 90% of active ingredient in combination with a pharmaceutically acceptable carrier is suitable for administration to mammals, e.g., humans. Some embodiments of the present disclosure provide preparation of a pharmaceutical composition comprising about 0.1% to 99.9%, about 0.2 to 98%, about 0.3% to 97%, about 0.4% to 96%, or about 0.5 to 95% of the compounds of Formula (I) or pharmaceutically acceptable salts thereof, e.g., any one of the Compounds 12-24 or pharmaceutically acceptable salt thereof, for use in treating, preventing, or prophylaxis of viral infections or viral infection associated disorders. The present disclosure provides use of about 0.1% to 99.9%, about 0.2 to 98%, about 0.3% to 97%, about 0.4% to 96%, or about 0.5 to 95% of the compounds of Formula (I) or pharmaceutically acceptable salts thereof, e.g., any one of the Compounds 12-24 or a pharmaceutically acceptable salt thererof, for the manufacture of a medicament containing effective amounts of the compound for use in treating, preventing, or prophylaxis of viral infections and viral infection associated diseases.

The compounds of Formula (I) or pharmaceutically acceptable salts thereof, e.g., any one of the Compounds 12-24 or a pharmaceutically acceptable salt thererof, described herein may be combined with a pharmaceutically acceptable carrier according to conventional pharmaceutical compounding techniques. Furthermore, the carrier may take a wide variety of forms depending on the form of the preparation desired for administration, e.g. oral, nasal, rectal, vaginal, parenteral (including intravenous injections or infusions). In preparing compositions for oral dosage form any of the usual pharmaceutical media may be employed. Usual pharmaceutical media include, for example, water, glycols, oils, alcohols, flavoring agents, preservatives, coloring agents, and the like in the case of oral liquid preparations (such as for example, suspensions, solutions, emulsions and elixirs); aerosols; or carriers such as starches, sugars, microcrystalline cellulose, diluents, granulating agents, lubricants, binders, disintegrating agents and the like, in the case of oral solid preparations (such as for example, powders, capsules, and tablets).

Pharmaceutical compositions comprising the compounds of the present invention (e.g., compounds of Formula (I) may be formulated to have any concentration desired. In some embodiments, the composition is formulated such that it comprises at least a therapeutically effective amount. As used herein, “therapeutically effective amount” means that amount necessary to make a clinically observed improvement in the patient. In some embodiments, the composition is formulated such that it comprises an amount that would not cause one or more unwanted side effects.

Pharmaceutical compositions include those suitable for oral, sublingual, nasal, rectal, vaginal, topical, buccal and parenteral (including subcutaneous, intramuscular, and intravenous) administration, although the most suitable route will depend on the nature and severity of the condition being treated. The compositions may be conveniently presented in unit dosage form, and prepared by any of the methods well known in the art of pharmacy. In certain embodiments, the pharmaceutical composition is formulated for oral administration in the form of a pill, capsule, lozenge or tablet. In other embodiments, the pharmaceutical composition is in the form of a suspension.

The regimen of administration can affect what constitutes a pharmaceutically effective amount. The compounds of Formula (I) or pharmaceutically acceptable salts thereof, e.g., any one of the Compounds 12-24 or a pharmaceutically acceptable salt thererof, can be administered to the subject either prior to or after the onset of a disease. Further, several divided dosages, as well as staggered dosages can be administered daily or sequentially, or the dose can be continuously infused, or can be a bolus injection. Further, the dosages can be proportionally increased or decreased as indicated by the exigencies of the therapeutic or prophylactic situation. Further, the dosages may be co-administered in combination with other antiviral or, e.g., with chemotherapeutic agents known by the skilled artisan. Such agents include, but are not limited to Brincidofovir (BCV), ganciclovir (GCV), valganciclovir (vGCV), letermovir, and foscarnet and combinations thereof. For example, Compounds of Formula (I) can be used in combination with BCV, GCV, vGCV, letermovir, or foscarnet or combinations thereof to treat CMV infection and/or CMV-related disease or disorder. In another example, Compound of Formula (I) can be used in combination with BCV to treat BKV (BK virus) infection and/or BKV-related disease or disorder.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

In a preferred aspect, the disease or condition to be treated is viral infection.

For any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, once every two weeks, or monthly depending on half-life and clearance rate of the particular formulation.

In some embodiments, the administration continues for ten total doses. For instance, the compounds of Formula (I) can be administered at dosages of about 100 mg twice a week for five weeks (i.e., ten total doses). Alternatively, the compounds of Formula (I) may be administered with a loading dose of about 200 mg followed by about 100 mg doses continuing twice a week. In some embodiments, the administration continues for ten total doses. For instance, the compounds of Formula (I) may be administered at a loading dose of about 200 mg followed by nine additional about 100 mg doses twice a week for a total of ten doses. In one of the embodiments Compounds of Formula (I) can be dosed daily in the range of about 20-200 mg/day or weekly in the range of about 200 mg-2000 mg.

The pharmaceutical compositions containing compounds of Formula (I) of the present invention may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. The appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as, for example, peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved.

In therapeutic applications, the dosages of the pharmaceutical compositions used in accordance with the invention vary depending on the agent, the age, weight, and clinical condition of the recipient patient, and the experience and judgment of the clinician or practitioner administering the therapy, among other factors affecting the selected dosage. Dosages can range from about 0.01 mg/kg to about 100 mg/kg. In preferred aspects, dosages can range from about 0.1 mg/kg to about 10 mg/kg. In an aspect, the dose will be in the range of about 1 mg to about 1 g; about 10 mg to about 500 mg; about 20 mg to about 400 mg; about 40 mg to about 400 mg; or about 50 mg to about 400 mg, in single, divided, or continuous doses (which dose may be adjusted for the patient's weight in kg, body surface area in m², and age in years). In certain embodiments, the amount per dosage form can be about 0.1 mg to about 1000 mg, e.g., about 0.1 mg, about 0.5 mg, about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 200 mg, about 300 mg, about 400 mg, about 500 mg, about 600 mg, about 700 mg, about 800 mg, about 900 mg or about 1000 mg. In one embodiment, the amount can be about 20 mg. In one embodiment, the amount can be about 50 mg. In another embodimant the dosage can be 100 mg.

The compounds of Formula (I) or pharmaceutically acceptable salts thereof, e.g., any one of the Compounds 12-24 or a pharmaceutically acceptable salt thererof, of the present disclosure are formulated as a pharmaceutical composition or is used in the manufacture of a medicament for the treatment of a viral infection and/or viral infection associated disease and/or disorder. The composition and/or the medicament of the compounds of Formula (I) or pharmaceutically acceptable salts thereof, e.g., any one of the Compounds 12-24 or a pharmaceutically acceptable salt thererof, is formulated as a tablet or suspension. Tablets of the compounds of Formula (I) or pharmaceutically acceptable salts thereof, e.g., any one of the Compounds 12-24 or a pharmaceutically acceptable salt thererof, are formulated comprising pharmacologically acceptable buffers, excipients, carriers, including emulsifiers, enhancers (e.g., absorption enhancers), disintegrants (e.g., Polyvinylpolypyrrolidone (polyvinyl polypyrrolidone, PVPP, crospovidone, crospolividone or E1202), which is a highly cross-linked modification of polyvinylpyrrolidone (PVP)), and/or polymers disclosed in the present disclosure and well-known in the art.

In one embodiment, the present disclosure provides tablet formulation of the compounds of Formula (I) or pharmaceutically acceptable salts thereof, e.g., any one of the Compounds 12-24 or a pharmaceutically acceptable salt thererof, for use in prophylactic treatment or prevention viral infection and/or viral associated disease or disorder. The present disclosure provides tablet formulation of the compounds of Formula (I) or pharmaceutically acceptable salts thereof, e.g., any one of the Compounds 12-24 or a pharmaceutically acceptable salt thererof, for use in treating subjects in need of such treatment including but not limited to immunodeficient subjects, or pre- or post-organ and/or tissue transplantation subjects. The present disclosure provides the compounds of Formula (I) or pharmaceutically acceptable salts thereof, e.g., any one of the Compounds 12-24 or a pharmaceutically acceptable salt thererof, for the use in the manufacture of a medicament for use in treating subjects in need of such treatment including but not limited to immunodeficient subjects, or pre- or post-organ and/or tissue transplantation subjects.

In one embodiment, Compound 12 or Compound 13, or a pharmaceutically acceptable salt thereof, is formulated as a tablet for use in prophylactic treatment or prevention viral infection and/or viral associated disease or disorder. In some embodiments, Compound 12 or Compound 13, or a pharmaceutically acceptable salt thereof is formulated as a tablet for use in treating immunodeficient subjects, or pre- or post-organ and/or tissue transplantation subjects. In some embodiments, Compound 12 or Compound 13, or a pharmaceutically acceptable salt thereof is formulated as a tablet for the use in the manufacture of a medicament for use in treating subjects in need of such treatment including but not limited to immunodeficient subjects, or pre- or post-organ and/or tissue transplantation subjects.

In one embodiment, a 100 mg tablet formulation of the compounds of Formula (I) or pharmaceutically acceptable salts thereof, e.g., any one of the Compounds 12-24 or a pharmaceutically acceptable salt thererof, comprises silicified microcrystalline cellulose (Prosolv 90) (about 27.8% wt/tablet), crospovidone (Polyplasdone XL-10) (about 22% wt/tablet), microcrystalline cellulose and mannitol (Avicel HFE 102) (about 3.7% wt/tablet), microcrystalline cellulose (AVICEL® PHE102) about 11.4% wt/tablet), mannitol (Pearlitol 100 SD) about 33.9% wt/tablet), colloidal silicon dioxide (CAB-O-SIL®) (0.5% wt/tablet), and magnesium stearate (0.5% wt/tablet).

In one embodiment, the present disclosure provides suspension formulation of the compounds of Formula (I) or pharmaceutically acceptable salts thereof, e.g., any one of the Compounds 12-24 or a pharmaceutically acceptable salt thererof, for use in prophylactic treatment or prevention viral infection and/or viral associated disease and/or disorder. The present disclosure provides suspension formulation of the compounds of Formula (I) or pharmaceutically acceptable salts thereof, e.g., any one of the Compounds 12-24 or a pharmaceutically acceptable salt thererof, for use in treating subjects in need of such treatment including but not limited to immunodeficient subjects, or pre- or post-organ and/or tissue transplantation subjects.

In another embodiment of the present invention additional excipients include but are not limited to sodium phosphate, dibasic, citric acid (monohydrate) (about 0.06% wt), sodium citrate (about 0.10% wt), xanthum gum (about 0.04% wt), methylparaben (sodium salt) (about 0.17% wt), propylparaben (sodium salt) (about 0.02% wt), sucralose (about 0.05% wt), microcrystalline cellulose and carboxymethylcellulose sodium (VivaPur MCG 591) (about 1.56% wt), high fructose corn syrup (about 55% wt), lemon lime flavor (WONF220J15) (about 0.40% wt), sodium hydroxide pellets, sodium hydroxide/hydrochloric acid, and purified water (about 68.93% wt).

The formulations of the present disclosure are used in treating end-organ damage related to viral infection, e.g. treating, preventing, and/or ameliorating BKV infection associated end organ damage in a subject.

The formulations of the present disclosure are used in manufacturing a medicament in prophylactic treatment and/or prevention viral infection and/or viral associated disease and/or disorder.

In one embodiment, the compounds of Formula (I) or pharmaceutically acceptable salts thereof, e.g., any one of the Compounds 12-24 or a pharmaceutically acceptable salt thererof, is administered at a dose of about 100 mg (tablet or suspension formulation) twice a week. In some embodiments, the administration continues for ten total doses. For instance, the compounds of Formula (I) can be administered at dosages of about 100 mg twice a week for five weeks (i.e., ten total doses). Alternatively, the compounds of Formula (I) may be administered with a loading dose of about 200 mg followed by about 100 mg doses continuing twice a week. In some embodiments, the administration continues for ten total doses. For instance, the compounds of Formula (I) may be administered at a loading dose of about 200 mg followed by nine additional about 100 mg doses twice a week for a total of ten doses. In one of the embodiments Compounds of Formula (I) can be dosed daily in the range of about 20-200 mg/day or weekly in the range of about 200 mg-2000 mg.

In another embodiment, tablets or suspensions of the compounds of Formula (I) or pharmaceutically acceptable salts thereof, e.g., any one of the Compounds 12-24 or a pharmaceutically acceptable salt thererof, is administered at a dose of about 40-1000 mg daily, once a week (QW) or twice a week (BIW). In another embodiment, tablets or suspensions of the compounds of Formula (I) or pharmaceutically acceptable salts thereof, e.g., any one of the Compounds 12-24 or a pharmaceutically acceptable salt thererof, is administered at a dose of about 40-400 mg daily, once a week (QW) or twice a week (BIW).

In another embodiment, the present disclosure provides compositions (e.g., pharmaceutical compositions) with desirable pharmacokinetic characteristics. For example, the compositions of the invention may provide a blood level of the compounds of Formula (I) or pharmaceutically acceptable salts thereof, e.g., any one of the Compounds 12-24 or a pharmaceutically acceptable salt thererof, which, after metabolism to the therapeutically-active form (e.g., the triphosphate equivalent), results in blood levels of the metabolite that do not induce toxicity (e.g., nephrotoxicity).

An effective amount of a pharmaceutical agent is that which provides an objectively identifiable improvement as noted by the clinician or other qualified observer. As used herein, the term “dosage effective manner” refers to amount of an active compound to produce the desired biological effect in a subject or cell.

In another embodiment, the compounds of Formula (I) or pharmaceutically acceptable salts thereof, e.g., any one of the Compounds 12-24 or a pharmaceutically acceptable salt thererof, is administered to a subject as a single dose. In another embodiment, the compounds of Formula (I) or pharmaceutically acceptable salts thereof, e.g., any one of the Compounds 12-24 or a pharmaceutically acceptable salt thererof, is administered to a subject in multiple doses. Multiple doses can be administered regularly, for example, once every 12 hours, once a day, every 2 days, every 3 days, every 4 days, every 5 days, every 6 days, every 7 days, every 8 days, every 9 days, every 10 days, every 11 days, every 12 days, every 13 days, every 14 days or every 15 days. For example, doses can be administered twice per week. Moreover, each individual dose can be administered with the same or a different dosage.

For example, a subject can be administered any one the compounds of Formula (I) or pharmaceutically acceptable salts thereof, e.g., any one of the Compounds 12-24 or a pharmaceutically acceptable salt thererof, with a first dose of about 1-20 mg/kg (e.g., about 1-1.1 mg/kg, about 1.1-1.2 mg/kg, about 1.2-1.3 mg/kg, about 1.3-1.4 mg/kg, about 1.4-1.5 mg/kg, about 1.5-1.6 mg/kg, about 1.6-1.7 mg/kg, about 1.7-1.8 mg/kg, about 1.8-1.9 mg/kg, about 1.9-2.0 mg/kg, about 2.0-2.1 mg/kg, about 2.1-2.2 mg/kg, about 2.2-2.3 mg/kg, about 2.3-2.4 mg/kg, about 2.4-2.5 mg/kg, about 2.5-2.6 mg/kg, about 2.6-2.7 mg/kg, about 2.7-2.8 mg/kg, about 2.8-2.9 mg/kg, about 2.9-3.0 mg/kg, about 3.0-3.1 mg/kg, about 3.1-3.2 mg/kg, about 3.2-3.3 mg/kg, about 3.3-3.4 mg/kg, about 3.4-3.5 mg/kg, about 3.5-3.6 mg/kg, about 3.6-3.7 mg/kg, about 3.7-3.8 mg/kg, about 3.8-3.9 mg/kg, or about 3.9-4.0 mg/kg) of any one of the Compounds 12-24 (or a pharmaceutically acceptable salt thereof) followed by one or more additional doses at 1-4 mg/kg (e.g., about 1-1.1 mg/kg, about 1.1-1.2 mg/kg, about 1.2-1.3 mg/kg, about 1.3-1.4 mg/kg, about 1.4-1.5 mg/kg, about 1.5-1.6 mg/kg, about 1.6-1.7 mg/kg, about 1.7-1.8 mg/kg, about 1.8-1.9 mg/kg, about 1.9-2.0 mg/kg, about 2.0-2.1 mg/kg, about 2.1-2.2 mg/kg, about 2.2-2.3 mg/kg, about 2.3-2.4 mg/kg, about 2.4-2.5 mg/kg, about 2.5-2.6 mg/kg, about 2.6-2.7 mg/kg, about 2.7-2.8 mg/kg, about 2.8-2.9 mg/kg, about 2.9-3.0 mg/kg, about 3.0-3.1 mg/kg, about 3.1-3.2 mg/kg, about 3.2-3.3 mg/kg, about 3.3-3.4 mg/kg, about 3.4-3.5 mg/kg, about 3.5-3.6 mg/kg, about 3.6-3.7 mg/kg, about 3.7-3.8 mg/kg, about 3.8-3.9 mg/kg, 3.9-4.0 mg/kg, about 4.0-5.0 mg/kg, about 5.0-6.0 mg/kg, about 6.0-7.0 mg/kg, about 7.0-8.0 mg/kg, about 8.0-9.0 mg/kg, about 9.0-10.0 mg/kg, or about 10-20 mg/kg) any one of the Compounds 12-24 (or a pharmaceutically acceptable salt thereof) in the same week or in the following week. For example, a subject can be administered with a first dose of about 3 mg/kg followed by one or more additional doses at about 1 mg/kg. For example, a subject can be administered with a first dose of about 2 mg/kg followed by one or more additional doses at about 3 mg/kg. For example, a subject can be administered with a first dose of 4 mg/kg followed by one or more additional doses at about 4 mg/kg.

Multiple doses can also be administered at variable time intervals. For example, the first 2, 3, 4, 5, 6, 7, or 8 or more doses can be administered at an interval of 6 days followed by additional doses administered at an interval of 7 days. For example, the first 2, 3, 4, 5, 6, 7, or 8 or more doses can be administered at an interval of 7 days followed by additional doses administered at an interval of 3 days.

In one embodiment, the compounds of Formula (I) or pharmaceutically acceptable salts thereof, e.g., any one of the Compounds 12-24 or a pharmaceutically acceptable salt thererof, is administered to a subject once a week at a dose of about 40-1000 mg, or twice a week at a dose of about 40-1000 mg.

In additional embodiments, the present disclosure provides a method of delaying onset, reducing risk, or treating end-organ damage or impairment in a subject infected with BKV, the method comprising orally administering to the subject a pharmaceutical composition comprising a therapeutically effective dose of a compound selected from Compounds 12-24 or pharmaceutically acceptable salts thereof.

In some embodiments the subjects for treatment with one or more compounds of the present disclosure are post-HSCT subjects. In other embodiments, the subjects treated with one or more compounds of the present disclosure have end organ damage, wherein the affected organs includes but are not limited to kidney, ureter, urinary bladder, prostate, or the urethra.

In some embodiments the subjects for treatment with one or more compounds of the present disclosure are HCV subjects. In other embodiments, the subjects treated with one or more compounds of the present disclosure have end organ damage, wherein the affected organs includes but are not limited to kidney, ureter, urinary bladder, prostate, or the urethra.

In some embodiments the present disclosure provides a method of reducing incidence of HCV by orally administering to the subject a pharmaceutical composition including a therapeutically effective dose of a compound selected from Compounds 12-24 or pharmaceutically acceptable salts thereof.

In some embodiments the present disclosure provides a method of reducing incidence of hematuria or renal impairment in a subject at risk of BKV infection reactivation by orally administering to the subject a pharmaceutical composition including a therapeutically effective dose of a compound selected from Compounds 12-24 or pharmaceutically acceptable salts thereof.

In some embodiments the compounds of the present disclosure reduce incidence of hematuria or renal impairment in a subject at risk of BKV infection reactivation, where the subject is a post-HSCT subject. In additional embodiments, the present disclosure provides pharmaceutical compositions of the compounds of Formula (I) or pharmaceutically acceptable salts thereof for reducing BKV infection reactivation in said subject. The pharmaceutical compositions of the present disclosure lowers BK viral load in a subject, and delays onset of or reduces risk of end-organ damage or impairment. The end organs include but are not limited tokidney, ureter, urinary bladder, prostate, and urethra.

In some embodiments the pharmaceutical composition of the present disclosure is administered daily, once a week (QW), or twice a week (BIW) with about 40-1000 mg of compounds of Formula (I), e.g., Compounds 12-24, or pharmaceutically acceptable salts thereof. The pharmaceutical compositions of the present disclosure is administered daily, once a week (QW), or twice a week (BIW) with about 40 mg, 50 mg, 75 mg, 100 mg, 150 mg, 175 mg, 200 mg, 250 mg, 275 mg, 300 mg, 325 mg, 350 mg, 375 mg, 400 mg, 450 mg, 500 mg, 500-600 mg, 600-700 mg, 700-800 mg, 800-900 mg, or 900-1000 mg, or twice a week (BIW) with about 40 mg, 50 mg, 75 mg, 100 mg, 150 mg, 175 mg, 200 mg, 250 mg, 275 mg, 300 mg, 325 mg, 350 mg, 375 mg, or 400 mg, 450 mg, 500 mg, 500-600 mg, 600-700 mg, 700-800 mg, 800-900 mg, or 900-1000 mg of Compounds 12-24 or pharmaceutically acceptable salts thereof.

Compounds of the current disclosure are administered at dose of about 1-20 mg/kg, for example, 1.25 mg/kg, 2.5 mg/kg, 5.0 mg/kg, 10 mg/kg, or 20 mg/kg on day 1, 2, 3, 4, 5, 6, 7, or up 10 days after post-HSCT. The 1-20 mg/kg of the compounds of the present disclosure may be administered once a week or twice a week. In one embodiment, the treatment is initiated with once a week administration of 1-20 mg/kg and then followed by bi-weekly administration of 1-20 mg/kg until necessary.

The present disclosure provides compounds of Formula (I), e.g., any one of Compounds 12-24 (or pharmaceutically acceptable salts thereof) administered at a dose of about 1-20 mg/kg (e.g., about 1-1.1 mg/kg, about 1.1-1.2 mg/kg, about 1.2-1.3 mg/kg, about 1.3-1.4 mg/kg, about 1.4-1.5 mg/kg, about 1.5-1.6 mg/kg, about 1.6-1.7 mg/kg, about 1.7-1.8 mg/kg, about 1.8-1.9 mg/kg, about 1.9-2.0 mg/kg, about 2.0-2.1 mg/kg, about 2.1-2.2 mg/kg, about 2.2-2.3 mg/kg, about 2.3-2.4 mg/kg, about 2.4-2.5 mg/kg, about 2.5-2.6 mg/kg, about 2.6-2.7 mg/kg, about 2.7-2.8 mg/kg, about 2.8-2.9 mg/kg, about 2.9-3.0 mg/kg, about 3.0-3.1 mg/kg, about 3.1-3.2 mg/kg, about 3.2-3.3 mg/kg, about 3.3-3.4 mg/kg, about 3.4-3.5 mg/kg, about 3.5-3.6 mg/kg, about 3.6-3.7 mg/kg, about 3.7-3.8 mg/kg, about 3.8-3.9 mg/kg, about 3.9-4.0 mg/kg, about 4.0-5.0 mg/kg, about 5.0-6.0 mg/kg, about 6.0-7.0 mg/kg, about 7.0-8.0 mg/kg, about 8.0-9.0 mg/kg, about 9.0-10 mg/kg, about 10-15 mg/kg, or about 15-20 mg/kg).

In some embodiments the present disclosure provides compounds of Formula (I), e.g., any one of Compounds 12-24 (or pharmaceutically acceptable salts thereof) formulated as a pharmaceutical composition. In one embodiment, compounds of Formula (I), e.g., any one of Compounds 12-24 (or pharmaceutically acceptable salts thereof) is formulated as a tablet. In another embodiment, compounds of Formula (I), e.g., any one of Compounds 12-24 (or pharmaceutically acceptable salts thereof) is formulated as a suspension.

The present disclosure provides treatment and/or prevention of a viral infection with the compounds of the invention. The compounds represented by Formula (I) are used in treating, preventing, and/or manufacturing a medicament for treating and/or preventing at least one virus selected from adenovirus, CMV, JCV, BKV, SV40, MCV, KIV, WUV, EBV, vaccinia, herpes simplex virus 1 (HSV-1), herpes simplex virus 2 (HSV-2), human herpes virus 6 (HHV-6), human herpes virus 8 (HHV-8), hepatitis B virus, hepatitis C virus, varicella zoster virus (VZV), variola major, variola minor, smallpox, cowpox, camelpox, monkeypox, poliovirus, ebola virus, Marburg virus, enterovirus (e.g., EV68 and EV71), papilloma virus, human immunodeficiency virus (HIV), influenza, and any combination thereof.

In some embodiments the present disclosure provides a method of treatment, prevention, or delaying on-set of CMV infection or a CMV infection associated disease or disorder, by oral administration to a subject in need thereof a pharmaceutical composition of a therapeutically effective dose of a compound selected from Compounds 12-24, or a pharmaceutically acceptable salt thereof.

In some embodiments the present invention provides a method of treatment, prevention, or delaying on-set of HCV infection or a HCV infection associated disease or disorder, by oral administration to a subject in need thereof a pharmaceutical composition of a therapeutically effective dose of a compound selected from Compounds 12-24, or a pharmaceutically acceptable salt thereof.

In some embodiments the present invention provides a method of treatment, prevention, or delaying on-set of Marburg virus infection or Marburg virus infection associated disease or disorder, by oral administration to a subject in need thereof a pharmaceutical composition of a therapeutically effective dose of a compound selected from Compounds 12-24, or a pharmaceutically acceptable salt thereof.

In some embodiments the present invention provides a method of treatment, prevention, or delaying on-set of Ebola virus infection or Ebola virus infection associated disease or disorder, by oral administration to a subject in need thereof a pharmaceutical composition of a therapeutically effective dose of a compound selected from Compounds 12-24, or a pharmaceutically acceptable salt thereof.

In some embodiments the present disclosure provides a method of treatment, prevention, or delaying on-set of enterovirus infection or enterovirus infection associated disease or disorder, by oral administration to a subject in need thereof a pharmaceutical composition of a therapeutically effective dose of a compound selected from Compounds 12-24, or a pharmaceutically acceptable salt thereof.

The subject treated for a viral infection (e.g., a CMV infection or a CMV infection associated disease or disorder or a HCV infection or a HCV infection associated disease or disorder) is administered once or twice a week with about 40 mg, 50 mg, 75 mg, 100 mg, 150 mg, 175 mg, 200 mg, or 250 mg of a compound selected from Compounds 12-24, or a pharmaceutically acceptable salt thereof. The present disclosure provides treatment of a subject for CMV infection or a CMV infection associated disease or disorder or a HCV infection or a HCV infection associated disease or disorder by administering to the subject once a week (QW) about 200 mg or twice a week (BIW) about 100 mg of a compound selected from Compounds 12-24, or a pharmaceutically acceptable salt thereof. In one embodiment, the subject is treated twice a week (BIW) with about 100 mg of the compound. In another embodiment, the subject is treated once a week (QW) with about 200 mg, or twice a week (BIW) with about 100 mg of the compound. The subject treated for CMV infection or a CMV infection associated disease or disorder or a HCV infection or a HCV infection associated disease or disorder is a HSCT subject and receives an allogeneic stem cell transplant.

The present disclosure also provides a method of prophylactic treatment, prevention, or delaying on-set of CMV infection or a CMV infection associated disease or disorder, by orally administering to a subject a pharmaceutical composition comprising a therapeutically effective dose of a compound selected from Compounds 12-24, or a pharmaceutically acceptable salt thereof.

The present disclosure also provides a method of prophylactic treatment, prevention, or delaying on-set of HCV infection or a HCV infection associated disease or disorder, by orally administering to a subject a pharmaceutical composition comprising a therapeutically effective dose of a compound selected from Compounds 12-24, or a pharmaceutically acceptable salt thereof.

The present disclosure further provides a method of prophylactic treatment, prevention, or delaying on-set of Marburg virus infection or a Marburg infection associated disease or disorder, by orally administering to a subject a pharmaceutical composition comprising a therapeutically effective dose of a compound selected from Compounds 12-24 or a pharmaceutically acceptable salt thereof, in combination with one or more of compound or composition selected from an immunosuppressant and an antiviral agent.

The present disclosure further provides a method of prophylactic treatment, prevention, or delaying on-set of Ebola infection or a Ebola infection associated disease or disorder, by orally administering to a subject a pharmaceutical composition comprising a therapeutically effective dose of a compound selected from Compounds 12-24 or a pharmaceutically acceptable salt thereof, in combination with one or more of compound or composition selected from an immunosuppressant and an antiviral agent.

The present disclosure further provides a method of prophylactic treatment, prevention, or delaying on-set of enterovirus infection or an enterovirus infection associated disease or disorder, by orally administering to a subject a pharmaceutical composition comprising a therapeutically effective dose of a compound selected from Compounds 12-24 or a pharmaceutically acceptable salt thereof, in combination with one or more of compound or composition selected from an immunosuppressant and an antiviral agent.

The present disclosure further provides a method of treatment, prevention, or delaying on-set of viral infections or viral-infection-associated diseases or disorders (e.g., human cytomegalovirus (HCMV), BK virus (BKV), Epstein-Barr virus (EBV), adenovirus, JC virus (JCV), SV40, MC virus (MCV), KI virus (KIV), WU virus (WUV), vaccinia, herpes simplex virus 1 (HSV-1), herpes simplex virus 2 (HSV-2), human herpes virus 6 (HHV-6), human herpes virus 8 (HHV-8), hepatitis B virus, hepatitis C virus, varicella zoster virus (VZV), variola major, variola minor, smallpox, cowpox, camelpox, monkeypox, poliovirus, ebola virus, Marburg virus, enterovirus, papilloma virus, and human immunodeficiency virus (HIV)) by orally administering to a subject a pharmaceutical composition comprising a therapeutically effective dose of a compound selected from Compounds 12-24 or a pharmaceutically acceptable salt thereof, in combination with one or more of compound or composition selected from an immunosuppressant and an antiviral agent.

In some embodiments, the pharmaceutical composition of the present disclosure is administered in combination with one or more compounds or compositions selected from midazolam, cyclosporine A, tacrolimus, ganciclovir, valganciclovir, foscavir, cidofovir, second-line anti-CMV drugs, second-line anti-HCV drugs, foscarnet, filgrastim, pegfilgrastim, corticosteroids such as budesonide, beclomethasone, and broad-spectrum CYP inhibitor aminobenzotriazole or combinations thereof.

In some embodiments, the present disclosure also relates to treatment of a PV-associated, e.g., JCV-associated, multifocal leukoencephalopathy (PVML) or PV-associated nephropathy with one of the disclosed compounds, e.g., Compounds 12-24, or pharmaceutically acceptable salts thereof.

The present disclosure provides treating subjects with compounds of Formula (I) or pharmaceutically acceptable salt thereof, wherein the subject is immunocompromised. In one embodiment, the immunocompromised subject is a transplant patient on immunosuppressive medications. In some embodiments, the immunocompromised subject is infected with HIV.

In additional embodiments, the compound is for administration in combination with at least one other immunosuppressant agent. In one embodiment, the immunosuppressant agent is concurrently or sequentially administered. The immunosuppressant agents include but are not limited to Daclizumab, Basiliximab, Tacrolimus, Sirolimus, Mycophenolate, Cyclosporine A, Glucocorticoids, Anti-CD3 monoclonal antibodies, Antithymocyte globulin, Anti-CD52 monoclonal antibodies, Azathioprine, Everolimus, Dactinomycin, Cyclophosphamide, Platinum, Nitrosurea, Methotrexate, Mercaptopurine, Muromonab, IFN gamma, Infliximab, Etanercept, Adalimumab, Natalizumab, Fingolimod, and combinations thereof.

In another embodiment, the invention provides an oral dosage form comprising the compounds of Formula (I) or pharmaceutically acceptable salts thereof, e.g., any one of the Compounds 12-24 or a pharmaceutically acceptable salt thererof, having a purity of equal to or greater than about 91% for the therapeutic and/or prophylactic treatment of viral infection in a subject, wherein said oral dosage form, upon administration to a human at a dosage of about 1-20 mg/kg (e.g., about 1-1.1 mg/kg, about 1.1-1.2 mg/kg, about 1.2-1.3 mg/kg, about 1.3-1.4 mg/kg, about 1.4-1.5 mg/kg, about 1.5-1.6 mg/kg, about 1.6-1.7 mg/kg, about 1.7-1.8 mg/kg, about 1.8-1.9 mg/kg, about 1.9-2.0 mg/kg, about 2.0-2.1 mg/kg, about 2.1-2.2 mg/kg, about 2.2-2.3 mg/kg, about 2.3-2.4 mg/kg, about 2.4-2.5 mg/kg, about 2.5-2.6 mg/kg, about 2.6-2.7 mg/kg, about 2.7-2.8 mg/kg, about 2.8-2.9 mg/kg, about 2.9-3.0 mg/kg, about 3.0-3.1 mg/kg, about 3.1-3.2 mg/kg, about 3.2-3.3 mg/kg, about 3.3-3.4 mg/kg, about 3.4-3.5 mg/kg, about 3.5-3.6 mg/kg, about 3.6-3.7 mg/kg, about 3.7-3.8 mg/kg, about 3.8-3.9 mg/kg, 3.9-4.0 mg/kg, about 4.0-5.0 mg/kg, about 5.0-6.0 mg/kg, about 6.0-7.0 mg/kg, about 7.0-8.0 mg/kg, about 8.0-9.0 mg/kg, about 9.0-10.0 mg/kg, or about 10-20 mg/kg) of said compound, provides an EC₅₀ (μM) against HCMV UL54 resistant mutant AD169 of between about 0.06-0.04, for example, about 0.059, 0.058, 0.057, 0.056, 0.055, 0.054, 0.053, 0.052, 0.051, 0.050, 0.049, 0.048, 0047, 0.046, 0.045, 0.044, 0.043, 0.042, 0.041, or 0.040. In one embodiment EC₅₀ (μM) of Compound 12 and/or Compound 13 against HCMV UL54 resistant mutant AD169 is about 0.052. EC₅₀ is determined by any of the well-known methods in the art and as described in the examples herein.

In another embodiment, the invention provides an oral dosage form comprising the compounds of Formula (I) or pharmaceutically acceptable salts thereof, e.g., any one of the Compounds 12-24 or a pharmaceutically acceptable salt thererof, having a purity of equal to or greater than about 91% for the therapeutic and/or prophylactic treatment of viral infection in a subject, wherein said oral dosage form, upon administration to a human at a dosage of about 1-20 mg/kg (e.g., about 1-1.1 mg/kg, about 1.1-1.2 mg/kg, about 1.2-1.3 mg/kg, about 1.3-1.4 mg/kg, about 1.4-1.5 mg/kg, about 1.5-1.6 mg/kg, about 1.6-1.7 mg/kg, about 1.7-1.8 mg/kg, about 1.8-1.9 mg/kg, about 1.9-2.0 mg/kg, about 2.0-2.1 mg/kg, about 2.1-2.2 mg/kg, about 2.2-2.3 mg/kg, about 2.3-2.4 mg/kg, about 2.4-2.5 mg/kg, about 2.5-2.6 mg/kg, about 2.6-2.7 mg/kg, about 2.7-2.8 mg/kg, about 2.8-2.9 mg/kg, about 2.9-3.0 mg/kg, about 3.0-3.1 mg/kg, about 3.1-3.2 mg/kg, about 3.2-3.3 mg/kg, about 3.3-3.4 mg/kg, about 3.4-3.5 mg/kg, about 3.5-3.6 mg/kg, about 3.6-3.7 mg/kg, about 3.7-3.8 mg/kg, about 3.8-3.9 mg/kg, 3.9-4.0 mg/kg, about 4.0-5.0 mg/kg, about 5.0-6.0 mg/kg, about 6.0-7.0 mg/kg, about 7.0-8.0 mg/kg, about 8.0-9.0 mg/kg, about 9.0-10.0 mg/kg, or about 10-20 mg/kg) of said compound, provides an EC₅₀ μM against HCMV UL54 resistant mutant D542E of between about 6.0-4.0, for example, 6.0-5.9, 5.9-5.8, 5.8-5.7, 5.7-5.6, 5.6-5.5, 5.5-5.4, 5.4-5.3, 5.3-5.2, 5.2-5.1, 5.1-5.0, 5.0-4.9, 4.9-4.8, 4.8-4.7, 4.7 4.6, 4.6-4.5, 4.5-4.4, 4.4-4.3, 4.3-4.2, 4.2-4.1, or 4.1-4.0. In one embodiment, EC₅₀ (μM) of Compound 12 and/or Compound 13 against HCMV UL54 resistant mutant D542E is about 5.41.

In another embodiment, the invention provides an oral dosage form comprising the compounds of Formula (I) or pharmaceutically acceptable salts thereof, e.g., any one of the Compounds 12-24 or a pharmaceutically acceptable salt thererof, having a purity of equal to or greater than about 91% for the therapeutic and/or prophylactic treatment of viral infection in a subject, wherein said oral dosage form, upon administration to a human at a dosage of about 1-20 mg/kg (e.g., about 1-1.1 mg/kg, about 1.1-1.2 mg/kg, about 1.2-1.3 mg/kg, about 1.3-1.4 mg/kg, about 1.4-1.5 mg/kg, about 1.5-1.6 mg/kg, about 1.6-1.7 mg/kg, about 1.7-1.8 mg/kg, about 1.8-1.9 mg/kg, about 1.9-2.0 mg/kg, about 2.0-2.1 mg/kg, about 2.1-2.2 mg/kg, about 2.2-2.3 mg/kg, about 2.3-2.4 mg/kg, about 2.4-2.5 mg/kg, about 2.5-2.6 mg/kg, about 2.6-2.7 mg/kg, about 2.7-2.8 mg/kg, about 2.8-2.9 mg/kg, about 2.9-3.0 mg/kg, about 3.0-3.1 mg/kg, about 3.1-3.2 mg/kg, about 3.2-3.3 mg/kg, about 3.3-3.4 mg/kg, about 3.4-3.5 mg/kg, about 3.5-3.6 mg/kg, about 3.6-3.7 mg/kg, about 3.7-3.8 mg/kg, about 3.8-3.9 mg/kg, 3.9-4.0 mg/kg, about 4.0-5.0 mg/kg, about 5.0-6.0 mg/kg, about 6.0-7.0 mg/kg, about 7.0-8.0 mg/kg, about 8.0-9.0 mg/kg, about 9.0-10.0 mg/kg, or about 10-20 mg/kg) of said compound, provides an EC₅₀ (μM) against HCMV UL54 resistant mutant GDF^{R}P53 of about 0.2-0.15, for example, 0.2-0.19, 0.19-0.18, 0.18-0.17, 0.17-0.16, or 0.16-0.15. In one embodiment, EC₅₀ (μM) of Compound 12 and/or Compound 13 against HCMV UL54 resistant mutant GDF^{R}P53 is about 0.171.

In another embodiment, the invention provides an oral dosage form comprising the compounds of Formula (I) or pharmaceutically acceptable salts thereof, e.g., any one of the Compounds 12-24 or a pharmaceutically acceptable salt thererof, having a purity of equal to or greater than about 91% for the therapeutic and/or prophylactic treatment of viral infection in a subject, wherein said oral dosage form, upon administration to a human at a dosage of about 1-20 mg/kg (e.g., about 1-1.1 mg/kg, about 1.1-1.2 mg/kg, about 1.2-1.3 mg/kg, about 1.3-1.4 mg/kg, about 1.4-1.5 mg/kg, about 1.5-1.6 mg/kg, about 1.6-1.7 mg/kg, about 1.7-1.8 mg/kg, about 1.8-1.9 mg/kg, about 1.9-2.0 mg/kg, about 2.0-2.1 mg/kg, about 2.1-2.2 mg/kg, about 2.2-2.3 mg/kg, about 2.3-2.4 mg/kg, about 2.4-2.5 mg/kg, about 2.5-2.6 mg/kg, about 2.6-2.7 mg/kg, about 2.7-2.8 mg/kg, about 2.8-2.9 mg/kg, about 2.9-3.0 mg/kg, about 3.0-3.1 mg/kg, about 3.1-3.2 mg/kg, about 3.2-3.3 mg/kg, about 3.3-3.4 mg/kg, about 3.4-3.5 mg/kg, about 3.5-3.6 mg/kg, about 3.6-3.7 mg/kg, about 3.7-3.8 mg/kg, about 3.8-3.9 mg/kg, 3.9-4.0 mg/kg, about 4.0-5.0 mg/kg, about 5.0-6.0 mg/kg, about 6.0-7.0 mg/kg, about 7.0-8.0 mg/kg, about 8.0-9.0 mg/kg, about 9.0-10.0 mg/kg, or about 10-20 mg/kg) of said compound, provides an EC₅₀ (μM) against HCMV UL54 resistant mutant 4955^{R} of between 0.2-0.11, for example, 0.2-0.19, 0.19-0.18, 0.18-0.17, 0.17-0.16, 0.16-0.15, 0.15-0.14, 0.14-0.13, 0.13-0.12, or 0.12-0.11. In one embodiment, EC₅₀ (μM) of Compound 12 and/or Copmpound 13 against HCMV UL54 resistant mutant 4955^{R} is about 0.143.

In some embodiments, the compounds of the present invention have activity against against various viruses (e.g., herpes simplex virus and HCMV). Assays for biological activity can be carried out in a number of systems, for instance, human foreskin fibroblast cells or Madin-Darby Canine Kidney (MDCK) cells. Some examples of biological activity of the compounds of the present invention are given below.

TABLE 2
Activity of Compound 12 against Herpes Simplex Virus 1 (Strain E-
377) and HCMV (Strain AD169) in human foreskin fibroblast cells

| | HSV-1 | HSV-1 | | HCMV | HCMV | |
|---|---|---|---|---|---|---|
| | EC₅₀, | CC₅₀, | HSV-1 | EC₅₀, | CC₅₀, | HCMV |
| Compound | μM | μM | SI₅₀ | μM | μM | SI₅₀ |
| Compound 12 | >50 | >50 | 1 | 0.052 | >50 | >961 |
| Acyclovir | 2.2 | >100 | >45 | | | |
| Ganciclovir | | | | 1.5 | >100 | >67 |

TABLE 3
Activity of Compound 12 and Hexadecyloxypropyl-Cidofovir
(HDP-CDV) against HCMV UL54 resistant mutants in human
foreskin fibroblast cells

| | Compound | Compound | HDP-CDV | HDP-CDV |
|---|---|---|---|---|
| | 12 EC₅₀ | 12 EC₉₀ | EC₅₀ | EC₉₀ |
| Virus | μM | μM | μM | μM |
| AD169 | 0.052 | 0.15 | 0.001 | |
| D542E | 5.41 | >10 | 0.0295 | 0.1529 |
| (HDP-CDV^{R}) | | | | |
| GDG^{R}P53 | 0.171 | 1.552 | 0.0053 | 0.0316 |
| 4955^{R} | 0.143 | 0.372 | 0.0013 | 0.006 |

TABLE 4
Activity of Compound 12 against Vaccinia Virus
(Strain Copenhagen) and Cowpox Virus (Strain
Brighton) in Human Foreskin Fibroblast Cells

| | VACV | VACV | | CPXV | CPXV | |
|---|---|---|---|---|---|---|
| | EC₅₀, | CC₅₀, | VACV | EC₅₀, | CC₅₀, | CPXV |
| Compound | μM | μM | SI₅₀ | μM | μM | SI₅₀ |
| Compound | >10 | >10 | 1 | >10 | >10 | 1 |
| 12-001 | | | | | | |
| Cidofovir | 22.42 | >300 | >13 | 30.52 | >300 | >10 |

TABLE 5
Activity of Compound 12 against Strains of Influenza virus in
Madin-Darby Canine Kidney (MDCK) cells

| | A/CA/10/2009 | B/FL/4/2006 | Cytotoxicity |
|---|---|---|---|
| Compound | (EC₅₀, μM) | (EC₅₀, μM) | (CC₅₀, μM) |
| Compound 12 | >4 | >4 | 17.18 |
| Oseltamivir | 0.17 | 16.25 | >20 |

TABLE 6
Activity of Compound 12 against Epstein-Barr virus in Akata cells

| | Efficacy | Cytotoxicity |
|---|---|---|
| | DNA Hybridization Assay | CellTiter-GloAssay |
| Compound | (EC₅₀, μM) | (CC₅₀, μM) |
| Compound 12 | >10 | 18 |
| Acyclovir | 7.0 | >100 |

TABLE 7
Activity of Compound 12 and HDP-CDV against HCMV in
MRC-5 cells and BKV in Vero cells

| | CMV | BKV | MT4 | CMV - SI | BKV - SI |
|---|---|---|---|---|---|
| | EC₅₀ | EC₅₀ | CC₅₀ | (MT4 CC₅₀/ | (MT4 CC₅₀/ |
| Compound | (μM) | (μM) | (μM) | EC₅₀) | EC₅₀) |
| Compound 12 | 0.027 | 1.55 | 25.6 | 948 | 16.5 |
| | (n = 4) | (n = 2) | (n = 7) | | |
| HDP-CDV | 0.0004 | 0.018 | 0.060 | 150 | 3.3 |
| | (n = 8) | (n = 11) | (n = 6) | | |

TABLE 8
In Vitro Antiviral Activity of Compound 12

| Virus | EC₅₀ (μM) | CC₅₀ (μM) | SI | Cell line |
|---|---|---|---|---|
| HCMV | 0.017, n = 12 | 50, n = 4 | 2941 | MRC5 |
| MCMV | 1.6, n = 2 | 60, n = 1 | 37.5 | MEF |
| HHV-8 | <0.08, n = 1 | >10, n = 1 | >125 | BCBL-1 |
| HCV | 0.57, n = 1 | >20, n = 1 | >35 | Huh |
| BKV | 2.1, n = 4 | 93, n = 1 | 44 | Vero |
| HBV | 3, n = 1 | 34, n = 1 | 11 | 2.2.15 |
| Poliovirus 3 | 3.3, n = 1 | 29, n = 1 | 9 | Vero 76 |
| HHV-6B | 4.7, n = 1 | 8.4, n = 1 | ~2 | MOLT-3 |
| Ebola | 1.0, n = 1 | 36.9, n = 1 | 37 | Huh |

TABLE 9
In Vitro Activity Comparison (Mean XC₅₀)

| | Compound | | |
|---|---|---|---|
| | Brincidofovir (BCV) | Compound 12 | Compound 24 |
| HCMV EC₅₀ | 0.0003 | 0.017 | 0.008 |
| (uM) | N = 9 | N = 10 | N = 4 |
| MT4 | 0.06 | 24.7 | 13.5 |
| cytotoxicity | N = 10 | N = 8 | N = 6 |
| CC₅₀ (uM) | | | |

The present disclosure provides compounds of Formula (I) or pharmaceutically accepatable salts thereof, e.g., any one of the compounds of Compounds 12-24 or a pharmaceutically acceptable salt thereof, with an improved efficacy/toxicity ratio compared to HDP-CDV. In some embodiments, the compounds disclosed herein have a Selective Index (SI) value of between 900-1000 for CMV and/or SI value between 15-20 for BKV. In one embodiment, Compound 12 and/or Compound 13 has a SI (Selective Index) value of about 948 for CMV and about 16.5 for BKV, while HDP-CDV has a SI value of about 150 for CMV and about 3.3 for BKV.

In another embodiment, the invention provides an oral dosage form comprising any one of the Compounds 12-24 (or a pharmaceutically acceptable salt thereof) having a purity of equal to or greater than about 91% for the therapeutic and/or prophylactic treatment of viral infection in a subject, wherein said oral dosage form, upon administration to a human at a dosage of about 1-20 mg/kg (e.g., about 1-1.1 mg/kg, about 1.1-1.2 mg/kg, about 1.2-1.3 mg/kg, about 1.3-1.4 mg/kg, about 1.4-1.5 mg/kg, about 1.5-1.6 mg/kg, about 1.6-1.7 mg/kg, about 1.7-1.8 mg/kg, about 1.8-1.9 mg/kg, about 1.9-2.0 mg/kg, about 2.0-2.1 mg/kg, about 2.1-2.2 mg/kg, about 2.2-2.3 mg/kg, about 2.3-2.4 mg/kg, about 2.4-2.5 mg/kg, about 2.5-2.6 mg/kg, about 2.6-2.7 mg/kg, about 2.7-2.8 mg/kg, about 2.8-2.9 mg/kg, about 2.9-3.0 mg/kg, about 3.0-3.1 mg/kg, about 3.1-3.2 mg/kg, about 3.2-3.3 mg/kg, about 3.3-3.4 mg/kg, about 3.4-3.5 mg/kg, about 3.5-3.6 mg/kg, about 3.6-3.7 mg/kg, about 3.7-3.8 mg/kg, about 3.8-3.9 mg/kg, 3.9-4.0 mg/kg, about 4.0-5.0 mg/kg, about 5.0-6.0 mg/kg, about 6.0-7.0 mg/kg, about 7.0-8.0 mg/kg, about 8.0-9.0 mg/kg, about 9.0-10.0 mg/kg, or about 10-20 mg/kg) of any one of the Compounds 12-24 (or a pharmaceutically acceptable salt thereof) and metabolism of said compound of any one of the Compounds 12-24 (or a pharmaceutically acceptable salt thereof) to the triphosphate equivalent, provides a Cₘₐₓ of said triphosphate equivalent that is less than about 30% of the Cₘₐₓ of said compound any one of the Compounds 12-24 (or a pharmaceutically acceptable salt thereof), e.g., less that about 20% of the Cₘₐₓ of said compound. In some embodiments, the Cₘₐₓ of the metabolite (i.e., triphosphate equivalent) is less than about 50%, 45%, 40%, 35%, 30%, 25%, 20%, 15%, or 10% of the Cₘₐₓ of any one of the Compounds 12-24 (or a pharmaceutically acceptable salt thereof).

The pharmacokinetic behavior of a composition will vary somewhat from subject to subject within a population. The numbers described above for the compositions of the invention are based on the average behavior in a population. The present invention is intended to encompass compositions that on average fall within the disclosed ranges, even though it is understood that certain subjects may fall outside of the ranges.

The pharmaceutical compositions can be included in a container, pack, or dispenser together with instructions for administration. The present disclosure provides a kit including, in addition to a pharmaceutical composition of any one of the disclosed compounds, a container, pack, or dispenser together with instructions for administration.

The compounds of the present invention are capable of further forming salts. All of these forms are also contemplated within the scope of the claimed invention.

### Methods for Preventing Disease or Disorder Due to Virus Reactivation

The current invention also provides a method of preventing a disease or disorder in a subject at risk of virus infection reactivation, by orally administering to the subject a pharmaceutical composition of a therapeutically effective dose of a compound of Formula (I) or a pharmaceutically acceptable salt thereof, e.g., any one of the Compounds 12-24 or a pharmaceutically acceptable salt thereof. In some embodiments, the virus at risk of reactivation can be BKV. In some preferred embodiments, the virus at risk of reactivation can be CMV.

In one embodiment, the subject at risk of virus infection reactivation may be stem cell transplant or renal transplant recipients. In an embodiment, the subject may be a post-HSCT subject. In yet other embodiments, the subject may be islet cell transplant recipient, bone marrow transplant recipient, endothelial cell transplant recipient, epidermal cell transplant recipient, myoblast transplant recipient, muscle derived stem cell recipient, and/or neural stem cell transplant recipient.

In yet other embodiments, the subject may be islet cell transplant recipient, bone marrow transplant recipient, endothelial cell transplant recipient, epidermal cell transplant recipient, myoblast transplant recipient and/or neural stem cell transplant recipient.

In yet another embodiment, the method of the current invention prevents hematuria or renal impairment in a post-HSCT subject. The prevention of hematuria or renal impairment in post-HSCT patient may be associated with prevention of viral reactivation in the subject. In one embodiment, the prevention of virus infection reactivation prevents hematuria or renal impairment in said subject.

Method for Reducing the Incidence of BKV Associate Hematuria and/or Renal Impairment

The present application also relates to methods for reducing the incidence of BKV associated hematuria and/or renal impairment. The methods of the current invention prevent the emergence of hematuria and renal impairment, both associated with end-organ damages from BKV infection. The invention also relates to a method of reducing risk of and/or delaying onset of BK viral load increase in post-HSCT patients with a compound of Formula (I) or a pharmaceutically acceptable salt thereof, e.g., Compound 12 or Compound 13, or a pharmaceutically acceptable salt thereof, thereby reducing risk of and/or delaying onset of end organ disease in these patients. The pharmaceutical composition of the current invention may prevent end-organ damage or impairment, for example, kidney, ureter, urinary bladder, prostate, and urethra damage or impairment.

In some embodiments, the methods for reducing the incidence of BKV associated hematuria and/or renal impairment provides that about 40-1000 mg of the compound(s) of the current invention is administered once a week (QW) or twice a week (BIW) to a subject for prevention or treatment of end-organ damage or impairment. In one embodiment, the subject is treated QW or BIW with about 40-1000 mg or about 100-200 mg once or twice a week. A subject infected with a dsDNA virus, e.g., BKV, is treated daily, once a week (QW) with about 40-1000 mg or twice a week (BIW) with about 40-1000 mg of a compound of the present disclosure, e.g., Compound 12 or Compound 13, or a pharmaceutically acceptable salt thereof. In further embodiments, the subject is treated with daily, once a week (QW) with about 150 mg or about 200 mg, or twice a week (BIW) with about 75 mg or about 100 mg of a compound of a compound of the present disclosure, e.g., Compound 12 or Compound 13, or a pharmaceutically acceptable salt thereof.

In yet other embodiments, the methods for reducing the incidence of BKV-associated hematuria and/or renal impairment provide that a subject is treated with about 50-99 mg, 101-149 mg, 151-199 mg, 201-250 mg, or >251 mg dose without resulting in significant adverse effects (AEs). In some embodiments, the dose varies within one week, two weeks, or during the entire treatment period.

The impact of the compounds of Formula (I) or pharmaceutically acceptable salts thereof on hemorragic cystitis emergence is assessed based on the incidence of treatment emergent hematuria. The present disclosure provides preventing Hematuria (Hem+) in HSCT patients who are BKV positive at base line (BKU+) with Compound 12 or Compound 13 (or a pharmaceutically acceptable salt thereof). In one embodiment, there is not be a significant difference in Hem+ in patients who are BKV viruria negative (BKU−) at baseline compared to the placebo group.

The impact of the compounds of Formula (I) or pharmaceutically acceptable salts thereof on renal dysfunction in subjects with preexisting BKV infection is measured. For example, the present disclosure provides prevention of an increase in creatinine level and worsening of renal function in patients who were BKV viruria (BKU+) at baseline (post-HSCT engraft) and treated with Compound 12 or Compound 13 (or a pharmaceutically acceptable salt thereof) compared to the placebo group. In one embodiment, Compound 12 or Compound 13 (or a pharmaceutically acceptable salt thereof) does not impact the end organ damage in patients who were BKV viruria negative (BKU−) at baseline. In these patients, the creatinine level does not increase compared to the placebo group.

The present disclosure provides methods for use of compounds of Formula (I) or pharmaceutically acceptable salts thereof, for reducing microscopic hematuria in subjects shedding BKV in their urine. For example, subjects who has BKV viruria during treatment period, i.e., receiving a pharmaceutical composition of Compound 12 or Compound 13, or a pharmaceutically acceptable salt thereof, has a 2-10 fold decrease in blood positive urinalysis compared to subjects receiving placebo. In some embodiments, between the treatment and placebo groups, the difference in blood positive urinalysis may be 2-8, 2-7, 2-6, 2-5, or 2-4 fold. Among subjects without BK viruria, the rates of blood positive urinalysis may be low or comparable between the treated versus the untreated, e.g., patients receiving placebo.

The methods of treatment with compounds of Formula (I), or pharmaceutically acceptable salts thereof, have beneficial effects on BK associated bladder events. For example, high BK viruria measurements (e.g., ≧1×10¹⁰ copies/mL) are associated with clinically important events (e.g., AEs for cystitis or blood in urine). Compared to placebo-treated subjects, the rates of confirmed blood positive urinalyses may occur at 1/10^{th}, 1/9^{th}, 1/8^{th}, 1/7^{th}, 1/6^{th}, 1/5^{th}, 1/4^{th} the rate in Compound 12 or Compound 13 (or a pharmaceutically acceptable salt thereof) treated subjects. In some embodiments, the incidence of sustained BK viruria may be reduced for Compound 12 or Compound 13 (or a pharmaceutically acceptable salt thereof) treated subjects who develop BK viruria during treatment.

The methods of the current embodiments involve measuring serum creatinine concentrations as a marker of renal function. The current methods measure kidney function by calculating creatinine clearance from the body by the kidneys. This is referred to as creatinine clearance and it estimates the rate of filtration by kidneys (glomerular filtration rate, or GFR). The creatinine clearance is measured in two ways. It is calculated by a formula using serum (blood) creatinine level, patient's weight, and age. Creatinine clearance is also measured by collecting a 24-hour urine sample. Normal level of creatinine in blood is 0.7 to 1.3 mg/dL for men and 0.6 to 1.1 mg/dL for women. See Creatinine—Blood, Medline Plus, U.S. National Library of Medicine, NIH. If kidney function is abnormal, creatinine levels will increase in the blood (because less creatinine is released through your urine).

Creatinine level more than about 1.36 mg/mL in urine is considered elevated. In the methods of the current invention, about 15% or about 25% increase in creatinine level from baseline is considered clinically important change during the treatment period.

The current method provides evaluation of microscopic hematuria using heme+1 urinalysis as a surrogate. End of treatment (last value) elevations in serum creatinine measurements (e.g., >120 μM (1.36 mg/dl)) is considered clinically meaningful. Pre-existing renal dysfunction is distinguished by measuring both the last value for creatinine, which is higher than the normal level, e.g., >120 μM and at least 15% or 25% increase from baseline.

The methods of the current invention provide reducing the risk of or delaying onset of end-organ damage in BKV positive patients by oral administration of Compound 12 or Compound 13 (or a pharmaceutically acceptable salt thereof). Subjects, who are BK viruric during the treatment period, may show beneficial effect due to treatment with Compound 12 or Compound 13 (or a pharmaceutically acceptable salt thereof), in reducing the incidence of renal dysfunction (creatinine elevations) by 1.5-4.5 fold. The incidence of renal dysfunction may be reduced by about 1.6, 1.7, 1.8, 1.9, 2.0, 2.1, 2.2, 2.3, 2.4, 2.5, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, or 4.5 fold. Among the BK-positive subjects of the current invention, there may be a 1.2-4.4 fold decrease of creatinine elevations or new onset heme+urinalyses. Among subjects who remain BK negative during the treatment period, the rates for either creatinine elevations or the combined analysis of creatinine or heme+urine may be numerically similar.

BKV has effects on renal function and the bladder (hematuria, cystitis, dysuria etc.). An analysis of routine laboratory values (serum creatinine elevations and the presence of new onset, confirmed hematuria) provides potential markers of BK effects in post-HSCT subjects. The method of the current invention provides measuring last value of creatinine, % increase over baseline level of creatinine, and heme+1 urinalysis during the treatment period among subjects treated with Compound 12 or Compound 13 (or a pharmaceutically acceptable salt thereof).

### Combination Therapy

The compounds or compositions provided herein may also be used in combination with an enhancer agent, with other active ingredients, or with an immunosuppressant agent. In certain embodiments, the compounds may be administered in combination, or sequentially, with another therapeutic agent or an enhancer. Such other therapeutic agents include those known for treatment, prevention, or amelioration of one or more symptoms associated with viral infections. It should be understood that any suitable combination of the compounds provided herein with one or more of the above-mentioned compounds and optionally one or more further pharmacologically active substances are considered to be within the scope of the present disclosure. In another embodiment, the compound provided herein is administered prior to or subsequent to the one or more additional active ingredients. In one embodiment, two or more of the antiviral agents disclosed herein are administered serially or in combination.

The amount of some enhancers can be selected using methods known in the art to enhance the bioavailability of the anti-viral agent. Any amount can be used that provides a desired response by some enhancers. The dosages may range, in a non-limiting example, from 0.001 mg to about 2000 mg of compound per kilogram of body weight per day, e.g., 0.01 to 500 mg/kg, or e.g., 0.1-20 mg/kg.

The co-administration of the compound or compositions provided herein with another agent may have a synergistic effect in treating BKV infection, reactivation of BKV, or preventing end organ damage or impairment in a subject infected with BKV. Specific examples of such combinations include, but are not limited to: Compound 12 or Compound 13 (or a pharmaceutically acceptable salt thereof) in combination with at least one immunosuppressant agents. Exemplary immunosuppressant agent include, but are not limited to, Daclizumab, Basiliximab, Tacrolimus, Sirolimus, Mycophenolate (as sodium or mofetil), Cyclosporine A, Glucocorticoids, Anti-CD3 monoclonal antibodies (OKT3), Antithymocyte globulin (ATG), Anti-CD52 monoclonal antibodies (campath 1-H), Azathioprine, Everolimus, Dactinomycin, Cyclophosphamide, Platinum, Nitrosurea, Methotrexate, Azathioprine, Mercaptopurine, Muromonab, IFN gamma, Infliximab, Etanercept, Adalimumab, Tysabri (Natalizumab), Fingolimodm and a combination thereof. In some embodiments, the pharmaceutical composition includes, e.g., Compound 12, Tysabri (natalizumab), and a pharmaceutically acceptable carrier.

In one embodiment, the pharmaceutical composition described herein comprises, e.g., Compound 12 or Compound 13 (or a pharmaceutically acceptable salt thereof) and one or more medication for treating viral infection, e.g., polyomavirus JC virus (“JCV”), that causes Progressive multifocal leukoencephalopathy (“PML”), in at least one pharmaceutically acceptable carrier. In one embodiment, one or more medication is selected from the group consisting of RITUXAN® (rituximab), RAPTIVA® (efalizumab), TYSABRI® (natalizumab), MYFORTIC® (mycophenolic acid), AVONEX® (interferon beta-1a), REMICADE® (infliximab), ENBREL® (etanercept), HUMIRA® (adalimumab), CELLCEPT® (mycophenolate mofetil), and a combination thereof in at least one pharmaceutically acceptable carrier.

### Effect of Food

In some embodiments, the pharmaceutical composition of the current embodiments, e.g., tablet or suspension, may be provided to a subject when the subject is either fasted or in fed conditions. In one embodiment, the composition comprising Compound 12 or Compound 13 (or a pharmaceutically acceptable salt thereof) may be provided to a subject having an empty stomach, e.g., after fasting for less than 24 hours but more than 12 hours, more than 11 hours, more than 10 hours, more than 8 hours, or more than 5 hours.

In other embodiments, the composition comprising Compound 12 or Compound 13 (or a pharmaceutically acceptable salt thereof) may be provided to a subject in combination with food or subsequent to having food. In one embodiment, Compound 12 or Compound 13 (or a pharmaceutically acceptable salt thereof) may be taken by a subject on an empty stomach.

### Patient Population

In certain embodiments, compounds of Formula (I), e.g., Compound 12 or Compound 13 (or a pharmaceutically acceptable salt thereof) (referred to as “Compound” in this section only), a composition comprising a Compound, or a combination therapy is administered to a mammal which is about 1 to 6 months old, 6 to 12 months old, 1 to 5 years old, 5 to 10 years old, 10 to 15 years old, 15 to 20 years old, 20 to 25 years old, 25 to 30 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old, or 95 to 100 years old.

In certain embodiments, a Compound, a composition comprising a Compound, or a combination therapy is administered to a human at risk for a virus infection. In certain embodiments, a Compound, a composition comprising a Compound, or a combination therapy is administered to a human with a virus infection. In certain embodiments, the patient is a human about 1 to 6 months old, 6 to 12 months old, 1 to 5 years old, 5 to 10 years old, 5 to 12 years old, 10 to 15 years old, 15 to 20 years old, 13 to 19 years old, 20 to 25 years old, 25 to 30 years old, 20 to 65 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100 years old.

In some embodiments, a Compound, a composition comprising a Compound, or a combination therapy is administered to a human infant. In other embodiments, a Compound, or a combination therapy is administered to a human child. In other embodiments, a Compound, a composition comprising a Compound, or a combination therapy is administered to a human adult. In yet other embodiments, a Compound, a composition comprising a Compound, or a combination therapy is administered to an elderly human.

All percentages and ratios used herein, unless otherwise indicated, are by weight. Other features and advantages of the present invention are apparent from the different examples. The provided examples illustrate different components and methodology useful in practicing the present invention. The examples do not limit the claimed invention. Based on the present disclosure the skilled artisan can identify and employ other components and methodology useful for practicing the present invention.

All patents, patent applications, and publications mentioned herein are hereby incorporated by reference in their entireties. However, where a patent, patent application, or publication containing express definitions is incorporated by reference, those express definitions should be understood to apply to the incorporated patent, patent application, or publication in which they are found, and not to the remainder of the text of this application, in particular the claims of this application.

### EXAMPLES

### Example 1

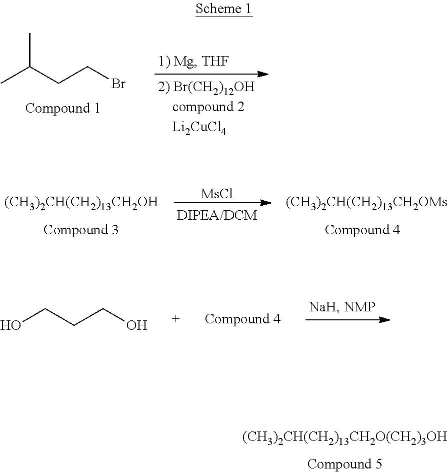

Synthesis of Compound 3:

To a solution 1-bromo-3-methyl butane (156.6 g, 1.037 mol) in Me-THF (1,252 ml) magnesium turnings (30.70 g, 1.263 mol) was added. The temperature began to increase from 21.7° C. to 50° C. after 4-5 minutes. A dry ice/acetone bath was utilized to control the temperature. Eventually, the temperature increased to 60° C. before cooling back down. At 40° C., a small chip of iodine was added. The solution was then heated to 61° C. and stirred for 2 hours. The heat was then removed and the mixture was cooled to 40° C. The reaction mixture was cooled further to −59° C. and to the mixture was added 12-bromo-1-dodecanol (50 g, 0.189 mol) in Me-THF (312.5 ml) so that the temperature never went above −55° C. Immediately following the addition of the 12-bromo-1-dodecanol, a dilithium tetrachlorocuprate(II) solution (0.1 M in THF, 103.68 ml) was added at once. The reaction was warmed to room temperature and stirred for 16 hours. Thin Layer Chromatography (TLC) (2:1 hexanes:ethyl acetate) indicated that the reaction was complete (R_{f} for starting material 0.48, R_{f} for product 0.58). The reaction was cooled to 0° C. and saturated NH₄Cl (750 ml) was slowly added to the reaction. The temperature increased to 22° C. over the course of the addition. The aqueous phase was back extracted with ethyl acetate (1,500 ml). The combined organics were washed with brine (750 ml), dried over MgSO₄, and filtered. The solution was concentrated in vacuo at 40° C. to afford 49 g (99%) of the desired material. The material was taken directly to next step.

Synthesis of Compound 4:

To a cold solution (0-5° C.) of Compound 3 (49 g, 0.191 mol) and N,N-Diisopropylethylamine (DIPEA) (27.41 g, 0.212 mol) in dichloromethane (490 ml) mesyl chloride (24.29 g, 0.212 mol) was added slowly to ensure the temperature never went above 5° C. The reaction was warmed to room temperature and stirred for 16 hours. To the reaction mixture mesyl chloride (0.065 mol, 7.4 g) and DIPEA (0.52 mol, 6.27 g) were added. The reaction was stirred for an additional 24 hours. The reaction was cooled to 6.5° C. and water (500 ml) was slowly added. The cooled reaction mixture was stirred for 2.5 hours. The DCM layer was separated, dried over Na₂SO₄, and filtered. The solution was concentrated in vacuo at 40° C. To the yellow residue 100 ml methanol was added. The solution was left at 4° C. for 30 minutes and a white solid precipitated. The solution with precipitated solid was filtered and air dried on filter for 16 hours. The filtrate was concentrated to one-half volume where additional solids precipitated, which was then filtered. The combined solids from the previous two steps were combined and triturated in methanol (200 ml) for 0.5 hours. The white solid was filtered and dried for 16 hours yielding 48 g (75%) of the desired product. This material was taken to the next step.

Synthesis of Compound 5:

To a cold (−5° C.) solution of 1, 3-propane diol (0.551 mol, 41.96 g) in NMP (225 ml) NaH (0.276 mol, 11.02 g) was added in small portions (˜0.5 g portions) over the course of 30 minutes. This mixture was warmed to room temperature and stirred for 30 minutes. To this solution Compound 4 (0.135 mol, 45 grams) dissolved in N-Methyl-2-pyrr olidone (NMP) (225 ml) was added. The reaction was stirred for 16 hours. TLC (2:1 hexanes:ethyl acetate) indicated the reaction was complete. The starting material had an R_{f} of 0.62 while the product had an R_{f} of 0.53. To the reaction mixture water (400 ml) and ethyl acetate (800 ml) were added. The organic and the aquous layers were separated. The organic layer was washed with water (2×500 ml). The solution was concentrated in vacuo at 40° C. The mixture was dried further by added methanol (2×200 ml) and concentrating in vacuo at 40° C. The step of drying and concentrating in methanol was also repeated with acetonitrile (2×200 ml) to afford a yellow oil. To the 59 g of yellow oil 120 ml of acetonitrile was added and this mixture was stirred at 0-5° C. for 1 hr. A waxy white solid, which was formed from the acetonitrile trituration, was filtered. The solid was immediately transferred to a 500 ml round bottom flask and dried on the rotary evaporator for 16 hours to yield 44 g of material. This dried material, Compound 5, was used in Scheme 2.

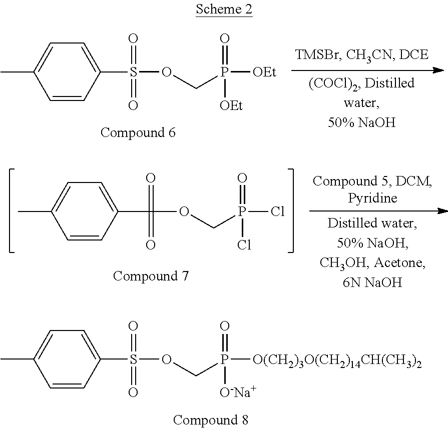

Synthesis of Compound 8:

To a solution of Compound 6 (0.146 mol, 47 g) in acetonitrile (423 ml) TMS-Br (0.327 mol, 50.01 g) was added, while maintaining an internal temperature of about 21.7-23.5° C. After the addition was complete, the internal temperature was adjusted to 55° C. and stirred for 2 hrs. After 2 hours, the acetonitrile and TMS-Br were removed via vacuum distillation at 40° C. to form a concentrate. To the concentrate dichloromethane (423 ml) was added to form a solution followed by oxalyl chloride (0.327 mol, 41.46 g), while maintaining an internal temperature of 25-40° C. After the addition of oxalyl chloride was complete, 2 drops of DMF were added. The reaction mixture was left to stir for 18 hours. The solution was concentrated in vacuo at an external temperature of 35° C. This material (Compound 7) was used to the next step.

To a cold (−8° C.) solution of Compound 5 (0.1272 mol, 40 g) and Compound 7 (0.1462 mol, 44.33 g) in dichloromethane (423 ml) pyridine (0.381 mol, 30.18 g) was added. This reaction mixture was stirred for 3 hours. TLC (2:1 hexanes:ethyl acetate) indicated that Compound 5 had been consumed. To the reaction mixture (cooled to 10° C.) 200 ml water was added. This mixture was stirred for 0.5 hr. The organic layer was then separated. To the organic layer 100 ml water and 75 ml methanol were added. The organic layer was again separated and concentrated in vacuo at 35° C. To the residue 200 ml acetone was added and concentrated until the residue was dry. To the residue 200 ml acetone was added and the pH was adjusted to about 9.04 using 6N NaOH (˜15 ml used). This mixture was left at 4° C. for 16 hours. 10 g of a white solid precipitated after the incubation period. The mixture with white solid precipitate was filtered. To the mixture an additional 300 ml acetone was added. The mixture was again left at 4° C. for 16 hours. Significant amounts of a tan solid precipitated after the additional incubation period. This mixture was filtered and dried to give ˜18 grams of product (Compound 8). NMR was performed, which confirmed that the product was Compound 8. This material was used in Scheme 3. Alternatively Compound 8 was prepared using the following procedure (procedure for Compound 8 on 20 g scale, purity by HPLC-ELSD)

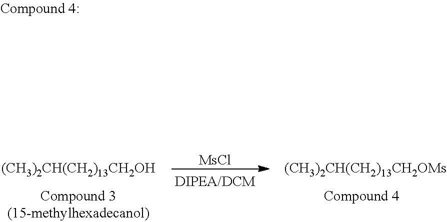

The reactor was charged with 15-methylhexadecanol (1.0 equiv., 13.4 kg, 52.25 moles), dichloromethane (172.2 kg), and N,N-disopropylethylamine (1.3 equiv., 8.8 kg, 67.92 moles). This mixture was cooled to −2.5° C. and to it was added methanesulfonyl chloride (1.3 equiv., 7.8 kg, 67.92 moles) over the course of 20 minutes while maintaining the temperature of ≦2° C. After the addition was complete, the temperature was adjusted to 0-5° C. and stirred for 30 minutes. The temperature was then adjusted to 20° C. and stirred for an additional 30 minutes. An in-process HPLC-ELSD analysis determined that the 15-methylhexadecanol had been consumed. To the reaction mixture was added water (40 kg). This was stirred for 2 hours and then allowed to settle for 0.5 hrs. The layers were separated and the organic layer was washed with water (20 kg) at 21.5° C. for 10 minutes. The layers were then allowed to settle for 1 hr. The layers were separated and the dichloromethane was concentrated at a temperature ≦35° C. until a volume of 18 L remained. To the reactor was charged ethanol (23.7 kg) and this mixture was agitated for 10 minutes at 20° C. The mixture was concentrated at a temperature of ≦55° C. until a volume of 18 L remained. This ethanol azeotrope was conducted one additional time. To the reactor was charged ethanol (15.8 kg). The solution was warmed to 28.7° C. and agitated for 1 hr. The solution was then cooled to 0° C. over the course of 37 minutes. It was then stirred at 0° C. for at least 17 hrs. The resulting solid was then filtered. The reactor was washed with ethanol (4.8 kg) and this was also transferred to the filter. The solid was transferred to a tray dryer and dried at a temperature of ≦35° C. until the loss on drying (LOD) was ≦1%. 16.1 kg (92%) obtained. Purity −99.3% by HPLC-ELSD (AUC).

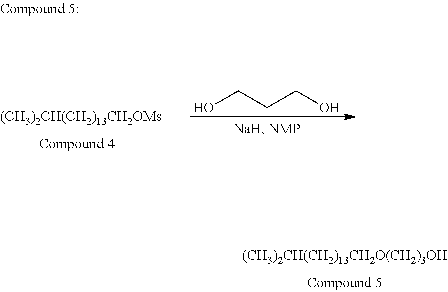

The reactor was charged with 1,3-propane diol (4.2 equiv., 15.4 kg, 201.6 moles) and NMP (65.4 kg). This mixture was agitated and cooled to −3.3° C. To this solution was added sodium hydride (60% dispersion in mineral oil, 2 equiv., 3.8 kg, 96.0 moles) portion wise over the course of 2 hours to ensure the temperature was maintained at ≦10° C. The temperature was adjusted to 20° C. and the solution was stirred for 1 hour. To the reactor was charged a solution of Compound 4 (1 equiv., 16.0 kg, 48.1 moles) in NMP (65.4 kg) at a rate to maintain the temperature at ≦35° C. The reactor that the Compound 4 solution was mixed was washed with NMP (21 kg) and this was also transferred to the reaction at a rate to maintain the temperature at ≦35° C. The reaction temperature was adjusted to 35° C. and this was agitated for 1 hour. The reaction temperature was then adjusted to 25° C. and this was agitated for 15 hours. An in-process HPLC-ELSD analysis determined Compound 4 was consumed. To the reaction mixture was charged heptanes (35 kg). The solution was then cooled to 3.6° C. and to it was added water (40 kg) over the course of 30 minutes while maintaining a temperature ≦20° C. The temperature was adjusted to 20° C. and stirred for 10 minutes. To the reactor was charged water (380 kg) and heptanes (70 kg). This was stirred for 10 minutes and then allowed to settle for 30 minutes. The layers were separated and the aqueous was re-extracted with heptanes (35 kg). The combined organics were agitated with brine (11.6 kg) for 10 minutes and then allowed to settle for 30 minutes. The layers were separated and the organic layer was dried over sodium sulfate (3.3 kg). This was agitated for 1 hour and then filtered. The reactor that contained the organic layer with sodium sulfate was washed with heptanes (20 kg) and this was also sent through the filter. The solvent was then concentrated at a temperature ≦50° C. until no further distillate was observed. To the reactor containing product was added acetonitrile (28.7 kg) and this was heated to 45° C. This was stirred for 10 minutes and then cooled to 0° C. The solution was then stirred at 0° C. for 1 hour. The resulting solid was then filtered. The reactor was washed with acetonitrile (4 kg) and this was also transferred to the filter. This acetonitrile recrystallization was repeated a second time. The product was dried on the filter for 1 hour. It was then transferred to a tray dryer and dried for approximately 72 hours at 22° C. when the LOD was determined to be ≦1.0%. 15.6 kg (103.3%) obtained. Purity 98.3% by HPLC-ELSD (AUC)

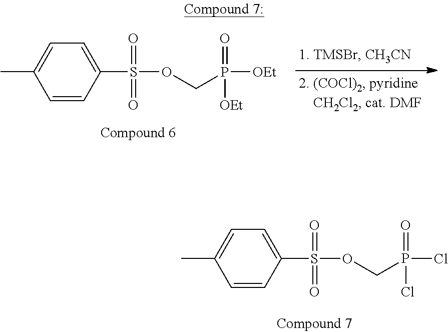 [SMILES: CCOP(=O)(COS(=O)(=O)c1ccc(C)cc1)OCC ; Cc1ccc(S(=O)(=O)OCP(=O)(Cl)Cl)cc1]

The reactor was charged with Compound 6 (1 equiv., 18.8 kg, 58.3 moles) and acetonitrile (70.7 kg). To this mixture was added bromotrimethylsilane (2.24 equiv., 20.0 kg, 130.6 moles) in one portion. The mixture was warmed to 55° C. and agitated for 3 hours. LC/MS determined that Compound 6 was consumed. The contents of the reaction were concentrated at a temperature of ≦50° C. until no further distillate was observed. To the reactor was charged 1,2-dichloroethane (25 kg) and this was concentrated at a temperature of ≦50° C. until no further distillate was observed. This was repeated one additional time. To the reactor was charged 1,2-dichloroethane (138.2 kg). The temperature was adjusted to 20° C. and to the solution was added oxalyl chloride (2.24 equiv., 16.6 kg, 130.6 moles) portionwise over the course of 30 minutes while maintaining the temperature at ≦30° C. The temperature of the reaction was adjusted to 55° C. and the mixture was agitated for 3 hours. 31P NMR analysis determined that the conversion of Compound 7 was only 57%. To the reaction was added a solution of dimethylformamide (85 g) in 1,2-dichloroethane (4 kg) over the course of 30 minutes while maintaining the temperature at ≦30° C. The reaction was then stirred at 25° C. for 1 hour. 31P NMR analysis determined that the conversion of Compound 7 was 98%. The contents of the reaction were concentrated at a temperature of ≦50° C. until the volume was below approximately 40 L. To the reactor was added 1,2-dichloroethane (25 kg) and this was concentrated at a temperature of ≦50° C. until the volume was below approximately 40 L. This 1,2-dichloroethane azeotrope was repeated 2 additional times to give a final volume of 40 L. HPLC determined purity was 90.4%. This material (Compound 7) was held at 20° C. under nitrogen until the next step (Compound 8).

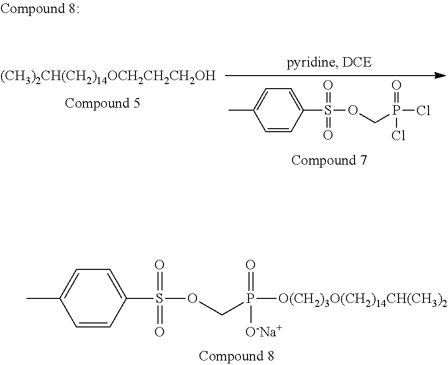

To the reactor containing the solution of Compound 7 (1.25 equiv., 17.7 kg, 58.3 moles) from above was added Compound 5 (1 equiv., 14.7 kg, 46.7 moles) and 1,2-dichloroethane (160.0 kg). This mixture was then cooled to 9° C. and to it was added a solution of pyridine (3 equiv., 11.1 kg, 140.1 moles) in 1,2-dichloroethane (10 kg) portion wise over the course of 30 minutes while maintaining an internal temperature of ≦15° C. The temperature never went above 10.8° C. The temperature was adjusted to 10° C. and stirred for 2 hours. An in-process HPLC-ELSD analysis determined that Compound 5 had been consumed. The reaction was cooled to 3° C. and to it was slowly added water (3 kg) over the course of 30 minutes while maintaining the temperature of ≦30° C. The temperature never went above 19° C. Over the next 40 minutes an additional 67 kg of water was added. The temperature was adjusted to 30° C. and the mixture was agitated for 16 hours. The agitation was stopped and the layers were allowed to settle for 23 hours. The layers were separated. To the organic layer was added a mixture of water (50 kg) and methanol (40 kg). This was agitated at 30° C. for 30 minutes and then allowed to settle for 30 minutes. The layers were separated and the methanol/water wash was repeated two times. To the organic layer was then added a mixture of water (50 kg)/methanol (40 kg)/6 N HCl (0.6 kg) and this was agitated at 30° C. for 30 minutes. The layers were allowed to settle for 30 minutes and then separated. The water/methanol/6N HCl wash was repeated 2 times. The organic layer was concentrated at ≦50° C. until no further distillate was observed. To the reactor containing product was added acetone (25 kg). The acetone was concentrated at ≦50° C. until no further distillate was observed. This azeotrope was repeated one more time. To the reactor containing product was added acetone (100.5 kg). To this was added 7.3 kg of a 6N NaOH solution. Adding this amount of the NaOH solution took the pH above the 9.75 mark, a solution of 1 N HCl was made and 0.4 kg of this HCl solution was added to the mixture to obtain a final pH of 9.68. The acetone solution was then cooled to 0° C. and to it was added 20 g of Compound 8 seed. This was agitated at 0° C. for 14.5 hours and then filtered. The reactor was washed with acetone (20 kg) at 0° C. and this was also directed to the filter. The solid product was then transferred back to the reactor and to it was added acetone (100 kg). This was agitated at 20° C. for 1 hour and then cooled to 0° C. The mixture was agitated at 0° C. for 2 hours and then filtered. The reactor was again washed with acetone (20 kg) at 0° C. and this was directed to the filter. The product was left to dry on the filter under vacuum for 13 hours. The product was transferred to a tray dryer and dried at ≦30° C. until the LOD was ≦1%. HPLC determined purity to be 95.1%. 19.3 kg (70.7%) obtained.

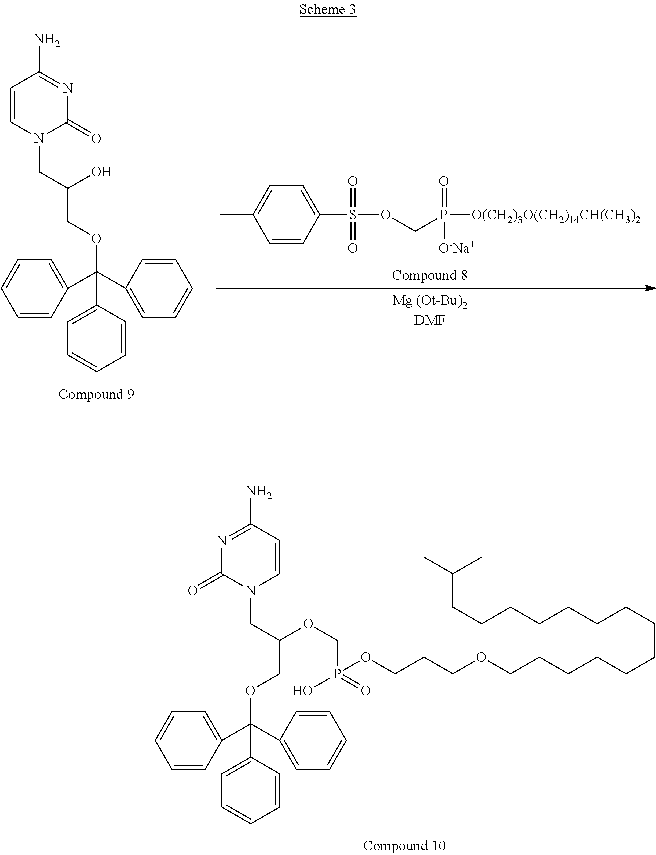

Synthesis of Compound 10: (Method A): The reactor was charged with anhydrous dimethylformamide (18 kg), Compound 9 (6 kg, 1 equiv., 14.04 mol), magnesium tert-butoxide (2.5 kg, 1.05 equiv., 14.74 mol) and Compound 8 (9.0 kg, 1.1 equiv., 15.44 mol). The reaction was heated to 80° C. and stirred for 3 hours. HPLC was used to monitor the reaction. When the Compound 9 was ≦20%, the reaction was cooled to 15° C. and to it was added isopropyl acetate (56 kg). To this was added an HCl solution (3 kg HCl in 36 kg water). The temperature was adjusted to 20° C. and the mixture was stirred for 1 hour. The stirring was stopped and the layers were allowed to settle for 1 hour. The layers were separated and the organic was washed with brine (10 kg NaCl in 40 kg water) two times. After the final separation, the isopropyl acetate was removed in vacuo at 40° C. until no more distillate was observed. To the reactor was added methanol (36.7 kg). This was removed in vacuo at 40° C. until no more distillate was observed. To the reactor was added methanol (36.7 kg). This solution was taken directly to the next step.

(Method B): Alternatively, Compound 9 (0.014 mol 6 grams), Compound 8 (0.029 mol, 17.17 g), magnesium di-tert butoxide (0.035 mol, 5.98 g), and DMF (25 ml) were added together in a flask and heated to 80° C. for 3 hours. At this point, HPLC indicated the reaction was complete. The reaction was cooled to r.t. and to it was added isopropyl acetate (45 ml) and 1 N HCl (40 ml). This was stirred for 0.5 hr. The organic layer was separated, washed with brine (2×41 ml), dried over Na₂SO₄, and concentrated in vacuo at 40° C. To the mixture was added methanol (2×100 ml) and this was concentrated further at 40° C. to produce Compound 10, which was then used in the next step.

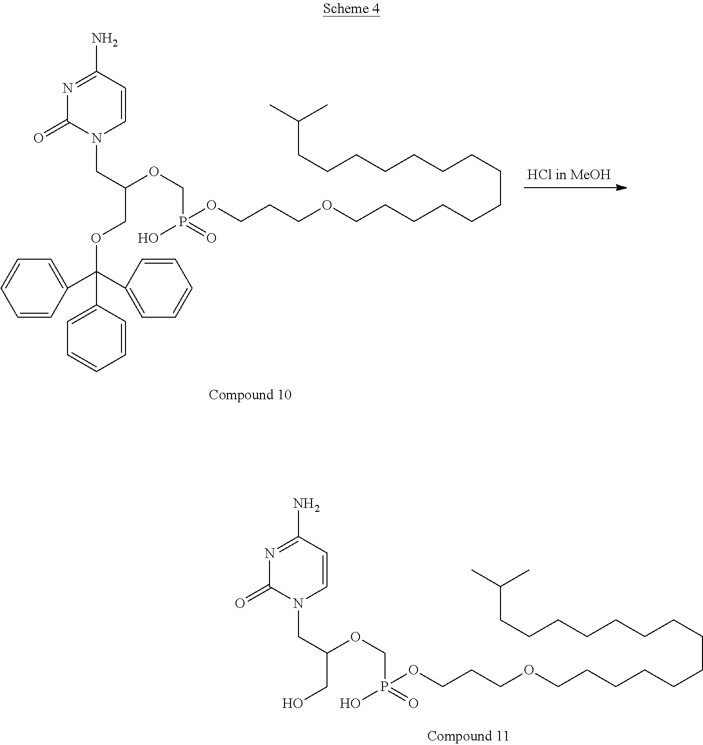 [SMILES: CC(C)CCCCCCCCCCCCCCOCCCOP(=O)(O)COC(COC(c1ccccc1)(c1ccccc1)c1ccccc1)Cn1ccc(N)nc1=O ; CC(C)CCCCCCCCCCCCCCOCCCOP(=O)(O)COC(CO)Cn1ccc(N)nc1=O]

Synthesis of Compound 11:

(Method A): The methanol solution from the previous step (Method A) was cooled to 0° C. and to it was bubbled in hydrogen chloride gas (1.6 kg). This solution was warmed to 15° C. and stirred for 2 hours. HPLC was used to monitor the reaction. When Compound 10 was ≦5%, the solid that had precipitated was filtered. This is not the product. The product is in filtrates. The reactor was washed with methanol (2.9 kg) and this was sent through the filter. The methanol filtrate was transferred back to the reactor and cooled to 5° C. To this solution was added water (54.3 kg) over the course of 30 minutes while maintaining a temperature of ≦30° C. Using a pH meter, a solution of 1N NaOH was added to the reaction filtrate until a pH of 2.5 was reached. The resulting solid was filtered. The reactor was washed with water (17.6 kg) and this was transferred to the filter. The solid was transferred back to the reactor and to it was added acetone (47.2 kg). This was stirred for 1 hour and then filtered under pressure. The cake was then washed with acetone (2×11.8 kg). The solid was transferred to tray dryers and dried under vacuum at ≦40° C. for 12 hours. After acetone removal is complete, the solid was transferred back to the reactor and to it was added methanol (41 kg). This was heated to reflux (65° C.) and stirred until a clear solution was obtained. Over the course of 6 hours, the solution was cooled to 0° C. and stirred for 2 hours. The solid was filtered. The reactor was washed with methanol (11.7 kg) at 0° C. and transferred to the filter. After no more filtrate was coming, the solid was transferred back to the reactor and to it was added methanol (51 kg). This was heated to reflux (65° C.) and stirred until a clear solution was obtained. Over the course of 6 hours, the solution was cooled to 0° C. and stirred 2 hours. The solid was collected through filtration. The reactor was again washed with methanol (11.7 kg) at 0° C. and this was transferred to the filter. The solid was then transferred to dryer trays and dried in vacuum oven at ≦40° C. for 24 hours. 5.6 kg (69%) of Compound 11 was obtained with purity of ≧92%.

(Method B) Alternatively, Methanol (70 ml) and 1.25 N HCl in methanol (0.0441 mol, 35.29 ml) was added to Compound 10 (12 g, 0.0147 mol) at room temperature. The reaction was stirred for 18 hours. HPLC indicated the reaction was complete. The white solid (trityl side product) was filtered. The filtrate was diluted with water (50 ml) and the pH was adjusted to 2.5 using 6 N NaOH. This was stirred for 1 hour at room temperature and filtered. The product (Compound 11) was slurried in acetone (2×100 ml) and filtered. The product was dried in the vacuum oven at room temperature for 24 hours. NMR confirmed product.

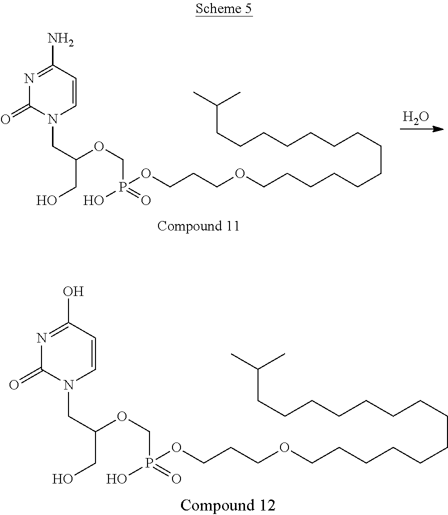

Synthesis of Compound 12: (Method A): The reactor is charged with Compound 11 (5.6 kg, 1 equiv., 9.73 mol) and water (84 kg). This was heated 90° C. and stirred for 20 hours. A clear solution was obtained. The reaction was heated to 99° C. and stirred for 96 hours. HPLC determined the Compound 11 was ≦1%. The reaction was cooled to 20° C. and to it was added sodium carbonate (1.5 kg, 1.5 equiv., 14.6 mol). This was stirred for 10 minutes. To this was added an ethyl acetate (65 kg)/2-propanol (6.3 kg) mixture. This was stirred for 5 minutes and allowed to separate for 20 minutes. The layers were separated. The aqueous (containing product) was transferred back to the reactor and to it was added an ethyl acetate (65 kg)/2-propanol (6.3 kg) mixture. This was again stirred for 5 minutes and allowed to separate for 20 minutes. The layers were separated. The aqueous (containing product) was transferred back to the reactor and cooled to 10° C. To this was carefully added a HCl solution (5.8 kg HCl in 4.8 kg water) keeping the temperature ≦25° C. The temperature was adjusted to 20° C. and to the aqueous solution was added an ethyl acetate (50.5 kg)/methanol (11.1 kg) solution. This was stirred for 10 minutes and allowed to separate for 20 minutes. The layers were separated. The aqueous was transferred back to the reactor and extracted 1 more time with an ethyl acetate (50.5 kg)/methanol (11.1 kg) solution. The organic layer was transferred back to the reactor and washed with a 0.5 M HCl solution (1 kg HCl in 19 kg water). This was stirred for 5 minutes and allowed to separate for 20 minutes. The lower aqueous layer was removed. To the reactor was added a 0.5 M HCl solution (1 kg HCl in 19 kg water) and methanol (1.6 kg). This was stirred for 5 minutes and allowed to separate for 20 minutes. The lower aqueous layer was removed. To the reactor was added a 0.5 M HCl solution (1 kg HCL in 19 kg water) and methanol (1.6 kg). This was stirred for 5 minutes and allowed to separate for 20 minutes. The lower aqueous layer was removed. The organic was vacuum distilled at a temperature ≦40° C. until no further distillate is observed. To the reactor is added methanol (40 kg) and this was vacuum distilled at a temperature ≦40° C. until no further distillate is observed. To the reactor is added methanol (60 kg) and charcoal (7 kg). This was heated to 62° C. and stirred for 20 minutes. It was then cooled to 20° C. and sampled for HPLC analysis. HPLC showed the purity to be 97.8%. The methanol solution was filtered over a pad of celite (6 kg) that had been washed with methanol (2×12 kg). The celite cake was washed further with methanol (2×50 kg) to ensure all product was removed. The methanol containing product was transferred to the reactor and vacuum distilled at a temperature ≦40° C. until no further distillate is observed. To the reactor was added acetone (20 kg) and this was distilled at a temperature ≦40° C. until no further distillate is observed. To the reactor was added acetone (20 kg) and this was distilled at a temperature ≦40° C. until no further distillate is observed. To the reactor was added acetone (20 kg). This solution was transferred to a 50 L rbf. The reactor was washed with additional acetone (8 kg) and transferred to the 50 L rbf. The acetone solution is cooled to −45° C. and stirred for 20 minutes. The product was then filtered and dried. 2.13 kg (38%) obtained. Purity ≧97%.

(Method B) Alternatively, water (90 ml) was added to Compound 11 (0.01563 mol, 9 g). This mixture was heated to 90° C. and stirred with a mechanical stirrer. After 1 week, HPLC indicated the reaction was greater than 95% complete. The mixture was cooled to room temperature. The pH was adjusted to 1-2 using 1N HCl. The aqueous solution was extracted with ethyl acetate (3×500 ml). The combined ethyl acetate extracts were dried over Na₂SO₄, filtered, and concentrated in vacuo at 40° C. to give 9 g of thick oil. This was triturated with acetone (100 ml) and cooled to 4° C. A white solid was filtered to give 5 g of product after drying. NMR confirmed that the product was Compound 12.

### Example 2

### Antiviral and Cytotoxicity Assays

Human Cytomegalovirus (HCMV) EC₅₀ in MRC-5 Cells:

Costar 96-well tissue culture plates were seeded with 20,000 MRC-5 cells/well in EMEM containing 2% Hyclone Standard Fetal Bovine Serum and 1% Hyclone Penicillin and Streptomycin. Outer wells were not used to minimize the edge-effect produced by extended incubations. Cells were inoculated with HCMV at an MOI of 0.01. Serial dilutions of test compounds were added to the cells and plates were incubated for 7 days at 37° C. in 5% CO₂. After 7 days of incubation, positive control wells showed cell morphology indicative of HCMV infection in 90 to 100% of the MRC-5 cells. Culture medium was gently removed from infected cells after the 7-day incubation. The cells were rinsed twice with ice-cold PBS, and then freeze/thawed once. Each well was incubated with 200 μL lysis buffer for 2 hours at 55° C. Lysis buffer included 0.5 mg/mL protease K, 50 mM KCl, 10 mM Tris-Cl pH 8.0, 2.5 mM MgCl₂, 0.45% IGEPAL, and 0.45% Tween-20 dissolved in DEPC-treated water. Intracellular CMV DNA was measured by quantitative polymerase chain reaction (qPCR) using forward and reverse HCMV PCR primers, and a FAM-labeled probe. Absolute quantitation of viral copy number was performed using a standard curve with dilutions of a HCMV DNA amplicon containing sequences homologous to the amplified fragment. The following qPCR amplification conditions were used: 1 cycle at 95° C. for 10 minutes, followed by 45 cycles of 95° C. for 15 seconds and 60° C. for 60 seconds. The qPCR reactions were performed using an Applied Biosystems 7500 real Time PCR System. Gen 5 software (BioTek Instruments, Inc.) was used to calculate the concentration which inhibited the viral DNA levels of HCMV-infected MRC-5 cells by 50% (EC₅₀).

BKV EG₅₀ in VERO Cells:

Costar 96-well tissue culture plates were seeded with 10,000 Vero cells/well in DMEM containing 2% Hyclone Standard Fetal Bovine Serum (FBS, Cat 5H30088.03) and 1% Hyclone Penicillin and Streptomycin. Outer wells were not used to minimize the edge-effect produced by extended incubations. Cells were inoculated with 115 BKV DNA copies/cell (ATCC, Gardner strain). Serial dilutions of test compounds were added to the cells and plates were incubated for 10 days at 37° C. in 5% CO₂. After the 10-day incubation, 50 μL supernatant was mixed with 50 μL 2× lysis buffer that provided a final concentration 0.5 mg/mL protease K, 50 mM KCl, 10 mM Tris-Cl pH 8.0, 2.5 mM MgCl2, 0.45% IGEPAL, and 0.45% Tween-20 dissolved in DEPC-treated water. Each plate was incubated for 2 hours at 55° C. Supernatant BKV DNA was measured by quantitative polymerase chain reaction (qPCR) using forward and reverse BKV PCR primers, and a FAM-labeled probe. Absolute quantitation of viral copy number was performed using a standard curve with dilutions of a BKV DNA amplicon containing sequences homologous to the amplified fragment. The following qPCR amplification conditions were used: 1 cycle at 95° C. for 10 minutes, followed by 45 cycles of 95° C. for 15 seconds and 60° C. for 60 seconds. The qPCR reactions were performed using an Applied Biosystems 7500 real Time PCR System. Gen 5 software (BioTek Instruments, Inc.) was used to calculate the concentration which inhibited the viral DNA levels of BKV-infected Vero cells by 50% (EC₅₀).

Cytotoxicity (CC₅₀) in Vero Cells:

Costar 96-well tissue culture plates were seeded with 10,000 Vero cells (ATCC)/well in DMEM (ATCC, Cat 30-2002) containing 2% Hyclone Standard Fetal Bovine Serum (FBS, Cat 5H30088.03) and 1% Hyclone Penicillin and Streptomycin (Cat SV30010). Outer wells were not used to minimize the edge-effect produced by extended incubations. Serial dilutions of test compounds were added to the cells and plates were incubated for 7 days at 37° C. in 5% CO₂. After 7 days of incubation 40 μL of Cell Titer 96® Aqueous MTS Reagent (Promega, G111) was added as directed by manufacturer to the 200 iut media in each well and incubated at 37° C. until the untreated-cell controls developed an 490 nm absorbance between 1.1 and 1.8. The final absorbance readings were read using a BioTek Synergy 2, and Gen 5 software (BioTek Instruments, Inc.) was used to calculate the concentration which inhibited Vero cells by 50% (CC₅₀).

Cytotoxicity (CC₅₀) in MRC-5 Cells:

Costar 96-well tissue culture plates were seeded with 20,000 MRC-5 cells (ATCC)/well in EMEM (ATCC, Cat 30-2003) containing 2% Hyclone Standard Fetal Bovine Serum (FBS, Cat 5H30088.03) and 1% Hyclone Penicillin and Streptomycin (Cat SV30010). Outer wells were not used to minimize the edge-effect produced by extended incubations. Serial dilutions of test compounds were added to the cells and plates were incubated for 7 days at 37° C. in 5% CO₂. After 7 days of incubation 40 μL of Cell Titer 96® Aqueous MTS Reagent (Promega, G111) was added as directed by manufacturer to the 200 iut media in each well and incubated at 37° C. until the untreated-cell controls developed an 490 nm absorbance between 1.1 and 1.8. The final absorbance readings were read using a BioTek Synergy 2, and Gen 5 software (BioTek Instruments, Inc.) was used to calculate the concentration which inhibited MRC-5 cells by 50% (CC₅₀).

Cytotoxicity (CC₅₀) in MT4 Cells:

Costar 96-well tissue culture plates were seeded with 5,000 MT4 cells (NIH AIDS Reagents Program)/well in RPMI (Lonza, Cat 12-11F) containing 10% Hyclone Standard Fetal Bovine Serum (FBS, Cat 5H30088.03), 1% Hyclone Penicillin and Streptomycin (Cat SV30010), and 2 mM Lonza L-Glutamine (Cat 17-605E). Outer wells were not used to minimize the edge-effect produced by extended incubations. Serial dilutions of test compounds were added to the cells and plates were incubated for 6 days at 37° C. in 5% CO₂. After 6 days of incubation 40 μL of Cell Titer 96® Aqueous MTS Reagent (Promega, G111) was added as directed by manufacturer to the 200 μL media in each well and incubated at 37° C. until the untreated-cell controls developed an 490 nm absorbance between 1.1 and 1.8. The final absorbance readings were read using a BioTek Synergy 2, and Gen 5 software (BioTek Instruments, Inc.) was used to calculate the concentration which inhibited MT4 cells by 50% (CC₅₀).

Cells culture and virus strains. Human foreskin fibroblast (HFF) cells were prepared from human foreskin tissue obtained from the University of Alabama at Birmingham tissue procurement facility with approval from its IRB. The tissue was incubated at 4° C. for 4 h in cell culture media consisting of minimum essential media (MEM) with Earle's salts supplemented with 10% fetal bovine serum (FBS) (Hyclone, Inc. Logan Utah), and standard concentrations of L-glutamine, fungizone, and vancomycin. Tissue was then placed in phosphate buffered saline (PBS), minced, rinsed to remove the red blood cells, and resuspended in trypsin/EDTA solution. The tissue suspension was incubated at 37° C. and gently agitated to disperse the cells, which were then collected by centrifugation. Cells were resuspended in 4 ml media and placed in a 25 cm² tissue culture flask and incubated at 37° C. in a humidified CO₂ incubator for 24 h. The media was then replaced with fresh media and the cell growth was monitored daily until a confluent cell monolayer was formed. The HFF cells were then expanded through serial passages in standard growth medium of MEM with Earl's salts supplemented with 10% FBS, L-glutamine, penicillin, and gentamycin. The cells were passaged routinely and used for assays at or below passage 10.

Vero cells were obtained from American Type Culture Collection (ATCC, Manassas, Va.), and were maintained in standard growth medium of MEM with Earl's salts supplemented with 10% FBS, L-glutamine, penicillin, and streptomycin.

Akata cells latently infected with EBV were obtained from John Sixbey. The GS strain of HHV-6A was obtained through the NIH AIDS Research and Reference Reagent Program.

Antiviral Assays:

Each experiment that evaluated the antiviral activity of the compounds included both positive and negative control compounds to ensure the performance of each assay. Concurrent assessment of cytotoxicity was also performed for each study at equivalent levels of compound exposure. When sufficient material was available, multiple assays were performed for each compound evaluation to obtain statistical data.

Plaque reduction assays for HSV-1, VZV and HCMV. Monolayers of HFF cells were prepared in six-well plates and incubated at 37° C. for 2 d to allow the cells to reach confluency. Media was then aspirated from the wells and 0.2 ml of virus was added to each of three wells to yield 20-30 plaques in each well. The virus was allowed to adsorb to the cells for 1 h and the plates were rocked gently every 15 min to redistribute the media. Compounds were diluted in maintenance cell culture media consisting of MEM with Earl's salts supplemented with 2% FBS, L-glutamine, penicillin, and gentamycin. Solutions ranging from 300 μM to 0.1 μM were added to duplicate wells and the plates were incubated for various times, depending on the virus used. The plaque reduction assay with HSV-1 strain F was performed in a similar manner but with Vero cells infected one day after plating. Final FBS concentration in this assay was 5%. For HSV-1 and -2, the monolayers were stained with 1% crystal violet in 20% methanol and the unbound dye removed by washing with dH₂O. For assays with HCMV and VZV, the cell monolayer was stained with 1% Neutral Red solution for 4 h then the stain was aspirated and the cells were washed with PBS. For all assays, plaques were enumerated using a stereomicroscope and the concentration of compound that reduced plaque formation by 50% (EC₅₀) was interpolated from the experimental data.

DNA Hybridization Assays for EBV, HHV-6a, and HHV-6B.

Assays for EBV were performed in Akata cells that were induced to undergo a lytic infection with 50 μg/ml of a goat anti-human IgG antibody by standard methods. Experimental compounds were diluted in round bottom 96-well plates to yield concentrations ranging from 20 to 0.016 μM. Akata cells were added to the plates at a concentration of 4×10⁴ cells per well and incubated for 72 h. For HHV-6 assays, compounds were serially diluted in 96-well plates then 1×10⁴ uninfected HSB-2 or Molt-3 cells were added to each well. Infection was initiated by adding HHV-6A infected HSB-2 cells, or HHV-6B infected Molt-3 cells, at a ratio of approximately 1 infected cell for every 10 uninfected HSB-2 cells or Molt-3 cells respectively and incubated for 7 d at 37° C.

For all assays, 100 μ1 of denaturation buffer (1.2M NaOH, 4.5M 80 NaCl) was added to each well to denature the DNA and a 50 μA aliquot was aspirated through an Immobilon nylon membrane (Millipore, Bedford, Mass.) using a Biodot apparatus (Bio-Rad, Hercules, Calif.). The membranes were then allowed to dry before equilibration in DIG Easy Hyb (Roche Diagnostics, Indianapolis, Ind.) at 56° C. for 30 min. Specific digoxigenin (DIG)-labeled probes were prepared for each virus according to the manufacturer's protocol (Roche Diagnostics). For EBV, primers 5′-CCC AGG AGT CCC AGT AGT CA-3′ and 5′-CAG TTC CTC GCC TTA GGT TG-3 amplified a fragment corresponding to coordinates 96802-97234 in EBV genome (AJ507799). A specific HHV-6 DIG labeled probe was prepared using primers 5′-CCT TGA TCA TTC GAC CGT TT-3′ and 5′-TGG GAT TGG GAT TAG AGC TG-3′ to amplify a segment of ORF2 (coordinates 37820-38418 in X83413). Membranes with EBV DNA were hybridized overnight at 56° C. followed by sequential washes in 0.2×SSC with 0.1% SDS and 0.1×SSC with 0.1% SDS at the same temperature. For HHV-6A and HHV-6B blots, the probe was allowed to hybridize overnight at 42° C. and the blots were rinsed at the same temperature with 0.2×SSC with 0.1% SDS and 0.1×SSC with 0.1% SDS. Detection of specifically bound DIG probe was performed with anti-DIG antibody using the manufacturer's protocol (Roche Diagnostics). An image of the photographic film was captured and quantified with QuantityOne software (Bio-Rad) and compound concentrations sufficient to reduce the accumulation of viral DNA by 50% (EC₅₀), were interpolated from the experimental data.

(v) Influenza Virus

Cell-based assays. For dose-response curves, individual drugs were added to MDCK cells in 96-well microplates (8×10⁴ cells/well) using three wells for each concentration used. The compounds were added at the following concentrations: oseltamivir carboxylate at 0, 0.000032, 0.0001, 0.00032, 0.001, 0.0032, 0.01, 0.032, 0.1, 1.0, 10.0 and 100 μg/ml; amantadine and ribavirin at 0, 0.001, 0.0032, 0.01, 0.032, 0.1, 0.32, 1, 3.2, 10, 32 and 100 μg/ml. Untreated wells of infected cells (virus controls) and uninfected cells (cell controls) were included on each test plate. At three days post-infection, the virus control wells exhibited 100% cytopathology. The extent of viral cytopathology in each well was determined microscopically by inspection and by staining with neutral red (NR). Briefly, the cells were stained with 0.011% NR diluted in MEM to determine cell viability. Two hours later the plates were processed for quantification of NR uptake into viable cells. The amount of NR taken up by cells was determined spectrophotometrically.

Cytotoxicity Assays:

Every antiviral assay included a parallel cytotoxicity assay with the same cells used for each virus, the same cell number, the same drug concentrations, and the same incubation times to provide the same drug exposure. To ensure that the cytotoxicity of all compounds could be compared directly, we also performed a standard neutral red uptake cytotoxicity assay for all compounds in confluent HFF cells with a 7 d incubation period.

(i) Neutral Red Uptake Cytotoxicity Assays.

Each compound was evaluated in a standard cytotoxicity assay by standard methods. Briefly, HFF cells were seeded into 96-well tissue culture plates at a 2.5×10⁴ cells/well in standard tissue culture medium. After 24 h of incubation, medium was replaced with maintenance cell culture medium and compounds were added to the first row and then 5-fold serial dilutions were then used to generate a series of compound concentrations with a maximum of 300 μM. Assay plates were incubated for 7 d, and 100 μl of a 0.66 mg/ml neutral red solution in PBS was added to each well and the plates incubated for 1 h. The stain was then removed, the plates rinsed with PBS and the dye internalized by viable cells was solubilized in PBS supplemented with 50% ethanol and 1% glacial acetic acid. The optical density was then determined at 550 nm and CC₅₀ values were interpolated from the experimental data.

For all plaque reduction assays in HFF cells, neutral red cytotoxicity assays were performed on a parallel set of 6-well plates containing uninfected HFF cells that received the same compound concentrations as used for the antiviral assays. The cytotoxicity plates were removed from the incubator on the same day as each antiviral assay and the cell monolayer was stained for 6 h with 2 ml of a neutral red solution at a concentration of 0.165 mg/ml in PBS. The dye was then removed, residual dye rinsed from the cells with PBS, and cell monolayers were inspected visually for any signs of toxicity. Cytotoxicity assay with Vero cells was performed with drug concentrations ranging from 1 μM to 1 mM. The cell viability was determined using CellTiter 96® Aqueous One Solution Cell Proliferation Assay (Promega) according to manufacturer's instructions.

(ii) Cytotoxicity in Lymphocyte Assays.

Cell viability in all assays with lymphocytes was assessed with the CellTiter-Glo Luminescent Cell Viability Assay (Promega). Briefly, assay plates were incubated at ambient temperature for 30 min then 50 μ1 of CellTiter-Glo reagent was added to each well and the plates were mixed for 2 min on an orbital shaker to lyse the cells. Plates were then incubated for an additional 10 min at ambient temperature and the luminescence was quantified on a luminometer. Standard methods were used to calculate drug concentrations that inhibited the proliferation of Akata, HSB-2, BCLB-1, or Molt-3 cells by 50% (CC₅₀).

(iii) Cell Proliferation Assays.

The inhibition of HFF cell proliferation was used to refine estimates of cytotoxicity for some compounds and was performed according to a standard procedure used in the laboratory. Cells were seeded at a low density into six-well plates using 2.5×10⁴ cells/well and standard culture medium. After 24 h, the medium was aspirated, and a range of compound solutions in the growth medium was prepared starting at 300 μM, and added to duplicate wells. The plates were incubated for 72 h at 37° C., the cells were then dislodged with trypsin and counted on a Beckman Coulter Counter. Compound concentrations that reduced cell proliferation by 50% were interpolated from experimental data.

TABLE 2
Activity of Compound 12 against Herpes Simplex Virus 1 (Strain E-
377) and HCMV (Strain AD169) in human foreskin fibroblast cells

| | HSV-1 | HSV-1 | | HCMV | HCMV | |
|---|---|---|---|---|---|---|
| | EC₅₀, | CC₅₀, | HSV-1 | EC₅₀, | CC₅₀, | HCMV |
| Compound | μM | μM | SI₅₀ | μM | μM | SI₅₀ |
| Compound 12 | >50 | >50 | 1 | 0.052 | >50 | >961 |
| Acyclovir | 2.2 | >100 | >45 | | | |
| Ganciclovir | | | | 1.5 | >100 | >67 |

TABLE 3
Activity of Compound 12 and Hexadecyloxypropyl-Cidofovir
(HDP-CDV) against HCMV UL54 resistant mutants in
human foreskin fibroblast cells

| | Compound | Compound | HDP- | HDP- |
|---|---|---|---|---|
| | 12 | 12 | CDV | CDV |
| | EC₅₀ | EC₉₀ | EC₅₀ | EC₉₀ |
| Virus | μM | μM | μM | μM |
| AD169 | 0.052 | 0.15 | 0.001 | |
| D542E | 5.41 | >10 | 0.0295 | 0.1529 |
| (HDP-CDV^{R}) | | | | |
| GDG^{R}P53 | 0.171 | 1.552 | 0.0053 | 0.0316 |
| 4955^{R} | 0.143 | 0.372 | 0.0013 | 0.006 |

TABLE 4
Activity of Compound 12 against Vaccinia Virus
(Strain Copenhagen) and Cowpox Virus (Strain
Brighton) in Human Foreskin Fibroblast Cells

| | VACV | VACV | | CPXV | CPXV | |
|---|---|---|---|---|---|---|
| | EC₅₀, | CC₅₀, | VACV | EC₅₀, | CC₅₀, | CPXV |
| Compound | μM | μm | SI₅₀ | μM | μM | SI₅₀ |
| Compound | >10 | >10 | 1 | >10 | >10 | 1 |
| 12-001 | | | | | | |
| Cidofovir | 22.42 | >300 | >13 | 30.52 | >300 | >10 |

TABLE 5
Activity of Compound 12 against Strains of Influenza virus in
Madin-Darby Canine Kidney (MDCK) cells

| | A/CA/10/2009 | B/FL/4/2006 | Cytotoxicity |
|---|---|---|---|
| Compound | (EC₅₀, μM) | (EC₅₀, μM) | (CC₅₀, μM) |
| Compound 12 | >4 | >4 | 17.18 |
| Oseltamivir | 0.17 | 16.25 | >20 |

TABLE 6
Activity of Compound 12 against Epstein-Barr virus in Akata cells

| | Efficacy | Cytotoxicity |
|---|---|---|
| | DNA Hybridization Assay | CellTiter-GloAssay |
| Compound | (EC₅₀, μM) | (CC₅₀, μM) |
| Compound 12 | >10 | 18 |
| Acyclovir | 7.0 | >100 |

TABLE 7
Activity of Compound 12 and HDP-CDV against HCMV in MRC-5
cells and BKV in Vero cells

| | CMV | BKV | MT4 | CMV - SI | BKV - SI |
|---|---|---|---|---|---|
| | EC₅₀ | EC₅₀ | CC₅₀ | (MT4 CC₅₀/ | (MT4 CC₅₀/ |
| Compound | (μM) | (μM) | (μM) | EC₅₀) | EC₅₀) |
| Compound 12 | 0.027 | 1.55 | 25.6 | 948 | 16.5 |
| | (n = 4) | (n = 2) | (n = 7) | | |
| HDP-CDV | 0.0004 | 0.018 | 0.060 | 150 | 3.3 |
| | (n = 8) | (n = 11) | (n = 6) | | |

TABLE 8
In Vitro Antiviral Activity of Compound 12

| Virus | EC₅₀ (μM) | CC₅₀ (μM) | SI | Cell line |
|---|---|---|---|---|
| HCMV | 0.017, n = 12 | 50, n = 4 | 2941 | MRC5 |
| MCMV | 1.6, n = 2 | 60, n = 1 | 37.5 | MEF |
| HHV-8 | <0.08, n = 1 | >10, n = 1 | >125 | BCBL-1 |
| HCV | 0.57, n = 1 | >20, n = 1 | >35 | Huh |
| BKV | 2.1, n = 4 | 93, n = 1 | 44 | Vero |
| HBV | 3, n = 1 | 34, n = 1 | 11 | 2.2.15 |
| Poliovirus 3 | 3.3, n = 1 | 29, n = 1 | 9 | Vero 76 |
| HHV-6B | 4.7, n = 1 | 8.4, n = 1 | ~2 | MOLT-3 |
| Ebola | 1.0, n = 1 | 36.9, n = 1 | 37 | Huh |

TABLE 9
In Vitro Activity Comparison (Mean XC₅₀)

| | Compound | | |
|---|---|---|---|
| | Brincidofovir (BCV) | Compound 12 | Compound 24 |
| HCMV | 0.0003 | 0.017 | 0.008 |
| EC₅₀ (uM) | N = 9 | N = 10 | N = 4 |
| MT4 | 0.06 | 24.7 | 13.5 |
| cytotoxicity | N = 10 | N = 8 | N = 6 |
| CC₅₀ (uM) | | | |

Taken together, these data demonstrate that Compound 12 has an improved efficacy/toxicity ratio. The data on UL54 HCMV resistant variants shows that Compound 12 efficacy is reduced by mutations in this gene which reduce efficacy for HDP-CDV, suggesting that Compound 12 is anabolized to the triphosphate equivalent and acts as an alternative substrate inhibitor for the CMV polymerase UL54.

### Example 3

### CMV Clinical Studies

Clinical Studies of Compound 12 and Compound 13 are performed. A placebo-controlled, dose-escalating trial in HSCT CMV (R+) recipients, evaluating the ability of Compound 12 or Compound 13 to prevent or control CMV infection is carried out. Several cohorts are established in which participants or subjects receive either placebo or the Compound 13 orally, in doses ranging from 40-1000 mg daily or weekly (QW) to 40-1000 mg twice weekly (BIW), e.g., receive 200 mg once weekly or 100 mg twice weekly. Subjects who are post-HSCT are enrolled at the time of engraftment and randomized to Compound 13 or placebo (3 to 1 ratio) and receive blinded therapy until approximately 100 day post-transplantation. Compound 12 or Compound 13 doses are between 40-1000 mg daily or QW and 40-1000 mg BIW, e.g., 40 mg daily or QW, 100 mg daily or QW, 200 mg daily or QW, 200 mg BIW and 100 mg BIW. Subjects who develop CMV disease or CMV infection requiring pre-emptive therapy with local standard of care are discontinued from blinded therapy and followed for 4 weeks. Subjects who complete treatment with blinded therapy are followed for 8 weeks post-therapy.

Patients receive the study drug for 9 to 11 weeks, depending on the day of randomization after transplantation, such that the study drug is discontinued on week 13 after transplantation in patients who completed the study drug. Weekly measurement of plasma CMV DNA levels by means of a PCR assay is performed by a central laboratory while patients are receiving the study drug. If CMV disease develops or if patients require treatment with a drug against CMV infection because CMV DNA is detected in plasma or for another reason, the study drug is discontinued and patients are treated according to study-site practices.

The primary efficacy end point is the failure to prevent progressive CMV infection, defined as CMV disease or a plasma CMV DNA level greater than 200 copies per milliliter, detected at a central laboratory within 1 week after the last dose of the study drug. Study treatment (with either Compound 12 or Compound 13 or placebo) is considered to be successful if patients have an end-of-study plasma CMV DNA level of 200 copies per milliliter or less and does not have confirmed CMV disease, even if a particular weekly measurement is greater than 200 copies per milliliter during the study-drug administration and then decreases again. If patients discontinue the study drug to start treatment for CMV infection or for other reasons, but the plasma CMV DNA level is 200 copies per milliliter or less and CMV disease is not confirmed, treatment with Compound 12 or Compound 13 is considered to be successful. Pre-specified secondary end points include the occurrence of CMV infection or an increase in the plasma CMV DNA level in patients who are negative or positive for CMV DNA at baseline (either at screening or on the first day of study-drug administration), rates of and reasons for discontinuation of the study drug, the use of antiviral agents to treat CMV events, and trough levels of Compound 12 or Compound 13 and cidofovir. Safety end points include all adverse events, changes in laboratory values and electrocardiographic assessments, and death from any cause.

### Example 4

### BKV Clinical Studies

A 9-11 week randomized, placebo-controlled, double-blind, dose-escalation clinical study (40-1000 mg QW and 40-1000 mg BIW, e.g., 40 mg QW, 100 mg QW, 200 mg QW, 200 mg BIW, and 100 mg BIW) of Compound 12 or Compound 13 for the prevention of BKV infection post-HCT is performed. Treatment is initiated at the time of engraftment and continued until Week 13 post-HCT.

### A Study of BKV Associated End-Organ Damage

The subjects in the cohort are analyzed for incidence of BKV infection and effect on end-organ complications. BK viruria is measured at every visit and viremia assessed if viruria is present. Data from the study are analyzed to assess whether Compound 12 or Compound 13 has an effect of BKV infection end-organ diseases. Microscopic hematuria is defined as confirmed heme positive urinalyses; renal impairment is defined as having an elevated creatinine (≧120 μmol/L) on the last measurement during treatment that was also ≧25% increased from baseline.

In a clinical study, among the subjects enrolled in the study, some of the subjects receive placebo and others receive Compound 12 or Compound 13, at various doses.

Definitions and Methods

Clinically meaningful end organ effects were defined as follows:
Microscopic hematuria—at least 1+ heme noted on urinalysis (dipstick)
New onset hematuria—at least 1+ heme (confirmed by a consecutive measure of ≧Trace), occurring during treatment only
Renal dysfunction at end of treatment—serum creatinine ≧120 μM (≧1.36 mg/dl) at the end of treatment
New onset renal dysfunction—serum creatinine ≧120 μM (≧1.36 mg/dl) at the end of treatment AND at least 25% greater than baseline serum creatinine

Subjects are tabulated according to treatment group (pooled Compound 13 versus placebo) and BKV status (viruria positive or negative any time during treatment). Pairwise comparisons are performed using a Fisher's exact test. Data are pooled for Compound 12 versus placebo groups due to the limited sample size.

To explore the impact of BKV infection on emergence of symptoms, the incidence of urinary AEs containing the term BKV in subjects with BKV infection prior to dosing (i.e., BKV PCR positive at Baseline) is first analyzed.

To further explore the impact of Compound 12 on hemorragic cystitis emergence, the incidence of treatment emergent hematuria is explored.

### EQUIVALENTS

The invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A compound represented by Formula (I) as follows: 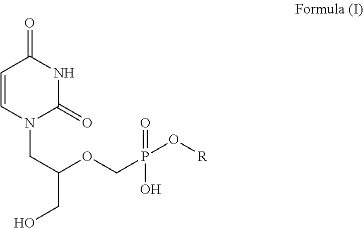
wherein R is:

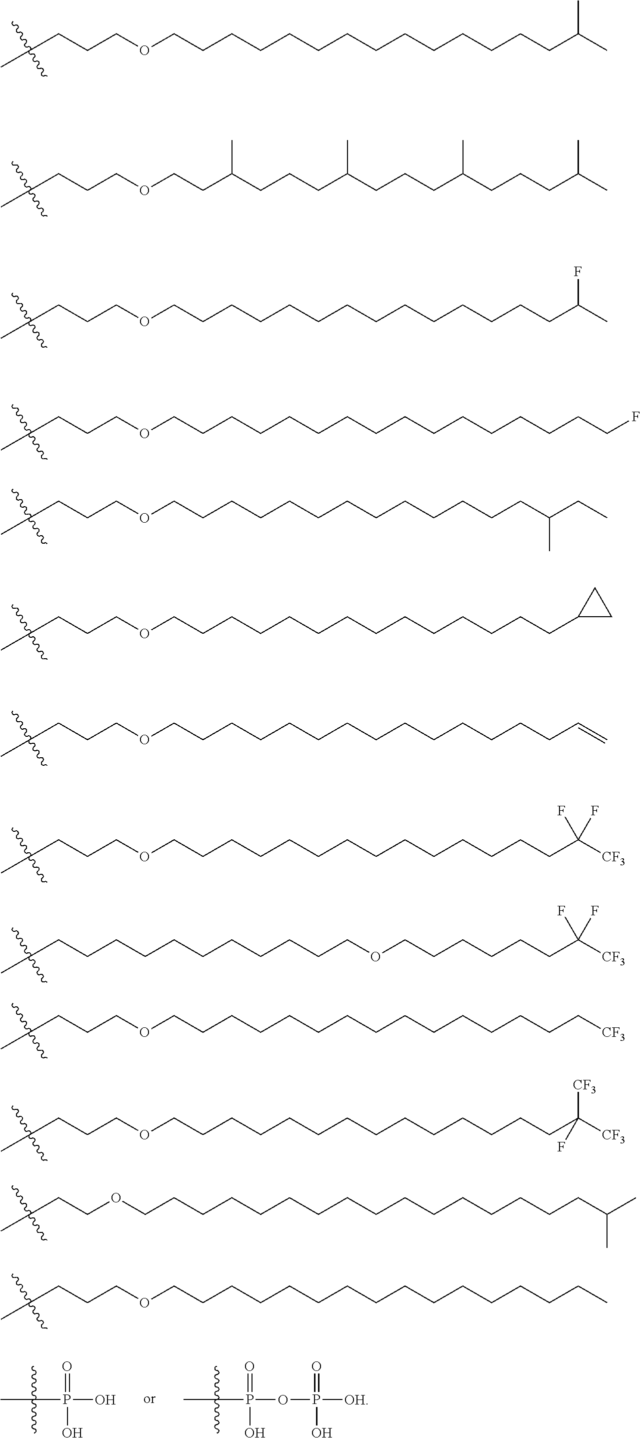

2. The compound of claim 1, wherein R is:

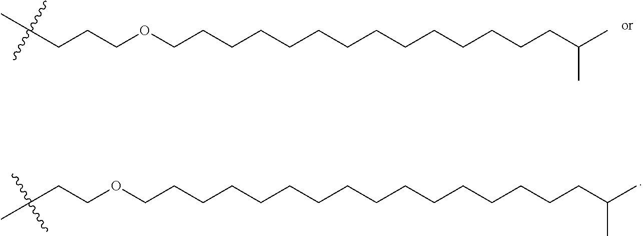 [SMILES: *CCCOCCCCCCCCCCCCCCC(C)C ; *CCOCCCCCCCCCCCCCCCCC(C)C]

3. A compound selected from the group consisting of: 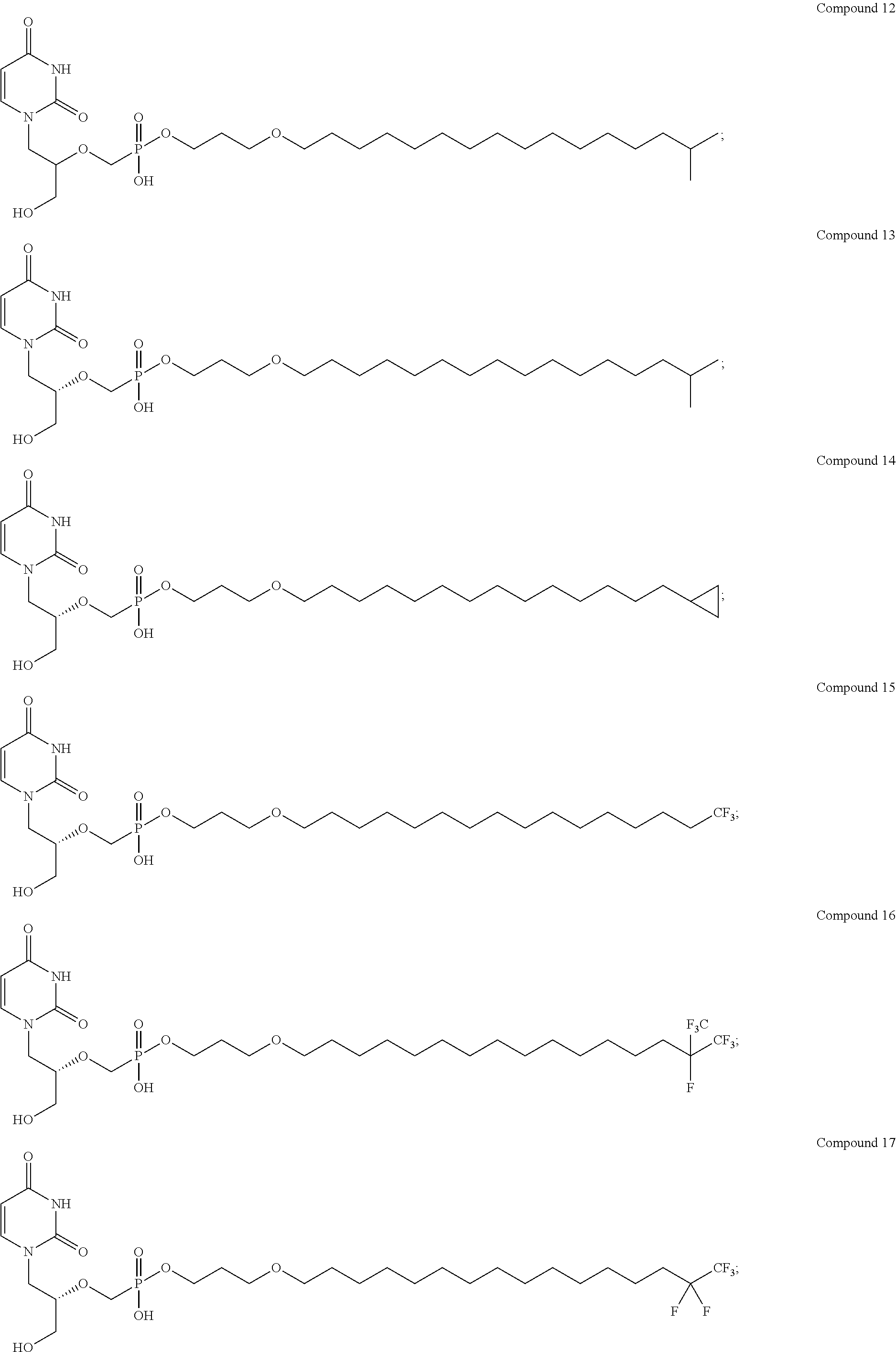 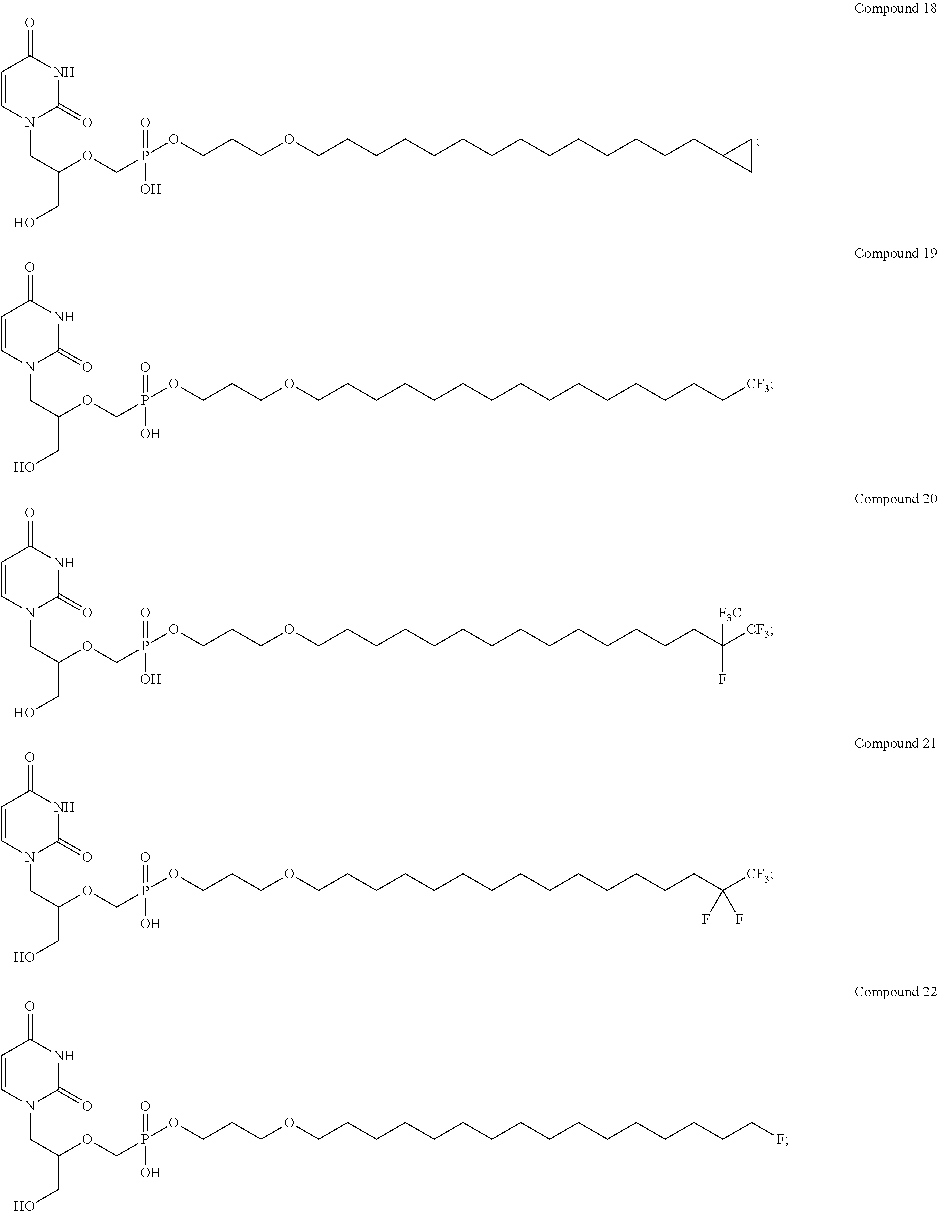 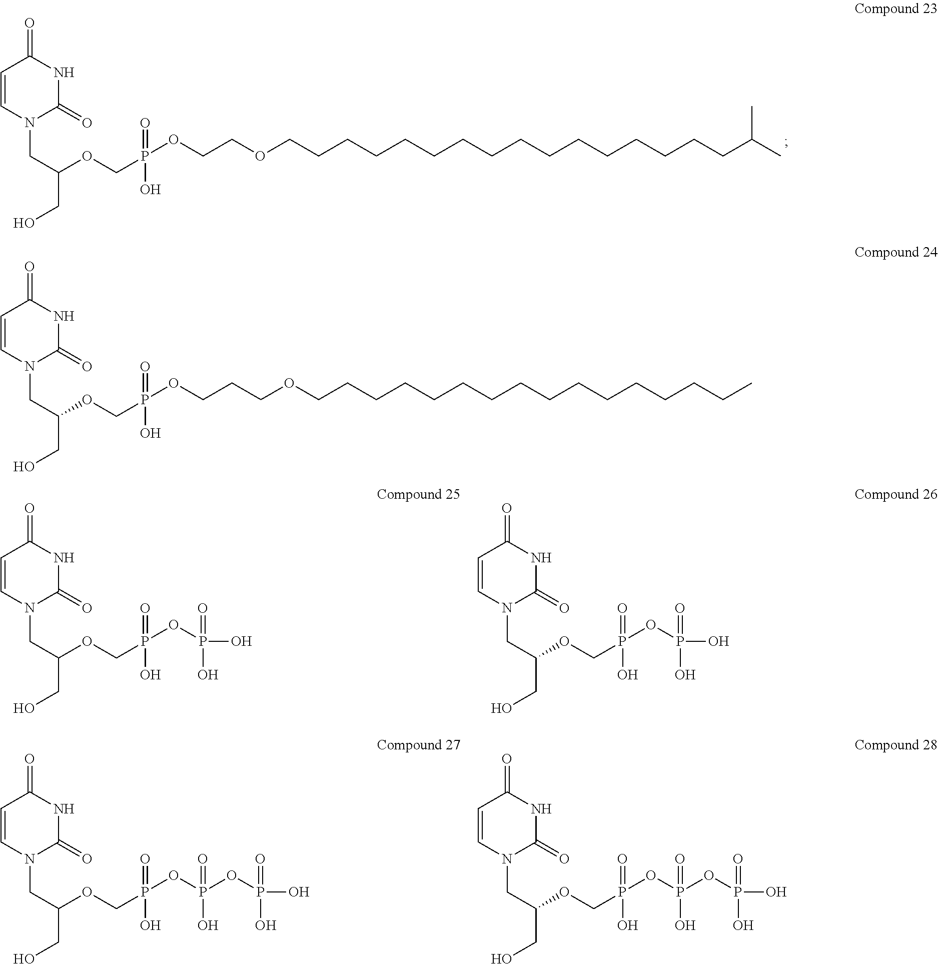
or a pharmaceutically acceptable salt thereof.

4. A method of synthesizing the compound of the formula: 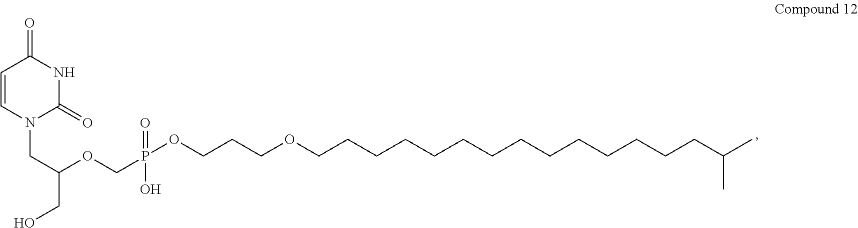 [SMILES: CC(C)CCCCCCCCCCCCCCOCCCOP(=O)(O)COC(CO)Cn1ccc(=O)[nH]c1=O]
wherein the method comprises the steps as follows:
(i) adding magnesium to a solution of 1-bromo-3-methyl butane in 2-methyltetrahydrofuran (Me-THF), followed by heating and then cooling the mixture;
(ii) adding 12-bromo-1-dodecanol in Me-THF to the reaction mixture of step (i), and adding immediately thereafter dilithium tetrachlorocuprate solution in tetrahydrofuran (THF);
(iii) back extracting the aqueous phase of the reaction mixture with ethyl acetate;
(iv) washing the organic solution with brine and drying over MgSO₄, filtering, and then concentrating in vacuo to produce Compound 3;
(v) adding mesyl chloride to a cold solution of Compound 3 and N,N-Diisopropylethylamine (DIPEA) in dichloromethane while maintaining the temperature below about 5° C.;
(vi) warming the reaction to room temperature and stirring for several hours;
(vii) adding mesyl chloride and DIPEA to the reaction mixture and stirring for several more hours;
(viii) adding water while cooling the reaction mixture, then separating the dichloromethane (DCM) layer from the aqueous layer, drying over a drying agent, and filtering the DCM layer to remove the drying agent;
(ix) concentrating the DCM solution in vacuo to give a yellow oil; and adding methanol to the concentrate of the yellow oil;
(x) filtering the precipitated solid and drying to yield Compound 4;
(xi) adding sodium hydride (NaH) to a cold solution of 1,3-propane diol in N-Methyl-2-pyrrolidone (NMP), and warming the mixture;
(xii) adding Compound 4 to the solution at step (xi) and stirring for several hours;
(xiii) adding water and ethyl acetate to the solution and separting the organic layer;
(xiv) concentrtaing the organic layer in vacuo, adding methanol and then drying;
(xv) adding acetonitrile, repeating step (xiv), forming Compound 5 after filtering and drying;
(xvi) adding trimethylsilyl bromide (TMS-Br) to a solution of Compound 6 in acetonitrile;
(xvii) adding dichloromethane after removing acetonitrile and TMS-Br and then concentrating;
(xviii) adding oxalyl chloride and Dimethylformamide (DMF) and concentrating in vacuo to form Compound 7;
(xix) mixing Compound 5 and Compound 7 in dichloromethane, and adding pyridine to the solution of Compound 7 and Compound 5;
(xx) separating the organic layer after adding water to the mixture in step (xix);
(xxi) separating the organic layer again after adding water and methanol to the organic layer in step (xx);
(xxii) drying the organic layer from step (xxi) in vacuo, and adding acetone;
(xxiii) drying and adding acetone before further drying and adjusting the pH to about 9.0;
(xxiv) adding acetone after filtering solid formed in step (xxiii);
(xxv) filtering and drying the solid product from step (xxiv) to produce Compound 8;
(xxvi) mixing Compound 8, Compound 9, magnesium di-tert butoxide in DMF;
(xxvii) adding isopropyl alcohol and hydrochloric acid (HCl) to the mixture before separating organic layer;
(xxviii) concentrating the organic layer and adding methanol to the mixture;
(xxix) concentrating the mixture of step (xxviii) to produce Compound 10;
(xxx) adding methanol to Compound 10 and filtering a triyl product;
(xxxi) diluting the filtrate in (xxx) with water, adjusting pH to about 2-3, and filtering to produce Compound 11;
(xxxii) adding water to Compound 11, and adjusting the pH to about 1-2 with HCl;
(xxxiii) extracting the aqueous solution of step (xxxii) with ethyl acetate;
(xxxiv) drying the ethyl acetate extract of step (xxxiv), filtering and concentrating to produce a thick oil; and
(xxxv) triturating the thick oil with acetone and cooling the solution, filtering, and drying to obtain Compound 12 as a white solid.

5. A pharmaceutical formulation of a compound of claim 1 or a pharmaceutically acceptable salt thereof comprising a carrier.

6.-8. (canceled)

9. A method of treating a viral infection and/or viral infection associated disease or disorder comprising administering a therapeutically effective amount of the compound of claim 1 or a pharmaceutically acceptable salt thereof to a subject in need thereof.

10. (canceled)

11. The method of claim 9, wherein the compound of claim 1 is administered in the amount of about 40-1000 mg.

12. (canceled)

13. The method of claim 9, wherein the compound of claim 1 is administered in the amount of about 40-400 mg.

14. The method of claim 9, wherein the compound of claim 1 is administered in the amount of about 100 mg.

15.-17. (canceled)

18. The method according to claim 9, wherein said subject is treated daily, once a week (QW) or twice a week (BIW) with about 40-1000 mg of said compound.

19.-31. (canceled)

32. The method of claim 9, wherein said compound of claim 1, or pharmaceutically acceptable salt thereof, is administered in combination with one or more compound or composition selected from the group consisting of an immunosuppressant and an antiviral agent.

33. The method of claim 32, wherein said compound of claim 1, or pharmaceutically acceptable salt thereof, is administered in combination with one or more compounds or compositions selected from the group consisting of: midazolam, cyclosporine A, tacrolimus, azoles, ganciclovir, valganciclovir, foscavir, cidofovir, second-line anti-HCV drugs, foscarnet, intravenously administered (IV) cidofovir, filgrastim, pegfilgrastim, corticosteroids such as budesonide, beclomethasone, and broad-spectrum CYP inhibitor aminobenzotriazole.

34. The method of claim 9, wherein the subject is immunocompromised.

35. The method of claim 34, wherein the immunocompromised subject is a transplant patient on immunosuppressive medications.

36. The method of claim 34, wherein the immunocompromised subject is infected with HIV.

37. The method of claim 34, wherein the compound is for administration in combination with at least one other immunosuppressant agent.

38. The method of claim 37, wherein the immunosuppressant agent is concurrently or sequentially administered.

39. The method of claim 37, wherein the immunosuppressant agent is selected from the group consisting of Daclizumab, Basiliximab, Tacrolimus, Sirolimus, Mycophenolate, Cyclosporine A, Glucocorticoids, Anti-CD3 monoclonal antibodies, Antithymocyte globulin, Anti-CD52 monoclonal antibodies, Azathioprine, Everolimus, Dactinomycin, Cyclophosphamide, Platinum, Nitrosurea, Methotrexate, Mercaptopurine, Muromonab, IFN gamma, Infliximab, Etanercept, Adalimumab, Natalizumab, Fingolimod, and combinations thereof.

40.-41. (canceled)

42. The method of claim 9, wherein the viral infection and/or viral infection associated disease or disorder is end-organ damage or impairment in a subject infected with BK virus, hepatitis C virus (HCV) infection, or a HCV infection associated disease or disorder.
